SEQUENCE LISTING (472 sequences)

SEQ ID NO: 1 - DNA, 20 bp, Human Papilloma Virus
catccgtaactacatcttcc

SEQ ID NO: 2 - DNA, 20 bp, Human Papilloma Virus
atccgtaactacatcttcca

SEQ ID NO: 3 - DNA, 22 bp, Human Papilloma Virus
ctacatcttccacatacaccaa

SEQ ID NO: 4 - DNA, 20 bp, Human Papilloma Virus
catcttccacatacaccaat

SEQ ID NO: 5 - DNA, 20 bp, Human Papilloma Virus
atcttccacatacaccaatt

SEQ ID NO: 6 - DNA, 20 bp, Human Papilloma Virus
ccacatacaccaattctgat

SEQ ID NO: 7 - DNA, 23 bp, Human Papilloma Virus
tagcattacattgtctgctgaag

SEQ ID NO: 8 - DNA, 19 bp, Human Papilloma Virus
tccctctgttttggaagac

SEQ ID NO: 9 - DNA, 26 bp, Human Papilloma Virus
gttatcgcctcccccaaatggtacat

SEQ ID NO: 10 - DNA, 22 bp, Human Papilloma Virus
ctataggtatgtgcagtcacag

SEQ ID NO: 11 - DNA, 19 bp, Human Papilloma Virus
gcccactcctgaaaaggaa

SEQ ID NO: 12 - DNA, 16 bp, Human Papilloma Virus
ctataagaaccttagt

SEQ ID NO: 13 - DNA, 15 bp, Human Papilloma Virus
atctgtgtctaaatc

SEQ ID NO: 14 - DNA, 20 bp, Human Papilloma Virus
tctgtgtctaaatctgctac

SEQ ID NO: 15 - DNA, 26 bp, Human Papilloma Virus
atctgtgtctaaatctgctacataca

SEQ ID NO: 16 - DNA, 20 bp, Human Papilloma Virus
tgcatctgtgtctaaatctg

SEQ ID NO: 17 - DNA, 20 bp, Human Papilloma Virus
aaatctgctacatacactaa

SEQ ID NO: 18 - DNA, 21 bp, Human Papilloma Virus
ctaaatctgctacatacacta

SEQ ID NO: 19 - DNA, 20 bp, Human Papilloma Virus
ctacatacactaattcagat

SEQ ID NO: 20 - DNA, 23 bp, Human Papilloma Virus
tagcattacattatctgcagaag

SEQ ID NO: 21 - DNA, 19 bp, Human Papilloma Virus
tccttctgttttggaggac

SEQ ID NO: 22 - DNA, 26 bp, Human Papilloma Virus
tttatcgcctccaccaaatggtacac

SEQ ID NO: 23 - DNA, 25 bp, Human Papilloma Virus
ttatagatatgtacagtcacaggcc

SEQ ID NO: 24 - DNA, 23 bp, Human Papilloma Virus
acccacacctgaaaaagaaaaac

SEQ ID NO: 25 - DNA, 20 bp, Human Papilloma Virus
tatgtcattatgtgctgcca

SEQ ID NO: 26 - DNA, 20 bp, Human Papilloma Virus
gtgctgccatatctacttca

SEQ ID NO: 27 - DNA, 15 bp, Human Papilloma Virus
tgccatatctacttc

SEQ ID NO: 28 - DNA, 20 bp, Human Papilloma Virus
tatctacttcagaaactaca

SEQ ID NO: 29 - DNA, 22 bp, Human Papilloma Virus
ctacttcagaaactacatataa

SEQ ID NO: 30 - DNA, 20 bp, Human Papilloma Virus
ataaaaatactaactttaag

SEQ ID NO: 31 - DNA, 23 bp, Human Papilloma Virus
caaaataaccttaactgcagacg

SEQ ID NO: 32 - DNA, 19 bp, Human Papilloma Virus
ttccactattttggaggac

SEQ ID NO: 33 - DNA, 26 bp, Human Papilloma Virus
tctacaacctcccccaggaggcacac

SEQ ID NO: 34 - DNA, 19 bp, Human Papilloma Virus
ttataggtttgtaacccag

SEQ ID NO: 35 - DNA, 19 bp, Human Papilloma Virus
acatacacctccagcacct

SEQ ID NO: 36 - DNA, 16 bp, Human Papilloma Virus
ccttaaaaaatacact

SEQ ID NO: 37 - DNA, 15 bp, Human Papilloma Virus
ttctacacagtctcc

SEQ ID NO: 38 - DNA, 20 bp, Human Papilloma Virus
cagtctcctgtacctgggca

SEQ ID NO: 39 - DNA, 20 bp, Human Papilloma Virus
agtctcctgtacctgggcaa

SEQ ID NO: 40 - DNA, 23 bp, Human Papilloma Virus
tctcctgtacctgggcaatatga

SEQ ID NO: 41 - DNA, 20 bp, Human Papilloma Virus
ctgtacctgggcaatatgat

SEQ ID NO: 42 - DNA, 20 bp, Human Papilloma Virus
atgatgctaccaaatttaag

SEQ ID NO: 43 - DNA, 23 bp, Human Papilloma Virus
tactattactttaactgcagatg

SEQ ID NO: 44 - DNA, 19 bp, Human Papilloma Virus
tagcagtattttagaggat

SEQ ID NO: 45 - DNA, 26 bp, Human Papilloma Virus
tgttcccccccccccaactactagtt

SEQ ID NO: 46 - DNA, 22 bp, Human Papilloma Virus
atatcgttttgtacaatctgtt

SEQ ID NO: 47 - DNA, 19 bp, Human Papilloma Virus
ggatgctgcaccggctgaa

SEQ ID NO: 48 - DNA, 16 bp, Human Papilloma Virus
ctatgataagttaaag

SEQ ID NO: 49 - DNA, 20 bp, Human Papilloma Virus
tagtacattatctgcagcat

SEQ ID NO: 50 - DNA, 15 bp, Human Papilloma Virus
attatctgcagcatc

SEQ ID NO: 51 - DNA, 20 bp, Human Papilloma Virus
tgcagcatctgcatccactc

SEQ ID NO: 52 - DNA, 24 bp, Human Papilloma Virus
gcatctgcatccactccatttaaa

SEQ ID NO: 53 - DNA, 20 bp, Human Papilloma Virus
ctccatttaaaccatctgat

SEQ ID NO: 54 - DNA, 23 bp, Human Papilloma Virus
taaaataacacttacaacagatg

SEQ ID NO: 55 - DNA, 19 bp, Human Papilloma Virus
tgcctccatattggaggat

SEQ ID NO: 56 - DNA, 26 bp, Human Papilloma Virus
actaaccttacctcccactgctagtt

SEQ ID NO: 57 - DNA, 22 bp, Human Papilloma Virus
ctataggtttattaaaaactct

SEQ ID NO: 58 - DNA, 19 bp, Human Papilloma Virus
taacgcccctcctgtgcca

SEQ ID NO: 59 - DNA, 15 bp, Human Papilloma Virus
tgcaattgcaaacag

SEQ ID NO: 60 - DNA, 20 bp, Human Papilloma Virus
gcaattgcaaacagtgatac

SEQ ID NO: 61 - DNA, 20 bp, Human Papilloma Virus
caattgcaaacagtgatact

SEQ ID NO: 62 - DNA, 23 bp, Human Papilloma Virus
gcaaacagtgatactacatttaa

SEQ ID NO: 63 - DNA, 20 bp, Human Papilloma Virus
ctacatttaaaagtagtaat

SEQ ID NO: 64 - DNA, 23 bp, Human Papilloma Virus
caaaataacattatctgcagaca

SEQ ID NO: 65 - DNA, 19 bp, Human Papilloma Virus
tcctgctattttggaagat

SEQ ID NO: 66 - DNA, 26 bp, Human Papilloma Virus
attgaccacacctccctcaggttctt

SEQ ID NO: 67 - DNA, 22 bp, Human Papilloma Virus
ctataggtttgtcacctcacag

SEQ ID NO: 68 - DNA, 19 bp, Human Papilloma Virus
aactgccccccaaaagccc

SEQ ID NO: 69 - DNA, 20 bp, Human Papilloma Virus
tgctactgtaacaactgaag

SEQ ID NO: 70 - DNA, 20 bp, Human Papilloma Virus
gctactgtaacaactgaaga

SEQ ID NO: 71 - DNA, 15 bp, Human Papilloma Virus
tactgtaacaactga

SEQ ID NO: 72 - DNA, 20 bp, Human Papilloma Virus
actgtaacaactgaagacac

SEQ ID NO: 73 - DNA, 22 bp, Human Papilloma Virus
caactgaagacacatacaagtc

SEQ ID NO: 74 - DNA, 23 bp, Human Papilloma Virus
caaaattacattatctgtagagg

SEQ ID NO: 75 - DNA, 19 bp, Human Papilloma Virus
tcctgacatactagacgat

SEQ ID NO: 76 - DNA, 26 bp, Human Papilloma Virus
tgtagctccaccgccctctggtactt

SEQ ID NO: 77 - DNA, 22 bp, Human Papilloma Virus
ttatagatttgtgcagtctcag

SEQ ID NO: 78 - DNA, 19 bp, Human Papilloma Virus
taaggtaacagcacctgaa

SEQ ID NO: 79 - DNA, 16 bp, Human Papilloma Virus
tttttctgactattca

SEQ ID NO: 80 - DNA, 20 bp, Human Papilloma Virus
tatgcacacaagtaactagt

SEQ ID NO: 81 - DNA, 15 bp, Human Papilloma Virus
cacacaagtaactag

SEQ ID NO: 82 - DNA, 20 bp, Human Papilloma Virus
acaagtaactagtgacagta

SEQ ID NO: 83 - DNA, 23 bp, Human Papilloma Virus
gtaactagtgacagtacatataa

SEQ ID NO: 84 - DNA, 20 bp, Human Papilloma Virus
gtacatataaaaatgaaaat

SEQ ID NO: 85 - DNA, 23 bp, Human Papilloma Virus
caaagttaccttaactgcagaag

SEQ ID NO: 86 - DNA, 19 bp, Human Papilloma Virus
tccagatattttagaagat

SEQ ID NO: 87 - DNA, 26 bp, Human Papilloma Virus
tttaacacctcctccatctgctagtt

SEQ ID NO: 88 - DNA, 22 bp, Human Papilloma Virus
ctataggtttgttacctctcag

SEQ ID NO: 89 - DNA, 19 bp, Human Papilloma Virus
aacagtacctccaaaggaa

SEQ ID NO: 90 - DNA, 16 bp, Human Papilloma Virus
cttaggtaaatataca

SEQ ID NO: 91 - DNA, 20 bp, Human Papilloma Virus
tctgctgtgtcttctagtga

SEQ ID NO: 92 - DNA, 15 bp, Human Papilloma Virus
tgctgtgtcttctag

SEQ ID NO: 93 - DNA, 20 bp, Human Papilloma Virus
gtgtcttctagtgacagtac

SEQ ID NO: 94 - DNA, 23 bp, Human Papilloma Virus
cttctagtgacagtacatataaa

SEQ ID NO: 95 - DNA, 20 bp, Human Papilloma Virus
gtacatataaaaatgacaat

SEQ ID NO: 96 - DNA, 23 bp, Human Papilloma Virus
taaaataacactaacagcagatg

SEQ ID NO: 97 - DNA, 19 bp, Human Papilloma Virus
cccgtccattttagaggat

SEQ ID NO: 98 - DNA, 26 bp, Human Papilloma Virus
ccttacaccaccgccttctggtacct

SEQ ID NO: 99 - DNA, 22 bp, Human Papilloma Virus
atatcgctatgtaacatcacag

SEQ ID NO: 100 - DNA, 19 bp, Human Papilloma Virus
acccagtgcaccaaaacct

SEQ ID NO: 101 - DNA, 15 bp, Human Papilloma Virus
tgtctactgacaatg

SEQ ID NO: 102 - DNA, 20 bp, Human Papilloma Virus
tgtctactgacaatggcgaa

SEQ ID NO: 103 - DNA, 20 bp, Human Papilloma Virus
tgacaatggcgaagttacag

SEQ ID NO: 104 - DNA, 20 bp, Human Papilloma Virus
gacaatggcgaagttacaga

SEQ ID NO: 105 - DNA, 20 bp, Human Papilloma Virus
aatggcgaagttacagaata

SEQ ID NO: 106 - DNA, 20 bp, Human Papilloma Virus
cagaatataattctcaaaca

SEQ ID NO: 107 - DNA, 23 bp, Human Papilloma Virus
taaagttcctttaaaggctgagg

SEQ ID NO: 108 - DNA, 19 bp, Human Papilloma Virus
ttctggtatattggaagag

SEQ ID NO: 109 - DNA, 26 bp, Human Papilloma Virus
atttgtacctactccagataattcag

SEQ ID NO: 110 - DNA, 22 bp, Human Papilloma Virus
ttataggtacattaattcaaag

SEQ ID NO: 111 - DNA, 19 bp, Human Papilloma Virus
tgcagttgttgaaaaagaa

SEQ ID NO: 112 - DNA, 16 bp, Human Papilloma Virus
ctttgcaaaatataca

SEQ ID NO: 113 - DNA, 15 bp, Human Papilloma Virus
ctctatagagtcttc

SEQ ID NO: 114 - DNA, 20 bp, Human Papilloma Virus
tagagtcttccataccttct

SEQ ID NO: 115 - DNA, 20 bp, Human Papilloma Virus
atagagtcttccataccttc

SEQ ID NO: 116 - DNA, 23 bp, Human Papilloma Virus
gtcttccataccttctacatatg

SEQ ID NO: 117 - DNA, 20 bp, Human Papilloma Virus
ctacatatgatccttctaag

SEQ ID NO: 118 - DNA, 23 bp, Human Papilloma Virus
tactgtcacattaacaactgatg

SEQ ID NO: 119 - DNA, 18 bp, Human Papilloma Virus
ttcctctatattggacaa

SEQ ID NO: 120 - DNA, 26 bp, Human Papilloma Virus
tgtagctcctccaccatctgccagtt

SEQ ID NO: 121 - DNA, 22 bp, Human Papilloma Virus
ttacagatacctacagtctgca

SEQ ID NO: 122 - DNA, 19 bp, Human Papilloma Virus
ggatgctccagcacctgaa

SEQ ID NO: 123 - DNA, 16 bp, Human Papilloma Virus
atatgacggtctaaag

SEQ ID NO: 124 - DNA, 22 bp, Human Papilloma Virus
tatatgttggggaaatcagcta

SEQ ID NO: 125 - DNA, 20 bp, Human Papilloma Virus
cactgcaacatctggtgata

SEQ ID NO: 126 - DNA, 30 bp, Human Papilloma Virus
gcaacatctggtgatacatatacagctgct

SEQ ID NO: 127 - DNA, 23 bp, Human Papilloma Virus
cattaactgttgaagttatgtca

SEQ ID NO: 128 - DNA, 26 bp, Human Papilloma Virus
cctaacatattagaggagtggaatgt

SEQ ID NO: 129 - DNA, 20 bp, Human Papilloma Virus
caccaccaccttcaggaact

SEQ ID NO: 130 - DNA, 24 bp, Human Papilloma Virus
gttataggtatgtacaatcagaag

SEQ ID NO: 131 - DNA, 30 bp, Human Papilloma Virus
gctaaggtaacaacgccagaaaaaaaggat

SEQ ID NO: 132 - DNA, 24 bp, Human Papilloma Virus
cagacttttggttttgggaggtaa

SEQ ID NO: 133 - DNA, 21 bp, Human Papilloma Virus
gaaaagttttctactgattta

SEQ ID NO: 134 - DNA, 22 bp, Human Papilloma Virus
catttgttttgggaatcagttg

SEQ ID NO: 135 - DNA, 20 bp, Human Papilloma Virus
tgaccctactgtgcccagta

SEQ ID NO: 136 - DNA, 30 bp, Human Papilloma Virus
actgtgcccagtacatatgacaatgcaaag

SEQ ID NO: 137 - DNA, 23 bp, Human Papilloma Virus
gtttatatttcaattatgcataa

SEQ ID NO: 138 - DNA, 21 bp, Human Papilloma Virus
ccagaggttatgacatatatt

SEQ ID NO: 139 - DNA, 23 bp, Human Papilloma Virus
cccacattattagaggactggaa

SEQ ID NO: 140 - DNA, 21 bp, Human Papilloma Virus
ccacctgcctctgcttctttg

SEQ ID NO: 141 - DNA, 25 bp, Human Papilloma Virus
cgctttttgtctaacaaggccattg

SEQ ID NO: 142 - DNA, 23 bp, Human Papilloma Virus
ccaaaggaacgggaggatcccta

SEQ ID NO: 143 - DNA, 25 bp, Human Papilloma Virus
cttacagaaaagttttctgcacaac

SEQ ID NO: 144 - DNA, 15 bp, Human Papilloma Virus
tgccactacacagtc

SEQ ID NO: 145 - DNA, 20 bp, Human Papilloma Virus
ctacacagtcccctccgtct

SEQ ID NO: 146 - DNA, 20 bp, Human Papilloma Virus
tgccactacacagtcccctc

SEQ ID NO: 147 - DNA, 22 bp, Human Papilloma Virus
cagtcccctccgtctacatata

SEQ ID NO: 148 - DNA, 20 bp, Human Papilloma Virus
ctacatatactagtgaacaa

SEQ ID NO: 149 - DNA, 23 bp, Human Papilloma Virus
tagtattaccttaacggcggagg

SEQ ID NO: 150 - DNA, 19 bp, Human Papilloma Virus
tgctggtattttagaacag

SEQ ID NO: 151 - DNA, 26 bp, Human Papilloma Virus
gttgtcgccgcccccaaatggtacct

SEQ ID NO: 152 - DNA, 22 bp, Human Papilloma Virus
atacagatatgtgcagtcccag

SEQ ID NO: 153 - DNA, 19 bp, Human Papilloma Virus
gccaccccctgaaaaggca

SEQ ID NO: 154 - DNA, 16 bp, Human Papilloma Virus
ctatgcaaaattaagt

SEQ ID NO: 155 - DNA, 20 bp, Human Papilloma Virus
tgcctctacacaaaatcctg

SEQ ID NO: 156 - DNA, 15 bp, Human Papilloma Virus
ctctacacaaaatcc

SEQ ID NO: 157 - DNA, 20 bp, Human Papilloma Virus
acaaaatcctgtgccaagta

SEQ ID NO: 158 - DNA, 20 bp, Human Papilloma Virus
caaaatcctgtgccaagtac

SEQ ID NO: 159 - DNA, 22 bp, Human Papilloma Virus
aatcctgtgccaagtacatatg

SEQ ID NO: 160 - DNA, 20 bp, Human Papilloma Virus
gtacatatgaccctactaag

SEQ ID NO: 161 - DNA, 23 bp, Human Papilloma Virus
cactattactttaactgcagagg

SEQ ID NO: 162 - DNA, 19 bp, Human Papilloma Virus
tagtagtatattagaaaat

SEQ ID NO: 163 - DNA, 26 bp, Human Papilloma Virus
tgtccctccaccacctactacaagtt

SEQ ID NO: 164 - DNA, 22 bp, Human Papilloma Virus
atatcgttttgtgcaatcagtt

SEQ ID NO: 165 - DNA, 19 bp, Human Papilloma Virus
ggatactacacctccagaa

SEQ ID NO: 166 - DNA, 20 bp, Human Papilloma Virus
cactgccactgctgcggttt

SEQ ID NO: 167 - DNA, 15 bp, Human Papilloma Virus
tgccactgctgcggt

SEQ ID NO: 168 - DNA, 20 bp, Human Papilloma Virus
cactgctgcggtttccccaa

SEQ ID NO: 169 - DNA, 20 bp, Human Papilloma Virus
ccactgctgcggtttcccca

SEQ ID NO: 170 - DNA, 22 bp, Human Papilloma Virus
ctgcggtttccccaacatttac

SEQ ID NO: 171 - DNA, 20 bp, Human Papilloma Virus
caacatttactccaagtaac

SEQ ID NO: 172 - DNA, 23 bp, Human Papilloma Virus
taaaattactttaactacagagg

SEQ ID NO: 173 - DNA, 19 bp, Human Papilloma Virus
tcctaccattcttgaacag

SEQ ID NO: 174 - DNA, 26 bp, Human Papilloma Virus
attaacattacctccgtctgctagtt

SEQ ID NO: 175 - DNA, 22 bp, Human Papilloma Virus
atataggtttgttagaaatgca

SEQ ID NO: 176 - DNA, 19 bp, Human Papilloma Virus
ggacacccctccacaggct

SEQ ID NO: 177 - DNA, 16 bp, Human Papilloma Virus
tttggccaaatataaa

SEQ ID NO: 178 - DNA, 15 bp, Human Papilloma Virus
tgaggttaaaaagga

SEQ ID NO: 179 - DNA, 20 bp, Human Papilloma Virus
tgaggttaaaaaggaaagca

SEQ ID NO: 180 - DNA, 20 bp, Human Papilloma Virus
gaggttaaaaaggaaagcac

SEQ ID NO: 181 - DNA, 20 bp, Human Papilloma Virus
ttaaaaaggaaagcacatat

SEQ ID NO: 182 - DNA, 22 bp, Human Papilloma Virus
aaaggaaagcacatataaaaat

SEQ ID NO: 183 - DNA, 20 bp, Human Papilloma Virus
gcacatataaaaatgaaaat

SEQ ID NO: 184 - DNA, 23 bp, Human Papilloma Virus
caaaattacattaacagctgatg

SEQ ID NO: 185 - DNA, 19 bp, Human Papilloma Virus
tgccactattttagaggac

SEQ ID NO: 186 - DNA, 26 bp, Human Papilloma Virus
ccttaccccaccaccgtctgcatctt

SEQ ID NO: 187 - DNA, 22 bp, Human Papilloma Virus
atacagatttgtcacttctact

SEQ ID NO: 188 - DNA, 19 bp, Human Papilloma Virus
aaacacaccacctaaagga

SEQ ID NO: 189 - DNA, 16 bp, Human Papilloma Virus
tttaaaggactatatg

SEQ ID NO: 190 - DNA, 20 bp, Human Papilloma Virus
tccgcaaccacacagtctat

SEQ ID NO: 191 - DNA, 15 bp, Human Papilloma Virus
ccgcaaccacacagt

SEQ ID NO: 192 - DNA, 20 bp, Human Papilloma Virus
ccgcaaccacacagtctatg

SEQ ID NO: 193 - DNA, 22 bp, Human Papilloma Virus
cacagtctatgtctacatataa

SEQ ID NO: 194 - DNA, 20 bp, Human Papilloma Virus
ctacatataattcaaagcaa

SEQ ID NO: 195 - DNA, 23 bp, Human Papilloma Virus
taaaatatccctgtctgctgagg

SEQ ID NO: 196 - DNA, 19 bp, Human Papilloma Virus
ttctaccttactggaagac

SEQ ID NO: 197 - DNA, 26 bp, Human Papilloma Virus
tttgtcgcctcctgttgccactagct

SEQ ID NO: 198 - DNA, 22 bp, Human Papilloma Virus
atacagatatgtgaaaagtgca

SEQ ID NO: 199 - DNA, 19 bp, Human Papilloma Virus
ggatcagccccctcctgaa

SEQ ID NO: 200 - DNA, 15 bp, Human Papilloma Virus
tacagcatccacgca

SEQ ID NO: 201 - DNA, 20 bp, Human Papilloma Virus
cagcatccacgcaggatagc

SEQ ID NO: 202 - DNA, 20 bp, Human Papilloma Virus
acgcaggatagctttaataa

SEQ ID NO: 203 - DNA, 20 bp, Human Papilloma Virus
cacgcaggatagctttaata

SEQ ID NO: 204 - DNA, 20 bp, Human Papilloma Virus
atagctttaataattctgac

SEQ ID NO: 205 - DNA, 23 bp, Human Papilloma Virus
taccataacccttacagcagatg

SEQ ID NO: 206 - DNA, 19 bp, Human Papilloma Virus
tcccactattctagaggac

SEQ ID NO: 207 - DNA, 26 bp, Human Papilloma Virus
tataacccccccagctacaagtagtt

SEQ ID NO: 208 - DNA, 22 bp, Human Papilloma Virus
atataggtttgtacagtcacag

SEQ ID NO: 209 - DNA, 22 bp, Human Papilloma Virus
gaataatgcccctgcaaaggaa

SEQ ID NO: 210 - DNA, 23 bp, Human Papilloma Virus
tttgttactgttgtagatactac

SEQ ID NO: 211 - DNA, 20 bp, Human Papilloma Virus
atgacaatatgtgctgctac

SEQ ID NO: 212 - DNA, 20 bp, Human Papilloma Virus
gacaatatgtgctgctacaa

SEQ ID NO: 213 - DNA, 20 bp, Human Papilloma Virus
tgctacaactcagtctccat

SEQ ID NO: 214 - DNA, 20 bp, Human Papilloma Virus
ctacaactcagtctccatct

SEQ ID NO: 215 - DNA, 20 bp, Human Papilloma Virus
acaactcagtctccatctac

SEQ ID NO: 216 - DNA, 20 bp, Human Papilloma Virus
atgttgaggagtttgactta

SEQ ID NO: 217 - DNA, 20 bp, Human Papilloma Virus
tgttgaggagtttgacttac

SEQ ID NO: 218 - DNA, 20 bp, Human Papilloma Virus
tgaggagtttgacttacagt

SEQ ID NO: 219 - DNA, 15 bp, Human Papilloma Virus
ctgctacagaacagt

SEQ ID NO: 220 - DNA, 20 bp, Human Papilloma Virus
gctacagaacagttaagtaa

SEQ ID NO: 221 - DNA, 20 bp, Human Papilloma Virus
cagaacagttaagtaaatat

SEQ ID NO: 222 - DNA, 23 bp, Human Papilloma Virus
gaacagttaagtaaatatgatgc

SEQ ID NO: 223 - DNA, 20 bp, Human Papilloma Virus
gtaaatatgatgcacgaaaa

SEQ ID NO: 224 - DNA, 23 bp, Human Papilloma Virus
caaaattactttgtctgcagagg

SEQ ID NO: 225 - DNA, 19 bp, Human Papilloma Virus
tgctaacctactggaggac

SEQ ID NO: 226 - DNA, 26 bp, Human Papilloma Virus
gttatccccgccagtggccaccagcc

SEQ ID NO: 227 - DNA, 22 bp, Human Papilloma Virus
atatagatatgttagaagcaca

SEQ ID NO: 228 - DNA, 19 bp, Human Papilloma Virus
ggaacagccaccaacagaa

SEQ ID NO: 229 - DNA, 20 bp, Human Papilloma Virus
atgcactgaagtaactaagg

SEQ ID NO: 230 - DNA, 20 bp, Human Papilloma Virus
cactgaagtaactaaggaag

SEQ ID NO: 231 - DNA, 15 bp, Human Papilloma Virus
tgaagtaactaagga

SEQ ID NO: 232 - DNA, 20 bp, Human Papilloma Virus
gaagtaactaaggaaggtac

SEQ ID NO: 233 - DNA, 22 bp, Human Papilloma Virus
ctaaggaaggtacatataaaaa

SEQ ID NO: 234 - DNA, 20 bp, Human Papilloma Virus
ataaaaatgataattttaag

SEQ ID NO: 235 - DNA, 23 bp, Human Papilloma Virus
caaaattacactaactgcagaga

SEQ ID NO: 236 - DNA, 19 bp, Human Papilloma Virus
ttccaatattttggaggac

SEQ ID NO: 237 - DNA, 26 bp, Human Papilloma Virus
tttaacacctcctccgtctgccagtt

SEQ ID NO: 238 - DNA, 22 bp, Human Papilloma Virus
atatagatttgttacctcccag

SEQ ID NO: 239 - DNA, 19 bp, Human Papilloma Virus
aacagcaccccctaaagaa

SEQ ID NO: 240 - DNA, 15 bp, Human Papilloma Virus
ttctactacttcttc

SEQ ID NO: 241 - DNA, 20 bp, Human Papilloma Virus
actacttcttctattcctaa

SEQ ID NO: 242 - DNA, 20 bp, Human Papilloma Virus
acttcttctattcctaatgt

SEQ ID NO: 243 - DNA, 23 bp, Human Papilloma Virus
tcttctattcctaatgtatacac

SEQ ID NO: 244 - DNA, 20 bp, Human Papilloma Virus
atgtatacacacctaccagt

SEQ ID NO: 245 - DNA, 23 bp, Human Papilloma Virus
taaaataacattaactacagagg

SEQ ID NO: 246 - DNA, 19 bp, Human Papilloma Virus
taccactattttggaggat

SEQ ID NO: 247 - DNA, 26 bp, Human Papilloma Virus
tgttacaccacctcctactgctagtt

SEQ ID NO: 248 - DNA, 22 bp, Human Papilloma Virus
ataccgttttgttcaatctgct

SEQ ID NO: 249 - DNA, 19 bp, Human Papilloma Virus
ggacaccgcaccgccagtt

SEQ ID NO: 250 - DNA, 16 bp, Human Papilloma Virus
ttatgacaaactaaag

SEQ ID NO: 251 - DNA, 15 bp, Human Papilloma Virus
ctgctacatcccccc

SEQ ID NO: 252 - DNA, 20 bp, Human Papilloma Virus
acatccccccctgtatctga

SEQ ID NO: 253 - DNA, 20 bp, Human Papilloma Virus
catccccccctgtatctgaa

SEQ ID NO: 254 - DNA, 22 bp, Human Papilloma Virus
cccctgtatctgaatataaagc

SEQ ID NO: 255 - DNA, 20 bp, Human Papilloma Virus
ctgaatataaagccacaagc

SEQ ID NO: 256 - DNA, 23 bp, Human Papilloma Virus
taaaatacatttaacccctgaaa

SEQ ID NO: 257 - DNA, 19 bp, Human Papilloma Virus
taaggccttgttggatgac

SEQ ID NO: 258 - DNA, 26 bp, Human Papilloma Virus
tgtggtaccaccaccctctaccagtt

SEQ ID NO: 259 - DNA, 22 bp, Human Papilloma Virus
atataggtttttgcagtccaga

SEQ ID NO: 260 - DNA, 22 bp, Human Papilloma Virus
gggtgctgctgccccgccgccc

SEQ ID NO: 261 - DNA, 16 bp, Human Papilloma Virus
ctatgccaagttatcc

SEQ ID NO: 262 - DNA, 15 bp, Human Papilloma Virus
ccgcctccactgctg

SEQ ID NO: 263 - DNA, 20 bp, Human Papilloma Virus
gcctccactgctgcagcaga

SEQ ID NO: 264 - DNA, 20 bp, Human Papilloma Virus
ctgctgcagcagaatacacg

SEQ ID NO: 265 - DNA, 20 bp, Human Papilloma Virus
gcagaatacacggctaccaa

SEQ ID NO: 266 - DNA, 20 bp, Human Papilloma Virus
cagaatacacggctaccaac

SEQ ID NO: 267 - DNA, 23 bp, Human Papilloma Virus
caaaatacagttaacccccgaaa

SEQ ID NO: 268 - DNA, 19 bp, Human Papilloma Virus
caaggaccttttggatgac

SEQ ID NO: 269 - DNA, 26 bp, Human Papilloma Virus
ggttttacctcccccttccactagtt

SEQ ID NO: 270 - DNA, 22 bp, Human Papilloma Virus
atatcactatttcgagtctcgg

SEQ ID NO: 271 - DNA, 19 bp, Human Papilloma Virus
ggggctgcctacccgtccc

SEQ ID NO: 272 - DNA, 16 bp, Human Papilloma Virus
gtatgcgcaaatgaca

SEQ ID NO: 273 - DNA, 15 bp, Human Papilloma Virus
cagctaaaagcacat

SEQ ID NO: 274 - DNA, 20 bp, Human Papilloma Virus
cagctaaaagcacattaact

SEQ ID NO: 275 - DNA, 20 bp, Human Papilloma Virus
ctaaaagcacattaactaaa

SEQ ID NO: 276 - DNA, 20 bp, Human Papilloma Virus
ttaactaaatatgatgcccg

SEQ ID NO: 277 - DNA, 20 bp, Human Papilloma Virus
ctaaatatgatgcccgtgaa

SEQ ID NO: 278 - DNA, 23 bp, Human Papilloma Virus
taaaataaccttaactgcagaag

SEQ ID NO: 279 - DNA, 19 bp, Human Papilloma Virus
taatactttattagacgat

SEQ ID NO: 280 - DNA, 26 bp, Human Papilloma Virus
cttatccccaccagttgcaactagct

SEQ ID NO: 281 - DNA, 22 bp, Human Papilloma Virus
atataggtatattaaaagcaca

SEQ ID NO: 282 - DNA, 19 bp, Human Papilloma Virus
ggaacagccccctgcagaa

SEQ ID NO: 283 - DNA, 16 bp, Human Papilloma Virus
cctggctaaatataag

SEQ ID NO: 284 - DNA, 15 bp, Human Papilloma Virus
ctgaggaaaaatcag

SEQ ID NO: 285 - DNA, 20 bp, Human Papilloma Virus
gaggaaaaatcagaggctac

SEQ ID NO: 286 - DNA, 23 bp, Human Papilloma Virus
atcagaggctacatacaaaaatg

SEQ ID NO: 287 - DNA, 20 bp, Human Papilloma Virus
aggaaaaatcagaggctaca

SEQ ID NO: 288 - DNA, 20 bp, Human Papilloma Virus
ctacatacaaaaatgaaaac

SEQ ID NO: 289 - DNA, 23 bp, Human Papilloma Virus
caaaatatcccttactgcaaatg

SEQ ID NO: 290 - DNA, 19 bp, Human Papilloma Virus
tccagatatattagaggac

SEQ ID NO: 291 - DNA, 26 bp, Human Papilloma Virus
ccttacaccacctccttcaggtaatt

SEQ ID NO: 292 - DNA, 22 bp, Human Papilloma Virus
atatagatttgttacctcgcag

SEQ ID NO: 293 - DNA, 19 bp, Human Papilloma Virus
aacatcccctccaacagca

SEQ ID NO: 294 - DNA, 16 bp, Human Papilloma Virus
tcttaaaaagtacagt

SEQ ID NO: 295 - DNA, 15 bp, Human Papilloma Virus
ctactactgaatcag

SEQ ID NO: 296 - DNA, 20 bp, Human Papilloma Virus
tgaatcagctgtaccaaata

SEQ ID NO: 297 - DNA, 20 bp, Human Papilloma Virus
gaatcagctgtaccaaatat

SEQ ID NO: 298 - DNA, 22 bp, Human Papilloma Virus
cagctgtaccaaatatttatga

SEQ ID NO: 299 - DNA, 20 bp, Human Papilloma Virus
atatttatgatcctaataaa

SEQ ID NO: 300 - DNA, 19 bp, Human Papilloma Virus
tcctgctattttggatgat

SEQ ID NO: 301 - DNA, 23 bp, Human Papilloma Virus
tactataacattgtccactgatg

SEQ ID NO: 302 - DNA, 26 bp, Human Papilloma Virus
tgttgcccctccaccatctgctagtc

SEQ ID NO: 303 - DNA, 22 bp, Human Papilloma Virus
ataccgctatctgcaatcagca

SEQ ID NO: 304 - DNA, 19 bp, Human Papilloma Virus
agacgcccctgcacctact

SEQ ID NO: 305 - DNA, 16 bp, Human Papilloma Virus
atatgatggcttaaac

SEQ ID NO: 306 - DNA, 20 bp, Human Papilloma Virus
tattagtactgtatctgcac

SEQ ID NO: 307 - DNA, 15 bp, Human Papilloma Virus
ctgtatctgcacaat

SEQ ID NO: 308 - DNA, 20 bp, Human Papilloma Virus
ctgtatctgcacaatctgca

SEQ ID NO: 309 - DNA, 20 bp, Human Papilloma Virus
tgcacaatctgcatctgcca

SEQ ID NO: 310 - DNA, 22 bp, Human Papilloma Virus
caatctgcatctgccactttta

SEQ ID NO: 311 - DNA, 20 bp, Human Papilloma Virus
ccacttttaaaccatcagat

SEQ ID NO: 312 - DNA, 23 bp, Human Papilloma Virus
taaaattactcttaccactgatg

SEQ ID NO: 313 - DNA, 19 bp, Human Papilloma Virus
ttctactattttggaaaat

SEQ ID NO: 314 - DNA, 26 bp, Human Papilloma Virus
ccttaccttgcctcctactgctagtt

SEQ ID NO: 315 - DNA, 22 bp, Human Papilloma Virus
atataggtttattaaaaattca

SEQ ID NO: 316 - DNA, 19 bp, Human Papilloma Virus
cgatgcccctgcacagccc

SEQ ID NO: 317 - DNA, 20 bp, Human Papilloma Virus
tgtctgcctgcaccgaaacg

SEQ ID NO: 318 - DNA, 15 bp, Human Papilloma Virus
ctgcaccgaaacggc

SEQ ID NO: 319 - DNA, 20 bp, Human Papilloma Virus
gaaacggccatacctgctgt

SEQ ID NO: 320 - DNA, 20 bp, Human Papilloma Virus
cgaaacggccatacctgctg

SEQ ID NO: 321 - DNA, 22 bp, Human Papilloma Virus
cggccatacctgctgtatatag

SEQ ID NO: 322 - DNA, 20 bp, Human Papilloma Virus
ctgtatatagccctacaaag

SEQ ID NO: 323 - DNA, 23 bp, Human Papilloma Virus
tactatcacattaactgctgacg

SEQ ID NO: 324 - DNA, 19 bp, Human Papilloma Virus
tcctgcaattttggacaat

SEQ ID NO: 325 - DNA, 26 bp, Human Papilloma Virus
agttacccctccaccatctgcaagct

SEQ ID NO: 326 - DNA, 22 bp, Human Papilloma Virus
gtataggtatttacaatcagca

SEQ ID NO: 327 - DNA, 19 bp, Human Papilloma Virus
ggatgctcctacacctgaa

SEQ ID NO: 328 - DNA, 16 bp, Human Papilloma Virus
ctatgacgatttaaaa

SEQ ID NO: 329 - DNA, 20 bp, Human Papilloma Virus
atctgttggtttaatgagct

SEQ ID NO: 330 - DNA, 20 bp, Human Papilloma Virus
tttgtgacagttgtagatac

SEQ ID NO: 331 - DNA, 15 bp, Human Papilloma Virus
ctgccacagcgtcct

SEQ ID NO: 332 - DNA, 20 bp, Human Papilloma Virus
acagcgtcctctgtatcaga

SEQ ID NO: 333 - DNA, 20 bp, Human Papilloma Virus
ccacagcgtcctctgtatca

SEQ ID NO: 334 - DNA, 22 bp, Human Papilloma Virus
agcgtcctctgtatcagaatat

SEQ ID NO: 335 - DNA, 20 bp, Human Papilloma Virus
cagaatatacagcttctaat

SEQ ID NO: 336 - DNA, 23 bp, Human Papilloma Virus
taaaattcacttaactcctgaaa

SEQ ID NO: 337 - DNA, 19 bp, Human Papilloma Virus
taaggccttattggatgac

SEQ ID NO: 338 - DNA, 26 bp, Human Papilloma Virus
tgtggtgcctcctccttctaccagtt

SEQ ID NO: 339 - DNA, 22 bp, Human Papilloma Virus
ctataggtttttgcagtctcgt

SEQ ID NO: 340 - DNA, 22 bp, Human Papilloma Virus
gggggctgccacccctcctcct

SEQ ID NO: 341 - DNA, 16 bp, Human Papilloma Virus
atatgctaacttatcc

SEQ ID NO: 342 - DNA, 15 bp, Human Papilloma Virus
cctacctcacaatcg

SEQ ID NO: 343 - DNA, 20 bp, Human Papilloma Virus
ctcacaatcgccttctgcta

SEQ ID NO: 344 - DNA, 20 bp, Human Papilloma Virus
acctcacaatcgccttctgc

SEQ ID NO: 345 - DNA, 22 bp, Human Papilloma Virus
caatcgccttctgctacatata

SEQ ID NO: 346 - DNA, 22 bp, Human Papilloma Virus
acaatcgccttctgctacatat

SEQ ID NO: 347 - DNA, 20 bp, Human Papilloma Virus
ctacatataatagttcagac

SEQ ID NO: 348 - DNA, 23 bp, Human Papilloma Virus
tagtattaagttaactgctgagg

SEQ ID NO: 349 - DNA, 19 bp, Human Papilloma Virus
tcctacagttttagaagag

SEQ ID NO: 350 - DNA, 26 bp, Human Papilloma Virus
gctaacgcctccccccaatggtactt

SEQ ID NO: 351 - DNA, 22 bp, Human Papilloma Virus
ctacagatatgtgcagtcccag

SEQ ID NO: 352 - DNA, 19 bp, Human Papilloma Virus
acctacgcctgataaagca

SEQ ID NO: 353 - DNA, 16 bp, Human Papilloma Virus
ctatgcaaatttaagt

SEQ ID NO: 354 - DNA, 15 bp, Human Papilloma Virus
tgctgttactccatc

SEQ ID NO: 355 - DNA, 20 bp, Human Papilloma Virus
tgctgttactccatctgttg

SEQ ID NO: 356 - DNA, 20 bp, Human Papilloma Virus
actccatctgttgcacaaac

SEQ ID NO: 357 - DNA, 20 bp, Human Papilloma Virus
aaacatttactccagcaaac

SEQ ID NO: 358 - DNA, 23 bp, Human Papilloma Virus
taaaatcactttaactactgaaa

SEQ ID NO: 359 - DNA, 19 bp, Human Papilloma Virus
ttctacaattttagaacag

SEQ ID NO: 360 - DNA, 26 bp, Human Papilloma Virus
attaacattgcccccctccgctagtt

SEQ ID NO: 361 - DNA, 22 bp, Human Papilloma Virus
ctatcgatttgtaaaaaatgca

SEQ ID NO: 362 - DNA, 19 bp, Human Papilloma Virus
ggacagtcctccacaggct

SEQ ID NO: 363 - DNA, 20 bp, Human Papilloma Virus
tctgtgctaccaaaactgtt

SEQ ID NO: 364 - DNA, 15 bp, Human Papilloma Virus
ctaccaaaactgttg

SEQ ID NO: 365 - DNA, 20 bp, Human Papilloma Virus
accaaaactgttgagtctac

SEQ ID NO: 366 - DNA, 22 bp, Human Papilloma Virus
aactgttgagtctacatataaa

SEQ ID NO: 367 - DNA, 20 bp, Human Papilloma Virus
gttgagtctacatataaagc

SEQ ID NO: 368 - DNA, 20 bp, Human Papilloma Virus
ctacatataaagcctctagt

SEQ ID NO: 369 - DNA, 23 bp, Human Papilloma Virus
tgttattaatttaacagctgaaa

SEQ ID NO: 370 - DNA, 19 bp, Human Papilloma Virus
tgctacattactggaggac

SEQ ID NO: 371 - DNA, 20 bp, Human Papilloma Virus
gttcttaccacctcctactg

SEQ ID NO: 372 - DNA, 22 bp, Human Papilloma Virus
ctaccgctttttacagtctcag

SEQ ID NO: 373 - DNA, 22 bp, Human Papilloma Virus
aaacagtcctcctcctgcagaa

SEQ ID NO: 374 - DNA, 16 bp, Human Papilloma Virus
ctatgcagatcttaca

SEQ ID NO: 375 - DNA, 15 bp, Human Papilloma Virus
cagctacatctgctg

SEQ ID NO: 376 - DNA, 20 bp, Human Papilloma Virus
gctacatctgctgctgcaga

SEQ ID NO: 377 - DNA, 22 bp, Human Papilloma Virus
acatctgctgctgcagaataca

SEQ ID NO: 378 - DNA, 20 bp, Human Papilloma Virus
tgctgcagaatacaaggcct

SEQ ID NO: 379 - DNA, 20 bp, Human Papilloma Virus
gctgcagaatacaaggcctc

SEQ ID NO: 380 - DNA, 20 bp, Human Papilloma Virus
cagaatacaaggcctctaac

SEQ ID NO: 381 - DNA, 23 bp, Human Papilloma Virus
taaaatacagttaacaccagaaa

SEQ ID NO: 382 - DNA, 19 bp, Human Papilloma Virus
caaggcactgttggatgat

SEQ ID NO: 383 - DNA, 26 bp, Human Papilloma Virus
tgtgttgccacctccttccaccagtt

SEQ ID NO: 384 - DNA, 22 bp, Human Papilloma Virus
atatcgctttttacagtctcgg

SEQ ID NO: 385 - DNA, 22 bp, Human Papilloma Virus
gggtgctgctgcccctgcgccc

SEQ ID NO: 386 - DNA, 16 bp, Human Papilloma Virus
ttatgccgacatgtca

SEQ ID NO: 387 - DNA, 20 bp, Human Papilloma Virus
atctactgcaactactaatc

SEQ ID NO: 388 - DNA, 15 bp, Human Papilloma Virus
ctgcaactactaatc

SEQ ID NO: 389 - DNA, 20 bp, Human Papilloma Virus
ctgcaactactaatccagtt

SEQ ID NO: 390 - DNA, 22 bp, Human Papilloma Virus
actactaatccagttccatcta

SEQ ID NO: 391 - DNA, 20 bp, Human Papilloma Virus
ctaatccagttccatctata

SEQ ID NO: 392 - DNA, 20 bp, Human Papilloma Virus
ctatatatgaaccttctaaa

SEQ ID NO: 393 - DNA, 23 bp, Human Papilloma Virus
taaaattacacttactactgatg

SEQ ID NO: 394 - DNA, 19 bp, Human Papilloma Virus
tcctactattttagatagt

SEQ ID NO: 395 - DNA, 26 bp, Human Papilloma Virus
tgttagtcctcccccatctgctagct

SEQ ID NO: 396 - DNA, 22 bp, Human Papilloma Virus
atataggtttttacagtcatct

SEQ ID NO: 397 - DNA, 19 bp, Human Papilloma Virus
ggatgtggttgttccacaa

SEQ ID NO: 398 - DNA, 20 bp, Human Papilloma Virus
ctgctgttactcaatctgtt

SEQ ID NO: 399 - DNA, 15 bp, Human Papilloma Virus
tgctgttactcaatc

SEQ ID NO: 400 - DNA, 20 bp, Human Papilloma Virus
gttactcaatctgttgcaca

SEQ ID NO: 401 - DNA, 20 bp, Human Papilloma Virus
tgcacaaacatttactccag

SEQ ID NO: 402 - DNA, 22 bp, Human Papilloma Virus
ttactcaatctgttgcacaaac

SEQ ID NO: 403 - DNA, 20 bp, Human Papilloma Virus
aaacatttactccagcaaac

SEQ ID NO: 404 - DNA, 23 bp, Human Papilloma Virus
taaaatcactttaactactgaaa

SEQ ID NO: 405 - DNA, 19 bp, Human Papilloma Virus
ttctacaattttagaacag

SEQ ID NO: 406 - DNA, 26 bp, Human Papilloma Virus
attaaccttgcccccctcagctagtt

SEQ ID NO: 407 - DNA, 22 bp, Human Papilloma Virus
ctatcgatttgtaaaaaatgca

SEQ ID NO: 408 - DNA, 19 bp, Human Papilloma Virus
ggacagtcctccacaggct

SEQ ID NO: 409 - DNA, 15 bp, Human Papilloma Virus
tgctgctacacaggc

SEQ ID NO: 410 - DNA, 20 bp, Human Papilloma Virus
gctgctacacaggctaatga

SEQ ID NO: 411 - DNA, 20 bp, Human Papilloma Virus
tgctacacaggctaatgaat

SEQ ID NO: 412 - DNA, 22 bp, Human Papilloma Virus
ctacacaggctaatgaatacac

SEQ ID NO: 413 - DNA, 20 bp, Human Papilloma Virus
atgaatacacagcctctaac

SEQ ID NO: 414 - DNA, 23 bp, Human Papilloma Virus
caaaatacatcttacccctgaaa

SEQ ID NO: 415 - DNA, 19 bp, Human Papilloma Virus
tgaacatttattggatgag

SEQ ID NO: 416 - DNA, 26 bp, Human Papilloma Virus
cgtgttaccacctccttccaccagcc

SEQ ID NO: 417 - DNA, 22 bp, Human Papilloma Virus
ctatcgctatctgcagtcccgt

SEQ ID NO: 418 - DNA, 22 bp, Human Papilloma Virus
gggtccttccgcccctgcccct

SEQ ID NO: 419 - DNA, 16 bp, Human Papilloma Virus
ttatgatggccttgta

SEQ ID NO: 420 - DNA, 15 bp, Human Papilloma Virus
tgctaccaacaccga

SEQ ID NO: 421 - DNA, 20 bp, Human Papilloma Virus
ctaccaacaccgaatcagaa

SEQ ID NO: 422 - DNA, 22 bp, Human Papilloma Virus
ccaacaccgaatcagaatataa

SEQ ID NO: 423 - DNA, 20 bp, Human Papilloma Virus
cagaatataaacctaccaat

SEQ ID NO: 424 - DNA, 23 bp, Human Papilloma Virus
taaggtccgtctgactccagagg

SEQ ID NO: 425 - DNA, 19 bp, Human Papilloma Virus
tgactccttattagatgag

SEQ ID NO: 426 - DNA, 26 bp, Human Papilloma Virus
tgttgtgccccctccctccacaagtt

SEQ ID NO: 427 - DNA, 22 bp, Human Papilloma Virus
ctataggtacttgcagtctcgc

SEQ ID NO: 428 - DNA, 22 bp, Human Papilloma Virus
gggggccgccgccgccaagcct

SEQ ID NO: 429 - DNA, 16 bp, Human Papilloma Virus
ttatgctggcatgtcc

SEQ ID NO: 430 - DNA, 409 bp, Human Papilloma Virus subtype 6 (6743-7151/409 bp)
tatttgttggggtaatcaactgtttgttactgtggtagataccacacgcagtaccaacatgacattatgtgcatccgtaactacatcttccacatacaccaattctgattataaagagtacatgcgtcatgtggaagagtatgatttacaatttatttttcaattatgtagcattacattgtctgctgaagtaatggcctatattcacacaatgaatccctctgttttggaagactggaactttgggttatcgcctcccccaaatggtacattagaagatacctataggtatgtgcagtcacaggccattacctgtcaaaagcccactcctgaaaaggaaaagccagatccctataagaaccttagtttttgggaggttaatttaaaagaaaagttttctagtgaattg

SEQ ID NO: 431 - DNA, 409 bp, Human Papilloma Virus subtype 11 (6727-7135/409 bp)
tatttgctggggaaaccacttgtttgttactgtggtagataccacacgcagtacaaatatgacactatgtgcatctgtgtctaaatctgctacatacactaattcagattataaggaatacatgcgccatgtggaggagtttgatttacagtttatttttcaattgtgtagcattacattatctgcagaagtcatggcctatatacacacaatgaatccttctgttttggaggactggaactttggtttatcgcctccaccaaatggtacactggaggatacttatagatatgtacagtcacaggccattacctgtcagaaacccacacctgaaaaagaaaaacaggatccctataaggatatgagtttttgggaggttaacttaaaagaaaagttttcaagtgaatta

SEQ ID NO: 432 - DNA, 412 bp, Human Papilloma Virus subtype 16 (6602-7013/412 bp)
catttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagaccta

SEQ ID NO: 433 - DNA, 415 bp, Human Papilloma Virus subtype 18 (6587-6992/415 bp)
tgtttgctggcataatcaattatttgttactgtggtagataccactcccagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttcccccccccccaactactagtttggtggatacatatcgttttgtacaatctgttgctattacctgtcaaaaggatgctgcaccggctgaaaataaggatccctatgataagttaaagttttggaatgtggatttaaaggaaaagttttctttagactta

SEQ ID NO: 434 - DNA, 415 bp, Human Papilloma Virus subtype 26 (6553-6967/415 bp)
tatctgttggggcaatcaattgtttgttacctgtgttgataccacccgcagtactaaccttaccattagtacattatctgcagcatctgcatccactccatttaaaccatctgattataaacaatttataagacatggcgaagaatatgaattacaatttatatttcagttgtgtaaaataacacttacaacagatgttatggcttacatacatttaatgaatgcctccatattggaggattggaattttggactaaccttacctcccactgctagtttggaagatgcctataggtttattaaaaactctgctactacctgtcagcgtaacgcccctcctgtgccaaaggaagatccttttcaaaaatttaaattttgggatgtagatttaaaagaaaaattttctattgatttg

SEQ ID NO: 435 - DNA, 412 bp, Human Papilloma Virus subtype 31 (6520-6931/412 bp)
tatttgttggggcaatcagttatttgttactgtggtagataccacacgtagtaccaatatgtctgtttgtgctgcaattgcaaacagtgatactacatttaaaagtagtaattttaaagagtatttaagacatggtgaggaatttgatttacaatttatatttcagttatgcaaaataacattatctgcagacataatgacatatattcacagtatgaatcctgctattttggaagattggaattttggattgaccacacctccctcaggttctttggaggatacctataggtttgtcacctcacaggccattacatgtcaaaaaactgccccccaaaagcccaaggaagatccatttaaagattatgtattttgggaggttaatttaaaagaaaagttttctgcagattta

SEQ ID NO: 436 - DNA, 409 bp, Human Papilloma Virus subtype 32 (6837-7245/409 bp)
tatatgttggggtaatcaagtgtttctaactgttgtggatactacccgtagtactaacatgactgtgtgtgctactgtaacaactgaagacacatacaagtctactaactttaaggaatatctacgccatgcagaggaatatgatatacagtttatatttcaattgtgcaaaattacattatctgtagaggttatgtcatatatccacaccatgaatcctgacatactagacgattggaatgttggtgtagctccaccgccctctggtactttagaagatagttatagatttgtgcagtctcaggccatacgatgtcaagctaaggtaacagcacctgaaaaaaaggatcctttttctgactattcattttgggaagtaaatttatctgaaaagttttctagtgattta

SEQ ID NO: 437 - DNA, 409 bp, Human Papilloma Virus subtype 33 (6559-6967/409 bp)
tatttgttggggcaatcaggtatttgttactgtggtagataccactcgcagtactaatatgactttatgcacacaagtaactagtgacagtacatataaaaatgaaaattttaaagaatatataagacatgttgaagaatatgatctacagtttgtttttcaactatgcaaagttaccttaactgcagaagttatgacatatattcatgctatgaatccagatattttagaagattggcaatttggtttaacacctcctccatctgctagtttacaggatacctataggtttgttacctctcaggctattacgtgtcaaaaaacagtacctccaaaggaaaaggaagaccccttaggtaaatatacattttgggaagtggatttaaaggaaaaattttcagcagattta

SEQ ID NO: 438 - DNA, 412 bp, Human Papilloma Virus subtype 35 (6542-6953/412 bp)
tatttgttggagtaaccaattgtttgttactgtagttgatacaacccgtagtacaaatatgtctgtgtgttctgctgtgtcttctagtgacagtacatataaaaatgacaattttaaggaatatttaaggcatggtgaagaatatgatttacagtttatttttcagttatgtaaaataacactaacagcagatgttatgacatatattcatagtatgaacccgtccattttagaggattggaattttggccttacaccaccgccttctggtaccttagaggacacatatcgctatgtaacatcacaggctgtaacttgtcaaaaacccagtgcaccaaaacctaaagatgatccattaaaaaattatactttttgggaggttgatttaaaggaaaagttttctgcagactta

SEQ ID NO: 439 - DNA, 415 bp, Human Papilloma Virus subtype 37 (6711-7125/415 bp)
cattttatggggtaatcaaatgtttatcacagttgctgataatacacggaacacaaacttttctattagtgtgtctactgacaatggcgaagttacagaatataattctcaaacactcagagaatacctaagacatgttgaagaataccagctttcaattattttacaactttgtaaagttcctttaaaggctgaggttttaactcagataaatgcaatgaattctggtatattggaagagtggcaattaggatttgtacctactccagataattcagtacatgacctttataggtacattaattcaaaggctaccaagtgtcctgatgcagttgttgaaaaagaaaaggaagatccctttgcaaaatatacattttggaatgtagatttaactgaaaaattatcattggattta

SEQ ID NO: 440 - DNA, 415 bp, Human Papilloma Virus subtype 39 (6605-7017/415 bp)
tatatgttggcataatcaattatttcttactgttgtggacactacccgtagtaccaactttacattatctacctctatagagtcttccataccttctacatatgatccttctaagtttaaggaatataccaggcacgtggaggagtatgatttacaatttatatttcaactgtgtactgtcacattaacaactgatgttatgtcttatattcacactatgaattcctctatattggacaattggaattttgctgtagctcctccaccatctgccagtttggtagacacttacagatacctacagtctgcagccattacatgtcaaaaggatgctccagcacctgaaaagaaagatccatatgacggtctaaagttttggaatgttgacttaagggaaaagtttagtttggaactt

SEQ ID NO: 441 - DNA, 409 bp, Human Papilloma Virus subtype 42 (6802-7210/409 bp)
tatatgttggggaaatcagctatttttaactgtggttgatactacccgtagtactaacatgactttgtgtgccactgcaacatctggtgatacatatacagctgctaattttaaggaatatttaagacatgctgaagaatatgatgtgcaatttatatttcaattgtgtaaaataacattaactgttgaagttatgtcatatatacacaatatgaatcctaacatattagaggagtggaatgttggtgttgcaccaccaccttcaggaactttagaagatagttataggtatgtacaatcagaagctattcgctgtcaggctaaggtaacaacgccagaaaaaaaggatccttattcagacttttggttttgggaggtaaatttatctgaaaagttttctactgattta

SEQ ID NO: 442 - DNA, 415 bp, Human Papilloma Virus subtype 43 (21-435/415 bp)
catttgttttgggaatcagttgtttgttacagtggtagataccactcgtagtacaaacttgacgttatgtgcctctactgaccctactgtgcccagtacatatgacaatgcaaagtttaaggaatacttgcggcatgtggaagaatatgatctgcagtttatatttcaattatgcataataacgctaaacccagaggttatgacatatattcatactatggatcccacattattagaggactggaattttggtgtgtccccacctgcctctgcttctttggaagatacttatcgctttttgtctaacaaggccattgcatgtcaaaaaaatgctcccccaaaggaacgggaggatccctataaaaagtatacattttgggatataaatcttacagaaaagttttctgcacaactt

SEQ ID NO: 443 - DNA, 415 bp, Human Papilloma Virus subtype 44 (6647-7061/415 bp)
tatttgttggggaaatcagttatttgttactgttgtagatactacccgtagtacaaacatgacaatatgtgctgccactacacagtcccctccgtctacatatactagtgaacaatataagcaatacatgcgacatgttgaggagtttgacttacaatttatgtttcaattatgtagtattaccttaacggcggaggtaatggcctatcttcatactatgaatgctggtattttagaacagtggaactttgggttgtcgccgcccccaaatggtaccttagaggacaaatacagatatgtgcagtcccaggccattacatgtcaaaagccaccccctgaaaaggcaaagcaggacccctatgcaaaattaagtttttgggaggtggatcttagagaaaagttttctagtgagttg

SEQ ID NO: 444 - DNA, 415 bp, Human Papilloma Virus subtype 45 (6582-6996/415 bp)
tatttgttggcataatcagttgtttgttactgtagtggacactacccgcagtactaatttaacattatgtgcctctacacaaaatcctgtgccaagtacatatgaccctactaagtttaagcagtatagtagacatgtggaggaatatgatttacagtttatttttcagttgtgcactattactttaactgcagaggttatgtcatatatccatagtatgaatagtagtatattagaaaattggaattttggtgtccctccaccacctactacaagtttggtggatacatatcgttttgtgcaatcagttgctgttacctgtcaaaaggatactacacctccagaaaagcaggatccatatgataaattaaagttttggactgttgacctaaaggaaaaattttcctccgatttg

SEQ ID NO: 445 - DNA, 412 bp, Human Papilloma Virus subtype 51 (6486-6897/412 bp)
catttgctggaacaatcagctttttattacctgtgttgatactaccagaagtacaaatttaactattagcactgccactgctgcggtttccccaacatttactccaagtaactttaagcaatatattaggcatggggaagagtatgaattgcaatttatttttcaattatgtaaaattactttaactacagaggtaatggcttatttacacacaatggatcctaccattcttgaacagtggaattttggattaacattacctccgtctgctagtttggaggatgcatataggtttgttagaaatgcagctactagctgtcaaaaggacacccctccacaggctaagccagatcctttggccaaatataaattttgggatgttgatttaaaggaacgattttctttagattta

SEQ ID NO: 446 - DNA, 409 bp, Human Papilloma Virus subtype 52 (6623-7031/409 bp)
catatgttggggcaatcagttgtttgtcacagttgtggataccactcgtagcactaacatgactttatgtgctgaggttaaaaaggaaagcacatataaaaatgaaaattttaaggaataccttcgtcatggcgaggaatttgatttacaatttatttttcaattgtgcaaaattacattaacagctgatgttatgacatacattcataagatggatgccactattttagaggactggcaatttggccttaccccaccaccgtctgcatctttggaggacacatacagatttgtcacttctactgctataacttgtcaaaaaaacacaccacctaaaggaaaggaagatcctttaaaggactatatgttttgggaggtggatttaaaagaaaagttttctgcagattta

SEQ ID NO: 447 - DNA, 409 bp, Human Papilloma Virus subtype 53 (6614-7022/409 bp)
catctgttggaacaatcagttatttgtaactgttgtggataccaccaggaatacaaacatgactctttccgcaaccacacagtctatgtctacatataattcaaagcaaattaaacagtatgttagacatgcagaggaatatgaattacaatttgtgtttcaactatgtaaaatatccctgtctgctgaggttatggcctatttacatactatgaattctaccttactggaagactggaatataggtttgtcgcctcctgttgccactagcttagaggacaaatacagatatgtgaaaagtgcagctataacctgtcaaaaggatcagccccctcctgaaaagcaggacccactatctaaatataaattttgggaggtcaatttgcaaaacagtttttctgctgatttg

SEQ ID NO: 448 - DNA, 412 bp, Human Papilloma Virus subtype 54 (6561-6972/412 bp)
tatttgttggggcaatcaggtgtttttaacagttgtagataccacccgtagtactaacctaacattgtgtgctacagcatccacgcaggatagctttaataattctgactttagggagtatattagacatgtggaggaatatgatttacagtttatatttcagttatgtaccataacccttacagcagatgttatggcctatattcatggaatgaatcccactattctagaggactggaactttggtataacccccccagctacaagtagtttggaggacacatataggtttgtacagtcacaggccattgcatgtcaaaagaataatgcccctgcaaaggaaaaggaggatccttacagtaaatttaatttttggactgttgaccttaaggaacgattttcatctgacctt

SEQ ID NO: 449 - DNA, 415 bp, Human Papilloma Virus subtype 55 (6647-7061/415 bp)
tatttgttgggggaatcagttatttgttactgttgtagatactacacgtagtacaaacatgacaatatgtgctgctacaactcagtctccatctacaacatataatagtacagaatataaacaatacatgcgacatgttgaggagtttgacttacagtttatgtttcaattatgtagtattaccttaactgctgaggtaatggcctatttacataccatgaatcctggtattttggaacagtggaactttgggttgtcgccacccccaaatggtaccttagaagacaaatacagatatgtgcagtcacaggccattacatgtcaaaagcctccccctgaaaaggcaaagcaggacccctatgcaaaattaagtttttgggaggtagatctcagagaaaagttttctagtgagtta

SEQ ID NO: 450 - DNA, 409 bp, Human Papilloma Virus subtype 56 (6559-6967/409 bp)
catttgctggggtaatcaattatttgttactgtagtagatactactagaagtactaacatgactattagtactgctacagaacagttaagtaaatatgatgcacgaaaaattaatcagtaccttagacatgtggaggaatatgaattacaatttgtttttcaattatgcaaaattactttgtctgcagaggttatggcatatttacataatatgaatgctaacctactggaggactggaatattgggttatccccgccagtggccaccagcctagaagataaatatagatatgttagaagcacagctataacatgtcaacgggaacagccaccaacagaaaaacaggacccattagctaaatataaattttgggatgttaacttacaggacagtttttctacagacctg

SEQ ID NO: 451 - DNA, 409 bp, Human Papilloma Virus subtype 58 (6608-7016/409 bp)
catttgctggggcaatcagttatttgttaccgtggttgataccactcgtagcactaatatgacattatgcactgaagtaactaaggaaggtacatataaaaatgataattttaaggaatatgtacgtcatgttgaagaatatgacttacagtttgtttttcagctttgcaaaattacactaactgcagagataatgacatatatacatactatggattccaatattttggaggactggcaatttggtttaacacctcctccgtctgccagtttacaggacacatatagatttgttacctcccaggctattacttgccaaaaaacagcaccccctaaagaaaaggaagatccattaaataaatatactttttgggaggttaacttaaaggaaaagttttctgcagatcta

SEQ ID NO: 452 - DNA, 415 bp, Human Papilloma Virus subtype 59 (6571-6985/415 bp)
tatatgttggcacaatcaattgtttttaacagttgtagatactactcgcagcaccaatctttctgtgtgtgcttctactacttcttctattcctaatgtatacacacctaccagttttaaagaatatgccagacatgtggaggaatttgatttgcagtttatatttcaactgtgtaaaataacattaactacagaggtaatgtcatacattcataatatgaataccactattttggaggattggaattttggtgttacaccacctcctactgctagtttagttgacacataccgttttgttcaatctgctgctgtaacttgtcaaaaggacaccgcaccgccagttaaacaggacccttatgacaaactaaagttttggcctgtagatcttaaggaaaggttttctgcagatctt

SEQ ID NO: 453 - DNA, 415 bp, Human Papilloma Virus subtype 61 (6732-7146/415 bp)
tatttgttggtttaatgaattgtttgtaaccgttgtggataccacccgcagtactaatttaaccatttgtactgctacatccccccctgtatctgaatataaagccacaagctttagggaatatttgcgccatacagaggagtttgatttgcaatttatttttcagttatgtaaaatacatttaacccctgaaattatggcctacctacataatatgaataaggccttgttggatgactggaactttggtgtggtaccaccaccctctaccagtttagaagacacatataggtttttgcagtccagagctattacatgtcagaagggtgctgctgccccgccgcccaaggaggatcgctatgccaagttatccttttggactgttgatttacgagacaagttttccactgatttg

SEQ ID NO: 454 - DNA, 409 bp, Human Papilloma Virus subtype 62 (21-429/409 bp)
tatttgttggtttaatgaactgtttgttactgtggtggatactaccagaagtactaattttactatttgtaccgcctccactgctgcagcagaatacacggctaccaactttagggaatttttgcgacacacggaggaatttgatttgcaatttatatttcaattgtgcaaaatacagttaacccccgaaattatggcctacctgcataatatgaacaaggaccttttggatgactggaactttggggttttacctcccccttccactagtttagatgagacatatcactatttcgagtctcgggctattacatgtcaaagggggctgcctacccgtcccaaggtggacccgtatgcgcaaatgacattttggactgtggatcttaaggacaagttgtctactgatttg

SEQ ID NO: 455 - DNA, 409 bp, Human Papilloma Virus subtype 66 (6609-7017/409 bp)
catatgctggggtaatcaggtatttgttactgttgtggatactaccagaagcaccaacatgactattaatgcagctaaaagcacattaactaaatatgatgcccgtgaaatcaatcaataccttcgccatgtggaggaatatgaactacagtttgtgtttcaactttgtaaaataaccttaactgcagaagttatggcatatttgcataatatgaataatactttattagacgattggaatattggcttatccccaccagttgcaactagcttagaggataaatataggtatattaaaagcacagctattacatgtcagagggaacagccccctgcagaaaagcaggatcccctggctaaatataagttttgggaagttaatttacaggacagcttttctgcagacctg

SEQ ID NO: 456 - DNA, 409 bp, Human Papilloma Virus subtype 67 (6584-6992/409 bp)
tatatgctggggtaatcaaatatttgttactgttgtagacactacacgtagtaccaacatgactttatgttctgaggaaaaatcagaggctacatacaaaaatgaaaactttaaggaataccttagacatgtggaagaatatgatttgcagtttatatttcagctgtgcaaaatatcccttactgcaaatgttatgcaatacatacacaccatgaatccagatatattagaggactggcaatttggccttacaccacctccttcaggtaatttacaggacacatatagatttgttacctcgcaggctattacctgtcaaaaaacatcccctccaacagcaaaggaagatcctcttaaaaagtacagtttttgggaaatcaatttaaaggaaaaattttctgcagattta

SEQ ID NO: 457 - DNA, 415 bp, Human Papilloma Virus subtype 68 (2582-2996/415 bp)
tatttgttggcataatcaattatttcttactgttgtggataccactcgcagtaccaattttactttgtctactactactgaatcagctgtaccaaatatttatgatcctaataaatttaaggaatatattaggcatgttgaggaatatgatttgcaatttatatttcagttgtgtactataacattgtccactgatgtaatgtcctatatacatactatgaatcctgctattttggatgattggaattttggtgttgcccctccaccatctgctagtcttgtagatacataccgctatctgcaatcagcagcaattacatgtcaaaaagacgcccctgcacctactaaaaaggatccatatgatggcttaaacttttggaatgtaaatttaaaggaaaagtttagttctgaactg

SEQ ID NO: 458 - DNA, 415 bp, Human Papilloma Virus subtype 69 (6509-6923/415 bp)
catttgttggggcaaccaattgtttgttacttgtgtagatactacccgcagtaccaacctcactattagtactgtatctgcacaatctgcatctgccacttttaaaccatcagattataagcagtttataaggcatggtgaggaatatgaattacagtttatatttcaattgtgtaaaattactcttaccactgatgtaatggcctatatccatacaatgaattctactattttggaaaattggaattttggccttaccttgcctcctactgctagtttggaagatgcatataggtttattaaaaattcagctactacatgtcaacgcgatgcccctgcacagcccaaggaggatccatttagtaaattaaaattttgggacgttgatcttaaagaaaagttttctattgattta

SEQ ID NO: 459 - DNA, 415 bp, Human Papilloma Virus subtype 70 (6549-6963/415 bp)
catttgttggcataaccagttgtttattactgtggtggacactacacgtagtactaattttacattgtctgcctgcaccgaaacggccatacctgctgtatatagccctacaaagtttaaggaatatactaggcatgtggaggaatatgatttacaatttatatttcaattgtgtactatcacattaactgctgacgttatggcctacatccatactatgaatcctgcaattttggacaattggaatataggagttacccctccaccatctgcaagcttggtggacacgtataggtatttacaatcagcagctatagcatgtcaaaaggatgctcctacacctgaaaaaaaggatccctatgacgatttaaaattttggaatgttgatttaaaggaaaagtttagtacagaacta

SEQ ID NO: 460 - DNA, 415 bp, Human Papilloma Virus subtype 72 (6758-7172/415 bp)
catctgttggtttaatgagctttttgtgacagttgtagatactactcgcagtactaatgtaactatttgtactgccacagcgtcctctgtatcagaatatacagcttctaattttcgtgagtatcttcgccacactgaggaatttgatttgcagtttatatttcaactgtgtaaaattcacttaactcctgaaattatggcctacttgcacaatatgaataaggccttattggatgactggaattttggtgtggtgcctcctccttctaccagtttggatgatacctataggtttttgcagtctcgtgccattacctgtcaaaagggggctgccacccctcctcctaaagaagatccatatgctaacttatccttttggactgtggatttaaaggacaaattttccactgacttg

SEQ ID NO: 461 - DNA, 415 bp, Human Papilloma Virus subtype 74 (1613-2027/415 bp)
tatttgttggggtaatcaattatttgttacagttgtggataccacacgcagtactaacatgactgtgtgtgctcctacctcacaatcgccttctgctacatataatagttcagactacaaacaatacatgcgacatgtggaggaatttgatttgcaatttatttttcaattatgtagtattaagttaactgctgaggttatggcctatattcatactatgaatcctacagttttagaagagtggaactttgggctaacgcctccccccaatggtactttagaagacacctacagatatgtgcagtcccaggctattacatgtcaaaaacctacgcctgataaagcaaagcccaatccctatgcaaatttaagtttttgggaagttaatcttaaggaaaagttttctagtgaatta

SEQ ID NO: 462 - DNA, 415 bp, Human Papilloma Virus subtype 82 (6536-6950/415 bp)
catttgctggaataatcagctttttattacttgtgttgacactactaaaagtaccaatttaaccattagcactgctgttactccatctgttgcacaaacatttactccagcaaactttaagcagtacattaggcatggggaagaatatgaattgcaatttatatttcaattgtgtaaaatcactttaactactgaaattatggcttacctgcacaccatggattctacaattttagaacagtggaattttggattaacattgcccccctccgctagtttggaggatgcctatcgatttgtaaaaaatgcagcaacatcctgtcacaaggacagtcctccacaggctaaagaagaccctttggcaaaatataaattttggaatgtagaccttaaggaacgcttttctttggatttg

SEQ ID NO: 463 - DNA, 412 bp, Human Papilloma Virus subtype CP8061 (21-432/412 bp)
catttgttggggcaatcagctttttgtaacagttgtggacacatcacgtagtacaaatatgtccatctgtgctaccaaaactgttgagtctacatataaagcctctagtttcatggaatatttgagacatggagaagaatttgatttgcaatttatatttcaactatgtgttattaatttaacagctgaaattatggcctacttacatcgcatggatgctacattactggaggactggaatttttggttcttaccacctcctactgctagtcttggtgatacctaccgctttttacagtctcaggccataacctgtcagaaaaacagtcctcctcctgcagaaaaaaaggacccctatgcagatcttacattttgggaggtggatttaaaggagcggttttcactagaattg

SEQ ID NO: 464 - DNA, 412 bp, Human Papilloma Virus subtype CP8304 (21-432/412 bp)
tatttgttggtttaatgaaatgtttgttacagtggtggatactaccagaagcaccaattttactatttgcacagctacatctgctgctgcagaatacaaggcctctaactttaaggaatttctgcgccatacagaggaatatgatttgcagtttattttccaattatgtaaaatacagttaacaccagaaattatggcctacttacataatatgaacaaggcactgttggatgattggaattttggtgtgttgccacctccttccaccagtttagatgacacatatcgctttttacagtctcgggccattacctgtcaaaagggtgctgctgcccctgcgcccaaagaggacccttatgccgacatgtcattttggacagttgaccttaaggacaagttgtctactgatttg

SEQ ID NO: 465 - DNA, 350 bp, Human Papilloma Virus subtype L1AE5 (11-360/350 bp)
ggcacaaccaattatttataactgtggtagacacaacacgtagtaccaatcttaccttatctactgcaactactaatccagttccatctatatatgaaccttctaaatttaaggaatacacacgccatgtagaggaatatgatttacaatttatatttcaattgtgtaaaattacacttactactgatgttatgtcttatatacataacatggatcctactattttagatagttggaattttggtgttagtcctcccccatctgctagcttagtagatacatataggtttttacagtcatctgccattacatgtcagaaggatgtggttgttccacaaaaaaaggatcca

SEQ ID NO: 466 - DNA, 415 bp, Human Papilloma Virus subtype MM4 (21-435/415 bp)
catttgctggaataatcagctttttattacttgtgttgacactactagaagtaccaatttaaccattagcactgctgttactcaatctgttgcacaaacatttactccagcaaactttaagcaatacattaggcatggggaagaatatgaattgcaatttatatttcaattgtgtaaaatcactttaactactgaaattatggcttacctgcacaccatggattctacaattttagaacagtggaattttggattaaccttgcccccctcagctagtttggaggatgcctatcgatttgtaaaaaatgcagcaacatcctgtcacaaggacagtcctccacaggctaaacaagaccctttggcaaaatataaattttggaatgtagaccttaaggaacgcttttctttggatttg

SEQ ID NO: 467 - DNA, 412 bp, Human Papilloma Virus subtype MM7 (21-432/412 bp)
catttgttggtttaatgagttatttgttacagttgtagatactacccgcagtaccaatattactatttcagctgctgctacacaggctaatgaatacacagcctctaactttaaggaatacctccgccacaccgaggaatatgacttacaggttatattgcaactttgcaaaatacatcttacccctgaaattatggcatacctacatagtatgaatgaacatttattggatgagtggaattttggcgtgttaccacctccttccaccagccttgatgatacctatcgctatctgcagtcccgtgctattacctgccaaaagggtccttccgcccctgcccctaaaaaggatccttatgatggccttgtattttgggaggttgatttaaaggacaaactatccacagatttg

SEQ ID NO: 468 - DNA, 412 bp, Human Papilloma Virus subtype MM8 (21-432/412 bp)
tatatgctggtttaatcaattgtttgtcacggtggtggataccacccgcagcaccaattttactattagtgctgctaccaacaccgaatcagaatataaacctaccaattttaaggaatacctaagacatgtggaggaatatgatttgcagtttatattccagttgtgtaaggtccgtctgactccagaggtcatgtcctatttacatactatgaatgactccttattagatgagtggaattttggtgttgtgccccctccctccacaagtttagatgatacctataggtacttgcagtctcgcgccattacttgccaaaagggggccgccgccgccaagcctaaggaagatccttatgctggcatgtccttttgggatgtagatttaaaggacaagttttctactgatttg

SEQ ID NO: 469 - DNA, 15 bp, Artificial Sequence
  Synthetic oligonucleotide
ttttttttttttttt

SEQ ID NO: 470 - DNA, 20 bp, Artificial Sequence
  Synthetic oligonucleotide
gcccagactgtgggtggcag

SEQ ID NO: 471 - DNA, 21 bp, Artificial Sequence
  Synthetic oligonucleotide
ccaccaactgcttagcacccc

SEQ ID NO: 472 - DNA, 21 bp, Artificial Sequence
  Synthetic oligonucleotide
tgcagcgtactccccacatca

(19) United States
(12) Patent Application Publication
(10) Pub. No.: US 2007/0031827 A1
(43) Pub. Date: Feb. 8, 2007

(54) Method and detector for identifying subtypes of human papilloma viruses

(76) Inventors: Ching-Yu Lin, Ilan City (TW); Ruey-Wen Lin, Taipei (TW); Chiou-Mien You, Ilan City (TW); Hsing-Hsuan Huang, Ilan (TW); Bor-Heng Lee, Chiai (TW); Hsien-Hsiung Lee, Taipei (TW); Yu-Ju Lin, Ilan City (TW); Chih-Chun Fan, Miaoli (TW); Han-Chuan Hsu, Ilan (TW); Chia-Wen Shih, Taichung (TW); Chih-Hsing Yeh, Yunlin (TW); Yi-Feng Kao, Sanchung City (TW); Chih-Long Pan, Ilan City (TW); Peter Chan, Taipei (TW)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/520,354

(22) Filed: Sep. 12, 2006

Related U.S. Application Data
(62) Division of application No. 10/601,497, filed on Jun. 23, 2003.
Application No. 10/601,497 is a continuation-in-part of application No. 09/885,799, filed on Jun. 20, 2001, now abandoned.

(51) Int. Cl. C12Q 1/70; C12Q 1/68; C12P 19/34

(52) U.S. Cl. 435/5; 435/6; 435/91.2

(57) ABSTRACT

A detector for detecting and simultaneously diagnosing at least one subtype of human papilloma viruses (HPV) contained in a biological sample is provided. The detector comprises: a carrier, a plurality of micro-dots immobilized on the carrier, wherein each micro-dot is for identifying one particular HPV subtype, and the HPV subtype is one selected from a group consisting of 39 different HPV subtypes; and at least one oligonucleotide sequence contained in each the micro-dot that is specific to the one particular HPV subtype, wherein the at least one oligonucleotide sequence serves as a detection probe that hybridizes specifically with an L1 gene sequence of the one particular HPV subtype to form a hybridization complex as a detection indicator, so that each micro-dot identifies one particular HPV subtype via a corresponding oligonucleotide of the one particular HPV subtype, and thereby detecting and simultaneously identifying subtypes of human papilloma viruses.

### CROSS REFERENCE TO RELATED APPLICATION

This application is a divisional of U.S. patent application Ser. No. 10/601,497, filed Jun. 23, 2003 which is a continuation in part of U.S. patent application Ser. No. 09/885,799 filed Jun. 20, 2001, now abandoned, the entirety of which is hereby incorporated by reference into this application.

### FIELD OF THE INVENTION

The present invention relates to a method and a detector for detecting human papilloma viruses, and more particularly to a method and a detector for simultaneously detecting and identifying subtype of human papilloma viruses (HPV).

### BACKGROUND OF THE INVENTION

In humans, more than 70 genetically distinct strains of human papilloma virus (HPV) have been identified based on DNA hybridization studies. According to some reports, different HPV types cause distinct diseases. For example, “Low-risk” HPVs, e.g., HPV 6 and HPV 11, cause benign hyperplasias such as genital warts, while “high-risk” HPVs, e.g., HPV-16, HPV-18, HPV-31, HPV-33, HPV-54, and the like, can cause cancers such as cervical or penile carcinoma.

Cervical cancer is the most common cancer in women. The consorts are often men with penile warts. Sexual activity appears to be an important predisposing factor of the epidemic disease and precancerous lesions. In early 5 to 10 years during the development of cervical cancer, cervical cells form cervical intraepithelial neoplasm.

Recently, in order to decrease the incidence of cervical cancer, Pap smear is used for the cervical cancer screening. However, the Pap smear has a false negative rate of about 30%˜40%. In addition, it is known that more that 95% of cervical carcinoma tissue contain detectable DNA sequences for known varieties of the human papilloma virus (HPV). Hence, the combination of Pap smear and HPV detection for the cervical cancer screening is necessarily considered.

The Applicant cooperates with the hospital to do the epidemiological research in women cervical cancer by using Pap smear and HPV detection, wherein the HPV detection is proceeded by using polymerase chain reaction and nucleotide sequencing. There are 2424 women aged from 16 to 84 for the epidemiology research, wherein 1963 women provide the effective specimen. The research results are shown as follows.
1) 1.9% (37/1963) of the women have abnormal cytological smears.
2) 12.7% (244/1926) of the women with normal cytological smears but have HPV infection.
3) The HPV prevalence in the women with abnormal cytological smears is 51.4% (19/37) and positively relative to the degree of the abnormal cytological smears, wherein the incidence of abnormal non-typical squamous cells is 23.1%, the incidence of low abnormal epithelial cells is 41.7%, and the incidence of high abnormal epithelial cells is 75%.
4) The subtypes of human papilloma viruses detected in the specimens are HPV 52, HPV 58, HPV 70, HPV 16, HPV 18, HPV 68, HPV 33, HPV 66, HPV 35, HPV 37, HPV 54, HPV 59, HPV 67, HPV 72, HPV 69, HPV 82, HPV 39, HPV 31, HPV 32, HPV HLT7474-S, HPV 6, HPV CP8061, HPV 62, HPV CP8304, HPV 44, HPV 11, HPV 61, HPV 74, HPV 42 and HPV 43.

The conventional HPV detecting kits are only used for detecting 18 subtypes of human papilloma viruses including high risk HPV 16, HPV 18, HPV 31, HPV 33, HPV 35, HPV 39, HPV 45, HPV 51, HPV 52, HPV 56, HPV 58, HPV 59 and HPV 68, and detecting low risk HPV 6, HPV I1, HPV 42, HPV 43 and HPV 44.

However, according to the comparison of the epidemiology research and the conventional HPV detecting kits, several clinically-important subtypes of human papilloma viruses contained in a specimen could not be identified by the conventional HPV detecting kits. In addition, the conventional HPV detecting kits only tell the information of HPVs contained in a specimen by two categories, high risk HPVs or low HPVs, rather than tell the definite subtypes as which they are classified. Therefore, except the high risk HPVs and the low risk HPVs, if other HPV subtypes are contained in the specimen, the conventional HPV detecting kits can not identify immediately, which would seriously affects the diagnosis accuracy. Furthermore, the conventional HPV detecting kits lack the system control for checking the house-keeping genes contained in a specimen. Without the system control, it will be hard to confirm whether the detecting protocols are precisely followed. That is, the user can not tell the positive/negative result comes from the HPV subtypes presence/absence or comes from the incorrect protocols execution. Therefore, the conventional detecting kit without the system control would not be able to provide a convincing result.

From the above description, it is known that the conventional detecting kit can not identify many HPV subtypes at the same time and it does not include an internal control in the detecting system. Therefore, how to simultaneously detect many HPV subtypes contained in a biological simple and design an accurate internal control in the detecting kits have become a major problem waited to be solved. In order to overcome the foresaid drawbacks of the conventional HPV detecting kits, the present invention provides a method and a detector for simultaneously detecting and identifying subtypes of human papilloma viruses contained in a sample.

### SUMMARY OF THE INVENTION

It is therefore an object of the present invention to provide a detector for simultaneously detecting and identifying subtypes of human papilloma viruses (HPV) contained in a sample.

The main purpose of the present invention is to provide a HPV detecting kit, which is able to diagnose multiple HPV subtypes (up to 39 different subtypes) at the same time, allowing the rapid and reliable detection and identification of HPV possibly present in a biological sample.

It is another object of the present invention to provide a rapid and reliable method to detect and identify the HPV present in a biological sample.

It is another object of the present invention to provide a HPV detecting kit with high specificity and accuracy, which includes an internal control to show whether the detecting process is well handled so that the detecting result is dependable.

It is another object of the present invention to provide a number of oligonucleotides as probes for detecting and identifying the HPV present in a biological sample.

According to one aspect of the present invention, a detector for detecting and simultaneously diagnosing at least one subtype of human papilloma viruses (HPV) contained in a biological sample, comprises: a carrier, a plurality of micro-dots immobilized on the carrier, wherein each micro-dot is for identifying one particular HPV subtype, and the HPV subtype is one selected from a group consisting of (HPV 6, HPV 1, HPV 16, HPV 18, HPV 26, HPV 31, HPV 32, HPV 33, HPV 35, HPV 37, HPV 39, HPV 42, HPV 43, HPV 44, HPV 45, HPV 51, HPV 52, HPV 53, HPV 54, HPV 55, HPV 56, HPV 58, HPV 59, HPV 61, HPV 62, HPV 66, HPV 67, HPV 68, HPV 69, HPV 70, HPV 72, HPV 74, HPV 82, HPV CP8061, HPV CP8034, HPV LIAE5, HPV MM4, HPV MM7 and HPV MM8); and at least one oligonucleotide sequence contained in each the micro-dot that is specific to the one particular HPV subtype, wherein the at least one oligonucleotide sequence serves as a detection probe that hybridizes specifically with an LI gene sequence of the one particular HPV subtype to form a hybridization complex as a detection indicator, so that each micro-dot identifies one particular HPV subtype via a corresponding oligonucleotide of the one particular HPV subtype, and thereby detecting and simultaneously identifying subtypes of human papilloma viruses.

In accordance with the present invention, the at least one oligonucleotide that hybridizes specifically with an LI gene sequence of the one particular HPV subtype is respectively chosen from the following list for each HPV subtype: (SEQ ID NO: 1-SEQ ID NO: 12) for HPV 6, (SEQ ID NO: 13-SEQ ID NO:24) for HPV 11, (SEQ ID NO:25-SEQ ID NO:36) for HPV 16, (SEQ ID NO:37-SEQ ID NO:48) for HPV 18, (SEQ ID NO:49-SEQ ID NO:58) for HPV 26, (SEQ ID NO:59-SEQ ID NO:68) for HPV 31, (SEQ ID NO:69-SEQ ID NO:79) for HPV 32, (SEQ ID NO:80-SEQ ID NO:90) for HPV 33, (SEQ ID NO:91-SEQ ID NO:100) for HPV 35, (SEQ ID NO:101-SEQ ID NO:1 12) for HPV 37, (SEQ ID NO:113-SEQ ID NO:123) for HPV 39, (SEQ ID NO: 124-SEQ ID NO: 133) for HPV 42, (SEQ ID NO: 134-SEQ ID NO: 143) for HPV 43, (SEQ ID NO: 144-SEQ ID NO: 154) for HPV 44, (SEQ ID NO: 155-SEQ ID NO: 165) for HPV 45, (SEQ ID NO: 166-SEQ ID NO: 177) for HPV 51, (SEQ ID NO:178-SEQ ID NO:189) for HPV 52, (SEQ ID NO:190-SEQ ID NO:199) for HPV 53, (SEQ ID NO:200-SEQ ID NO:209) for HPV 54, (SEQ ID NO:210-SEQ ID NO:218) for HPV 55, (SEQ ID NO:219-SEQ ID NO:228) for HPV 56, (SEQ ID NO:229-SEQ ID NO:239) for HPV 58, (SEQ ID NO:240-SEQ ID NO:250) for HPV 59, (SEQ ID NO:251-SEQ ID NO:261) for HPV 61, (SEQ ID NO:262-SEQ ID NO:272) for HPV 62, (SEQ ID NO:273-SEQ ID NO:283) for HPV 66, (SEQ ID NO:284-SEQ ID NO:294) for HPV 67, (SEQ ID NO:295-SEQ ID NO:305) for HPV 68, (SEQ ID NO:306-SEQ ID NO:316) for HPV 69, (SEQ ID NO:317-SEQ ID NO:328) for HPV 70, (SEQ ID NO:329-SEQ ID NO:341) for HPV 72, (SEQ ID NO:342-SEQ ID NO:353) for HPV 74, (SEQ ID NO:354-SEQ ID NO:362) for HPV 82, (SEQ ID NO:363-SEQ ID NO:374) for HPV CP8061, (SEQ ID NO:375-SEQ ID NO:386) for HPV CP8034, (SEQ ID NO:387-SEQ ID NO:397) for HPV L1AE5, (SEQ ID NO:398-SEQ ID NO:408) for HPV MM4, (SEQ ID NO:409-SEQ ID NO:419) for HPV MM7, and (SEQ ID NO:420-SEQ ID NO:429) for HPV MM8.

Preferably, the carrier is a nylon membrane.

Preferably, the carrier is a glass plate.

Preferably, the detector is an oligonucleotide biochip.

Preferably, the at least one oligonucleotide has a length between 15-30 bases.

Preferably, the detector further comprises a micro-dot containing a Glutaldehyde-3-phosphodehydrogenase (GAPDH) gene, which is used as an internal control.

According to another aspect of the present invention, a method for detecting and simultaneously diagnosing at least one subtype of human papilloma viruses (HPV) contained in a biological sample is provided. The detecting method comprises steps of: amplifying an L1 gene fragment of human papilloma viruses (HPV) contained in the biological sample and obtaining an amplification product by polymerase chain reaction (PCR) using primers labeled with signaling substance; hybridizing the amplification product with a detector according to claim 1 to form a hybridization complex; removing nonhybridized the amplification product; and detecting the hybridization complex through detecting the signaling substance, thereby detecting and simultaneously identifying HPV subtypes contained in the biological sample.

Preferably, the amplification product has a length of 450 base pairs by using MY09 as sense primer and MY11 as anti-sense primer in polymerase chain reaction (PCR).

Preferably, the amplification product has a length of 190 base pairs by using MY11 as sense primer and GP6+ as anti-sense primer in polymerase chain reaction (PCR).

Preferably, the signaling substance is biotin.

Preferably, the biotin reacts with avidin-alkalinephosphatase to show the hybridization result by presenting a particular color.

Preferably, the signaling substance is a fluorescent substance.

Preferably, the fluorescent substance is Cyanine 5.

According to another aspect of the present invention, a probe which hybridizes to nucleic acid from an HPV subtype, the probe being selected from the group consisting of: SEQ ID NO:1-SEQ ID NO:12 and sequences fully complementary thereto, which hybridize with HPV 6; SEQ ID NO:13-SEQ ID NO:24 and sequences fully complementary thereto, which hybridize with HPV 11; SEQ ID NO:25-SEQ ID NO:36 and sequences fully complementary thereto, which hybridize with HPV 16; SEQ ID NO:37-SEQ ID NO:48 and sequences fully complementary thereto, which hybridize with HPV 18; SEQ ID NO:49-SEQ ID NO:58 and sequences fully complementary thereto, which hybridize with HPV 26; SEQ ID NO:59-SEQ ID NO:68 and sequences fully complementary thereto, which hybridize with HPV 31; SEQ ID NO:69-SEQ ID NO:79 and sequences fully complementary thereto, which hybridize with HPV 32; SEQ ID NO:80-SEQ ID NO:90 and sequences fully complementary thereto, which hybridize with HPV 33; SEQ ID NO:91-SEQ ID NO:100 and sequences fully complementary thereto, which hybridize with HPV 35; SEQ ID NO:101-SEQ ID NO:112 and sequences fully complementary thereto, which hybridize with HPV 37; SEQ ID NO:113-SEQ ID NO: 123 and sequences fully complementary thereto, which hybridize with HPV 39; SEQ ID NO: 124-SEQ ID NO:133 and sequences fully complementary thereto, which hybridize with HPV 42; SEQ ID NO:1 34-SEQ ID NO:143 and sequences fully complementary thereto, which hybridize with HPV 43; SEQ ID NO:144-SEQ ID NO:154 and sequences fully complementary thereto, which hybridize with HPV 44; SEQ ID NO:155-SEQ ID NO:165 and sequences fully complementary thereto, which hybridize with HPV 45; SEQ ID NO:166-SEQ ID NO:177 and sequences fully complementary thereto, which hybridize with HPV 51; SEQ ID NO:178-SEQ ID NO:189 and sequences fully complementary thereto, which hybridize with HPV 52; SEQ ID NO:190-SEQ ID NO:199 and sequences fully complementary thereto, which hybridize with HPV 53; SEQ ID NO:200-SEQ ID NO:209 and sequences fully complementary thereto, which hybridize with HPV 54; SEQ ID NO:210-SEQ ID NO:218 and sequences fully complementary thereto, which hybridize with HPV 55; SEQ ID NO:219-SEQ ID NO:228 and sequences fully complementary thereto, which hybridize with HPV 56; SEQ ID NO:229-SEQ ID NO:239 and sequences fully complementary thereto, which hybridize with HPV 58; SEQ ID NO:240-SEQ ID NO:250 and sequences fully complementary thereto, which hybridize with HPV 59; SEQ ID NO:251-SEQ ID NO:261 and sequences fully complementary thereto, which hybridize with HPV 61; SEQ ID NO:262-SEQ ID NO:272 and sequences fully complementary thereto, which hybridize with HPV 62; SEQ ID NO:273-SEQ ID NO:283 and sequences fully complementary thereto, which hybridize with HPV 66; SEQ ID NO:284-SEQ ID NO:294 and sequences fully complementary thereto, which hybridize with HPV 67; SEQ ID NO:295-SEQ ID NO:305 and sequences fully complementary thereto, which hybridize with HPV 68; SEQ ID NO:306-SEQ ID NO:316 and sequences fully complementary thereto, which hybridize with HPV 69; SEQ ID NO:317-SEQ ID NO:328 and sequences fully complementary thereto, which hybridize with HPV 70; SEQ ID NO:329-SEQ ID NO:341and sequences fully complementary thereto, which hybridize with HPV 72; SEQ ID NO:342-SEQ ID NO:353 and sequences fully complementary thereto, which hybridize with HPV 74; SEQ ID NO:354-SEQ ID NO:362 and sequences fully complementary thereto, which hybridize with HPV 82; SEQ ID NO:363-SEQ ID NO:374 and sequences fully complementary thereto, which hybridize with HPV CP8061; SEQ ID NO:375-SEQ ID NO:386 and sequences fully complementary thereto, which hybridize with HPV CP8034; SEQ ID NO:387-SEQ ID NO:397 and sequences fully complementary thereto, which hybridize with HPV L1AE5; SEQ ID NO:398-SEQ ID NO:408 and sequences fully complementary thereto, which hybridize with HPV MM4; SEQ ID NO:409-SEQ ID NO:419 and sequences fully complementary thereto, which hybridize with HPV MM7; and SEQ ID NO:420-SEQ ID NO:429 and sequences fully complementary thereto, which hybridize with HPV MM8.

The foregoing and other features and advantages of the present invention will be more clearly understood through the following descriptions with reference to the drawings, wherein:

### BRIEF DESCRIPTION OF THE DRAWINGS

FIG. 1 is a schematic view showing the detector according to a preferred embodiment of the present invention;

FIG. 2(a) is a schematic view showing the detector according to a preferred embodiment of the present invention;

FIG. 2(b) is a schematic view illustrating the subtype of human papilloma viruses identified by each dot shown in FIG. 2(a);

FIG. 3(a) is the electrophoresis result showing the analyzed PCR products using primer set MY09/MY11 according to a preferred embodiment of the present invention;

FIG. 3(b) is the electrophoresis result showing the analyzed PCR products using primer set MY11/GP6+ according to a preferred embodiment of the present invention;

FIG. 3(c) is the electrophoresis result showing the analyzed PCR products using GAPDH primer set according to a preferred embodiment of the present invention;

FIG. 4(a) is the detecting result on the detector of detecting the PCR products using primer set MY09/MY11 of HPV positive clones according to a preferred embodiment of the present invention;

FIG. 4(b) is detecting result on the detector of detecting the PCR products using primer set MY11/GP6+ of HPV positive clones according to a preferred embodiment of the present invention;

FIG. 5 is a view showing the detecting result on the detectors of detecting samples according to a preferred embodiment of the present invention;

FIG. 6(a) is a schematic view showing the detector according to another preferred embodiment of the present invention;

FIG. 6(b) is a schematic view illustrating the subtype of human papilloma viruses identified by each dot shown in FIG. 6(a);

FIG. 7(a) is a view showing the detector stained with SYBR Green II according to a embodiment of the present invention; and

FIG. 7(b) is a view showing the detecting result on the detectors of detecting samples according to a preferred embodiment of the present invention.

### DETAILED DESCRIPTION OF THE PREFERRED EMBODIMENT

The present invention will now described more specifically with reference to the following embodiments. Papilloma viruses are small (50-60 nm), nonenveloped, and icosahedral DNA viruses. The DNA of many papilloma viruses, including over 50 human viruses, has been cloned and sequenced. Although there is a high degree of sequence divergence between species, all papilloma viruses share some common features of genome organization. The open reading frames (ORFs) of the virus genomes are designated an early region, a late region, and a long control region (LCR) of transcription. The early region contains genes E1-E8 (not all are present in all species), the late region contains genes L1 and L2 (where “E” denotes early and “L” denotes late), and the long control region (LCR) of transcription includes the promoter and enhancer for the viral early genes and the origin of replication. The early region encodes genes required for viral DNA replication, cellular proliferation, and, in some viruses, cellular transformation. The late region (about 3 kb) codes for the capsid proteins. L1 is the major capsid protein and is relatively well conserved among all the papilloma virus types. The L1 protein is about 500 amino acids in size. L1 probably induces the major humoral and cell-mediated responses to viral infection. The L2 proteins are about 500 amino acids in size, account for only a small proportion of the virion mass, and their function is not yet clear. The LCR region contains an origin of replication with binding sites for E1 and E2 and other cis acting sequences in the promoter and enhancer region.

Generally, PCR has been considered to be the most sensitive method for identifying HPV subtypes in biological samples. A number of different primer combinations amplifying DNA fragment from various regions of the HPV genome have been developed and used for the detection of HPV. However, primers amplifying DNA fragments in the conserved L1 region have become the most widely used in the clinical and epidemiological studies. It is because that certain region of the L1 gene presents a high degree of sequence variability in different HPV subtypes. In other words, the sequence variability among each HPV subtype could be the specific site for identifying each different HPV subtype.

In order to identify the various HPV subtypes, the Applicant focuses on the loci near the end of L1 gene to search the specific sequence variability as mentioned above. More specifically, the PCR fragment synthesized by the primer sets MY11/MY09 (as disclosed in Weimin et al., 1997, J. Clin. Microbiol. 35(6): 1304-1310) in the L1 region is the particular loci ranges where the Applicant refers to find the specific sequence variability for each HPV subtype in the present invention. Since the specific sequence variability for each HPV subtype is not only specific to a particular HPV subtype, but also distinguished from any other HPV subtype, consequently, the probes specifically hybridization with a particular HPV subtype could be selected for identifying or diagnosing HPV subtypes, which is also one of the main purposes of the present invention.

The PCR fragments synthesized by the primer sets MY11/MY09 in the L1 region are about 450 bp in length and had been published. The sequences of the fragments for each HPV subtype described in the invention are publicly available, for example, from the National Center for Biotechnology Information (NCBI) (e.g., www.ncbi.nih.gov). The 39 HPV subtypes identified in the invention includes HPV 6, HPV 11, HPV 16, HPV 18, HPV 26, HPV 31, HPV 32, HPV 33, HPV 35, HPV 37, HPV 39, HPV 42, HPV 43, HPV 44, HPV 45, HPV 51, HPV 52, HPV 53, HPV 54, HPV 55, HPV 56, HPV 58, HPV 59, HPV 61, HPV 62, HPV 66, HPV 67, HPV 68, HPV 69, HPV 70, HPV 72, HPV 74, HPV 82, HPV CP8061, HPV CP8034, HPV L1AE5, HPV MM4, HPV MM7 and HPV MM8. The original NCBI Accession number and the loci of the PCR fragments synthesized by the primer sets MY11/MY09 for different HPV subtypes are listed in Table 1:

TABLE 1

| | Accession | | |
|---|---|---|---|
| HPV subtype | number/length(bp) | loci/length(bp) | SEQ ID NO. |
| HPV 6 | NC_000904/8012 | 6743-7151/409 | 430 |
| HPV 11 | NC_001525/7931 | 6727-7135/409 | 431 |
| HPV 16 | NC_001526/7904 | 6602-7013/412 | 432 |
| HPV 18 | NC_001357/7857 | 6578-6992/415 | 433 |
| HPV 26 | NC_001583/7855 | 6553-6967/415 | 434 |
| HPV 31 | NC_001527/7912 | 6520-6931/412 | 435 |
| HPV 32 | NC_001586/7961 | 6837-7245/409 | 436 |
| HPV 33 | NC_001528/7909 | 6559-6967/409 | 437 |
| HPV 35 | NC_001529/7851 | 6542-6953/412 | 438 |
| HPV 37 | NC_001687/7421 | 6711-7125/415 | 439 |
| HPV 39 | NC_001535/7833 | 6605-7019/415 | 440 |
| HPV 42 | NC_001534/7917 | 6802-7210/409 | 441 |
| HPV 43 | U12504/455 | 21-435/415 | 442 |
| HPV 44 | NC_001689/7833 | 6647-7061/415 | 443 |
| HPV 45 | NC_001590/7858 | 6582-6996/415 | 444 |
| HPV 51 | NC_001533/7808 | 6486-6897/412 | 445 |
| HPV 52 | NC_001592/7942 | 6623-7031/409 | 446 |
| HPV 53 | NC_001593/7856 | 6614-7022/409 | 447 |
| HPV 54 | NC_001676/7759 | 6561-6972/412 | 448 |
| HPV 55 | NC_001692/7822 | 6647-7061/415 | 449 |
| HPV 56 | NC_001594/7844 | 6559-6967/409 | 450 |
| HPV 58 | NC_001443/7824 | 6608-7016/409 | 451 |
| HPV 59 | NC_001635/7896 | 6571-6985/415 | 452 |
| HPV 61 | NC_001694/7989 | 6732-7146/415 | 453 |
| HPV 62 | U12499/449 | 21-429/409 | 454 |
| HPV 66 | NC_001695/7824 | 6609-7017/409 | 455 |
| HPV 67 | D21208/7801 | 6584-6992/409 | 456 |
| HPV 68 | M73258/6042 | 2582-2996/415 | 457 |
| HPV 69 | NC 002171/7700 | 6509-6923/415 | 458 |
| HPV 70 | NC 001711/7905 | 6549-6963/415 | 459 |
| HPV 72 | X94164/7988 | 6758-7172/415 | 460 |
| HPV 74 | U40822/3891 | 1613-2027/415 | 461 |
| HPV 82 | AB027021/7871 | 6536-6950/415 | 462 |
| HPV CP8061 | U12479/452 | 21-432/412 | 463 |
| HPV CP8304 | U12480/452 | 21-432/412 | 464 |
| HPV L1AE5 | AF039910/364 | 11-360/350 | 465 |
| HPV MM4 | U12488/455 | 21-435/415 | 466 |
| HPV MM7 | U12489/452 | 21-432/412 | 467 |
| HPV MM8 | U12490/452 | 21-432/412 | 468 |

The sequences of the fragments of each HPV subtype described in the invention are listed below:

Human Papilloma Virus subtype 6 (6743-7151/409 bp)

Human Papilloma Virus subtype 6 (6743-7151/409 bp)

SEQ ID NO 430

| | |
|---|---|
| tatttgttgg ggtaatcaac tgtttgttac tgtggtagat | 60 |
| accacacgca gtaccaacat | |
| gacattatgt gcatccgtaa ctacatcttc cacatacacc | 120 |
| aattctgatt ataaagagta | |
| catgcgtcat gtggaagagt atgatttaca atttattttt | 180 |
| caattatgta gcattacatt | |
| gtctgctgaa gtaatggcct atattcacac aatgaatccc | 240 |
| tctgttttgg aagactggaa | |
| ctttgggtta tcgcctcccc caaatggtac attagaagat | 300 |
| acctataggt atgtgcagtc | |
| acaggccatt acctgtcaaa agcccactcc tgaaaaggaa | 360 |
| aagccagatc cctataagaa | |
| ccttagtttt tgggaggtta atttaaaaga aaagttttct | 409 |
| agtgaattg | |

Human Papilloma Virus subtype 11 (6727-7135/409
bp)

SEQ ID NO 431

| | |
|---|---|
| tatttgctgg ggaaaccact tgtttgttac tgtggtagat | 60 |
| accacacgca gtacaaatat | |
| gacactatgt gcatctgtgt ctaaatctgc tacatacact | 120 |
| aattcagatt ataaggaata | |
| catgcgccat gtggaggagt ttgatttaca gtttattttt | 180 |
| caattgtgta gcattacatt | |
| atctgcagaa gtcatggcct atatacacac aatgaatcct | 240 |
| tctgttttgg aggactggaa | |
| ctttggttta tcgcctccac caaatggtac actggaggat | 300 |
| acttatagat atgtacagtc | |
| acaggccatt acctgtcaga aacccacacc tgaaaaagaa | 360 |
| aaacaggatc cctataagga | |
| tatgagtttt tgggaggtta acttaaaaga aaagttttca | 409 |
| agtgaatta | |

Human Papilloma Virus subtype 16 (6602-7013/412
bp)

SEQ ID NO 432

| | |
|---|---|
| catttgttgg ggtaaccaac tatttgttac tgttgttgat | 60 |
| actacacgca gtacaaatat | |
| gtcattatgt gctgccatat ctacttcaga aactacatat | 120 |
| aaaaatacta actttaagga | |
| gtacctacga catggggagg aatatgattt acagtttatt | 180 |
| tttcaactgt gcaaaataac | |
| cttaactgca gacgttatga catacataca ttctatgaat | 240 |
| tccactattt tggaggactg | |
| gaattttggt ctacaacctc ccccaggagg cacactagaa | 300 |
| gatacttata ggtttgtaac | |
| ccaggcaatt gcttgtcaaa aacatacacc tccagcacct | 360 |
| aaagaagatg atccccttaa | |
| aaaatacact ttttgggaag taaatttaaa ggaaaagttt | 412 |
| tctgcagacc ta | |

Human Papilloma Virus subtype 18 (6587-6992/415
bp)

SEQ ID NO 433

| | |
|---|---|
| tgtttgctgg cataatcaat tatttgttac tgtggtagat | 60 |
| accactccca gtaccaattt | |
| aacaatatgt gcttctacac agtctcctgt acctgggcaa | 120 |
| tatgatgcta ccaaatttaa | |
| gcagtatagc agacatgttg aggaatatga tttgcagttt | 180 |
| atttttcagt tgtgtactat | |
| tactttaact gcagatgtta tgtcctatat tcatagtatg | 240 |
| aatagcagta ttttagagga | |
| ttggaacttt ggtgttcccc cccccccaac tactagtttg | 300 |
| gtggatacat atcgttttgt | |
| acaatctgtt gctattacct gtcaaaagga tgctgcaccg | 360 |
| gctgaaaata aggatcccta | |
| tgataagtta aagttttgga atgtggattt aaaggaaaag | 415 |
| ttttctttag actta | |

Human Papilloma Virus subtype 26 (6553-6967/415
bp)

SEQ ID NO 434

| | |
|---|---|
| tatctgttgg ggcaatcaat tgtttgttac ctgtgttgat | 60 |
| accacccgca gtactaacct | |
| taccattagt acattatctg cagcatctgc atccactcca | 120 |
| tttaaaccat ctgattataa | |
| acaatttata agacatggcg aagaatatga attacaattt | 180 |
| atatttcagt tgtgtaaaat | |
| aacacttaca acagatgtta tggcttacat acatttaatg | 240 |
| aatgcctcca tattggagga | |
| ttggaatttt ggactaacct tacctcccac tgctagtttg | 300 |
| gaagatgcct ataggtttat | |
| taaaaactct gctactacct gtcagcgtaa cgcccctcct | 360 |
| gtgccaaagg aagatccttt | |
| tcaaaaattt aaattttggg atgtagattt aaaagaaaaa | 415 |
| ttttctattg atttg | |

Human Papilloma Virus subtype 31 (6520-6931/412
bp)

SEQ ID NO 435

| | |
|---|---|
| tatttgttgg ggcaatcagt tatttgttac tgtggtagat | 60 |
| accacacgta gtaccaatat | |
| gtctgtttgt gctgcaattg caaacagtga tactacattt | 120 |
| aaaagtagta attttaaaga | |
| gtatttaaga catggtgagg aatttgattt acaatttata | 180 |
| tttcagttat gcaaaataac | |
| attatctgca gacataatga catatattca cagtatgaat | 240 |
| cctgctattt tggaagattg | |
| gaattttgga ttgaccacac ctccctcagg ttctttggag | 300 |
| gatacctata ggtttgtcac | |
| ctcacaggcc attacatgtc aaaaaactgc cccccaaaag | 360 |
| cccaaggaag atccatttaa | |
| agattatgta ttttgggagg ttaatttaaa agaaaagttt | 412 |
| tctgcagatt ta | |

Human Papilloma Virus subtype 32 (6837-7245/409
bp)

SEQ ID NO 436

| | |
|---|---|
| tatatgttgg ggtaatcaag tgtttctaac tgttgtggat | 60 |
| actacccgta gtactaacat | |
| gactgtgtgt gctactgtaa caactgaaga cacatacaag | 120 |
| tctactaact ttaaggaata | |
| tctacgccat gcagaggaat atgatataca gtttatattt | 180 |
| caattgtgca aaattacatt | |
| atctgtagag gttatgtcat atatccacac catgaatcct | 240 |
| gacatactag acgattggaa | |
| tgttggtgta gctccaccgc cctctggtac tttagaagat | 300 |
| agttatagat ttgtgcagtc | |
| tcaggccata cgatgtcaag ctaaggtaac agcacctgaa | 360 |
| aaaaaggatc ctttttctga | |
| ctattcattt tgggaagtaa atttatctga aaagttttct | 409 |
| agtgattta | |

Human Papilloma Virus subtype 33 (6559-6967/409
bp)

SEQ ID NO 437

| | |
|---|---|
| tatttgttgg ggcaatcagg tatttgttac tgtggtagat | 60 |
| accactcgca gtactaatat | |
| gactttatgc acacaagtaa ctagtgacag tacatataaa | 120 |
| aatgaaaatt ttaaagaata | |
| tataagacat gttgaagaat atgatctaca gtttgttttt | 180 |
| caactatgca aagttacctt | |
| aactgcagaa gttatgacat atattcatgc tatgaatcca | 240 |
| gatattttag aagattggca | |
| atttggttta acacctcctc catctgctag tttacaggat | 300 |
| acctataggt ttgttacctc | |
| tcaggctatt acgtgtcaaa aaacagtacc tccaaaggaa | 360 |
| aaggaagacc ccttaggtaa | |
| atatacattt tgggaagtgg atttaaagga aaaattttca | 409 |
| gcagattta | |

Human Papilloma Virus subtype 35 (6542-6953/412
bp)

SEQ ID NO 438

| | |
|---|---|
| tatttgttgg agtaaccaat tgtttgttac tgtagttgat | 60 |
| acaacccgta gtacaaatat | |
| gtctgtgtgt tctgctgtgt cttctagtga cagtacatat | 120 |
| aaaaatgaca attttaagga | |
| atatttaagg catggtgaag aatatgattt acagtttatt | 180 |
| tttcagttat gtaaaataac | |
| actaacagca gatgttatga catatattca tagtatgaac | 240 |
| ccgtccattt tagaggattg | |
| gaattttggc cttacaccac cgccttctgg taccttagag | 300 |
| gacacatatc gctatgtaac | |
| atcacaggct gtaacttgtc aaaaacccag tgcaccaaaa | 360 |
| cctaaagatg atccattaaa | |
| aaattatact ttttgggagg ttgatttaaa ggaaaagttt | 412 |
| tctgcagact ta | |

Human Papilloma Virus subtype 37 (6711-7125/415
bp)

SEQ ID NO 439

| | |
|---|---|
| cattttatgg ggtaatcaaa tgtttatcac agttgctgat | 60 |
| aatacacgga acacaaactt | |
| ttctattagt gtgtctactg acaatggcga agttacagaa | 120 |
| tataattctc aaacactcag | |
| agaataccta agacatgttg aagaatacca gctttcaatt | 180 |
| attttacaac tttgtaaagt | |
| tcctttaaag gctgaggttt taactcagat aaatgcaatg | 240 |
| aattctggta tattggaaga | |
| gtggcaatta ggatttgtac ctactccaga taattcagta | 300 |
| catgaccttt ataggtacat | |
| taattcaaag gctaccaagt gtcctgatgc agttgttgaa | 360 |
| aaagaaaagg aagatccctt | |
| tgcaaaatat acattttgga atgtagattt aactgaaaaa | 415 |
| ttatcattgg attta | |

Human Papilloma Virus subtype 39 (6605-7017/415
bp)

SEQ ID NO 440

| | |
|---|---|
| tatatgttgg cataatcaat tatttcttac tgttgtggac | 60 |
| actacccgta gtaccaactt | |
| tacattatct acctctatag agtcttccat accttctaca | 120 |
| tatgatcctt ctaagtttaa | |
| ggaatatacc aggcacgtgg aggagtatga tttacaattt | 180 |
| atatttcaac tgtgtactgt | |
| cacattaaca actgatgtta tgtcttatat tcacactatg | 240 |
| aattcctcta tattggacaa | |
| ttggaatttt gctgtagctc ctccaccatc tgccagtttg | 300 |
| gtagacactt acagatacct | |
| acagtctgca gccattacat gtcaaaagga tgctccagca | 360 |
| cctgaaaaga aagatccata | |
| tgacggtcta aagttttgga atgttgactt aagggaaaag | 415 |
| tttagtttgg aactt | |

Human Papilloma Virus subtype 42 (6802-7210/409
bp)

SEQ ID NO 441

| | |
|---|---|
| tatatgttgg ggaaatcagc tatttttaac tgtggttgat | 60 |
| actacccgta gtactaacat | |
| gactttgtgt gccactgcaa catctggtga tacatataca | 120 |
| gctgctaatt ttaaggaata | |
| tttaagacat gctgaagaat atgatgtgca atttatattt | 180 |
| caattgtgta aaataacatt | |
| aactgttgaa gttatgtcat atatacacaa tatgaatcct | 240 |
| aacatattag aggagtggaa | |
| tgttggtgtt gcaccaccac cttcaggaac tttagaagat | 300 |
| agttataggt atgtacaatc | |
| agaagctatt cgctgtcagg ctaaggtaac aacgccagaa | 360 |
| aaaaaggatc cttattcaga | |
| cttttggttt tgggaggtaa atttatctga aaagttttct | 409 |
| actgattta | |

Human Papilloma Virus subtype 43 (21-435/415 bp)

SEQ ID NO 442

| | |
|---|---|
| catttgtttt gggaatcagt tgtttgttac agtggtagat | 60 |
| accactcgta gtacaaactt | |
| gacgttatgt gcctctactg accctactgt gcccagtaca | 120 |
| tatgacaatg caaagtttaa | |
| ggaatacttg cggcatgtgg aagaatatga tctgcagttt | 180 |
| atatttcaat tatgcataat | |
| aacgctaaac ccagaggtta tgacatatat tcatactatg | 240 |
| gatcccacat tattagagga | |
| ctggaatttt ggtgtgtccc cacctgcctc tgcttctttg | 300 |
| gaagatactt atcgcttttt | |
| gtctaacaag gccattgcat gtcaaaaaaa tgctccccca | 360 |
| aaggaacggg aggatcccta | |
| taaaaagtat acattttggg atataaatct tacagaaaag | 415 |
| ttttctgcac aactt | |

Human Papilloma Virus subtype 44 (6647-7061/415
bp)

SEQ ID NO 443

| | |
|---|---|
| tatttgttgg ggaaatcagt tatttgttac tgttgtagat | 60 |
| actacccgta gtacaaacat | |
| gacaatatgt gctgccacta cacagtcccc tccgtctaca | 120 |
| tatactagtg aacaatataa | |
| gcaatacatg cgacatgttg aggagtttga cttacaattt | 180 |
| atgtttcaat tatgtagtat | |
| taccttaacg gcggaggtaa tggcctatct tcatactatg | 240 |
| aatgctggta ttttagaaca | |
| gtggaacttt gggttgtcgc cgcccccaaa tggtacctta | 300 |
| gaggacaaat acagatatgt | |
| gcagtcccag gocattacat gtcaaaagcc accccctgaa | 360 |
| aaggcaaagc aggaccccta | |
| tgcaaaatta agtttttggg aggtggatct tagagaaaag | 415 |
| ttttctagtg agttg | |

Human Papilloma Virus subtype 45 (6582-6996/415
bp)

SEQ ID NO 444

| | |
|---|---|
| tatttgttgg cataatcagt tgtttgttac tgtagtggac | 60 |
| actacccgca gtactaattt | |
| aacattatgt gcctctacac aaaatcctgt gccaagtaca | 120 |
| tatgacccta ctaagtttaa | |
| gcagtatagt agacatgtgg aggaatatga tttacagttt | 180 |
| atttttcagt tgtgcactat | |
| tactttaact gcagaggtta tgtcatatat ccatagtatg | 240 |
| aatagtagta tattagaaaa | |
| ttggaatttt ggtgtccctc caccacctac tacaagtttg | 300 |
| gtggatacat atcgttttgt | |
| gcaatcagtt gctgttacct gtcaaaagga tactacacct | 360 |
| ccagaaaagc aggatccata | |
| tgataaatta aagttttgga ctgttgacct aaaggaaaaa | 415 |
| ttttcctccg atttg | |

Human Papilloma Virus subtype 51 (6486-6897/412
bp)

SEQ ID NO 445

| | |
|---|---|
| catttgctgg aacaatcagc tttttattac ctgtgttgat | 60 |
| actaccagaa gtacaaattt | |
| aactattagc actgccactg ctgcggtttc cccaacattt | 120 |
| actccaagta actttaagca | |
| atatattagg catggggaag agtatgaatt gcaatttatt | 180 |
| tttcaattat gtaaaattac | |
| tttaactaca gaggtaatgg cttatttaca cacaatggat | 240 |
| cctaccattc ttgaacagtg | |
| gaattttgga ttaacattac ctccgtctgc tagtttggag | 300 |
| gatgcatata ggtttgttag | |
| aaatgcagct actagctgtc aaaaggacac ccctccacag | 360 |
| gctaagccag atcctttggc | |
| caaatataaa ttttgggatg ttgatttaaa ggaacgattt | 412 |
| tctttagatt ta | |

Human Papilloma Virus subtype 52 (6623-7031/409
bp)

SEQ ID NO 446

| | |
|---|---|
| catatgttgg ggcaatcagt tgtttgtcac agttgtggat | 60 |
| accactcgta gcactaacat | |
| gactttatgt gctgaggtta aaaaggaaag cacatataaa | 120 |
| aatgaaaatt ttaaggaata | |
| ccttcgtcat ggcgaggaat ttgatttaca atttattttt | 180 |
| caattgtgca aaattacatt | |
| aacagctgat gttatgacat acattcataa gatggatgcc | 240 |
| actattttag aggactggca | |
| atttggcctt accccaccac cgtctgcatc tttggaggac | 300 |
| acatacagat ttgtcacttc | |
| tactgctata acttgtcaaa aaaacacacc acctaaagga | 360 |
| aaggaagatc ctttaaagga | |
| ctatatgttt tgggaggtgg atttaaaaga aaagttttct | 409 |
| gcagattta | |

Human Papilloma Virus subtype 53 (6614-7022/409
bp)

SEQ ID NO 447

| | |
|---|---|
| catctgttgg aacaatcagt tatttgtaac tgttgtggat | 60 |
| accaccagga atacaaacat | |
| gactctttcc gcaaccacac agtctatgtc tacatataat | 120 |
| tcaaagcaaa ttaaacagta | |
| tgttagacat gcagaggaat atgaattaca atttgtgttt | 180 |
| caactatgta aaatatccct | |
| gtctgctgag gttatggcct atttacatac tatgaattct | 240 |
| accttactgg aagactggaa | |
| tataggtttg tcgcctcctg ttgccactag cttagaggac | 300 |
| aaatacagat atgtgaaaag | |
| tgcagctata acctgtcaaa aggatcagcc ccctcctgaa | 360 |
| aagcaggacc cactatctaa | |
| atataaattt tgggaggtca atttgcaaaa cagtttttct | 409 |
| gctgatttg | |

Human Papilloma Virus subtype 54 (6561-6972/412
bp)

SEQ ID NO 448

| | |
|---|---|
| tatttgttgg ggcaatcagg tgtttttaac agttgtagat | 60 |
| accacccgta gtactaacct | |
| aacattgtgt gctacagcat ccacgcagga tagctttaat | 120 |
| aattctgact ttagggagta | |
| tattagacat gtggaggaat atgatttaca gtttatattt | 180 |
| cagttatgta ccataaccct | |
| tacagoagat gttatggcct atattcatgg aatgaatccc | 240 |
| actattctag aggactggaa | |
| ctttggtata acccccccag ctacaagtag tttggaggac | 300 |
| acatataggt ttgtacagtc | |
| acaggccatt gcatgtcaaa agaataatgc ccctgcaaag | 360 |
| gaaaaggagg atccttacag | |
| taaatttaat ttttggactg ttgaccttaa ggaacgattt | 412 |
| tcatctgacc tt | |

Human Papilloma Virus subtype 55 (6647-7061/415
bp)

SEQ ID NO 449

| | |
|---|---|
| tatttgttgg gggaatcagt tatttgttac tgttgtagat | 60 |
| actacacgta gtacaaacat | |
| gacaatatgt gctgctacaa ctcagtctcc atctacaaca | 120 |
| tataatagta cagaatataa | |
| acaatacatg cgacatgttg aggagtttga cttacagttt | 180 |
| atgtttcaat tatgtagtat | |
| taccttaact gctgaggtaa tggcctattt acataccatg | 240 |
| aatcctggta ttttggaaca | |
| gtggaacttt gggttgtcgc cacccccaaa tggtacctta | 300 |
| gaagacaaat acagatatgt | |
| gcagtcacag gocattacat gtcaaaagcc tccccctgaa | 360 |
| aaggcaaagc aggaccccta | |
| tgcaaaatta agtttttggg aggtagatct cagagaaaag | 415 |
| ttttctagtg agtta | |

Human Papilloma Virus subtype 56 (6559-6967/409
bp)

SEQ ID NO 450

| | |
|---|---|
| catttgctgg ggtaatcaat tatttgttac tgtagtagat | 60 |
| actactagaa gtactaacat | |
| gactattagt actgctacag aacagttaag taaatatgat | 120 |
| gcacgaaaaa ttaatcagta | |
| ccttagacat gtggaggaat atgaattaca atttgttttt | 180 |
| caattatgca aaattacttt | |
| gtctgcagag gttatggcat atttacataa tatgaatgct | 240 |
| aacctactgg aggactggaa | |
| tattgggtta tccccgccag tggccaccag cctagaagat | 300 |
| aaatatagat atgttagaag | |
| cacagctata acatgtcaac gggaacagcc accaacagaa | 360 |
| aaacaggacc cattagctaa | |
| atataaattt tgggatgtta acttacagga cagtttttct | 419 |
| acagacctg | |

Human Papilloma Virus subtype 58 (6608-7016/409
bp)

SEQ ID NO 451

| | |
|---|---|
| catttgctgg ggcaatcagt tatttgttac cgtggttgat | 60 |
| accactcgta gcactaatat | |
| gacattatgc actgaagtaa ctaaggaagg tacatataaa | 120 |
| aatgataatt ttaaggaata | |
| tgtacgtcat gttgaagaat atgacttaca gtttgttttt | 180 |
| cagctttgca aaattacact | |
| aactgcagag ataatgacat atatacatac tatggattcc | 240 |
| aatattttgg aggactggca | |
| atttggttta acacctcctc cgtctgccag tttacaggac | 300 |
| acatatagat ttgttacctc | |
| ccaggctatt acttgccaaa aaacagcacc ccctaaagaa | 360 |
| aaggaagatc cattaaataa | |
| atatactttt tgggaggtta acttaaagga aaagttttct | 409 |
| gcagatcta | |

Human Papilloma Virus subtype 59 (6571-6985/415
bp)

SEQ ID NO 452

| | |
|---|---|
| tatatgttgg cacaatcaat tgtttttaac agttgtagat | 60 |
| actactcgca gcaccaatct | |
| ttctgtgtgt gcttctacta cttcttctat tcctaatgta | 120 |
| tacacaccta ccagttttaa | |
| agaatatgcc agacatgtgg aggaatttga tttgcagttt | 180 |
| atatttcaac tgtgtaaaat | |
| aacattaact acagaggtaa tgtcatacat tcataatatg | 240 |
| aataccacta ttttggagga | |
| ttggaatttt ggtgttacac cacctcctac tgctagttta | 300 |
| gttgacacat accgttttgt | |
| tcaatctgct gctgtaactt gtcaaaagga caccgcaccg | 360 |
| ccagttaaac aggaccctta | |
| tgacaaacta aagttttggc ctgtagatct taaggaaagg | 415 |
| ttttctgcag atctt | |

Human Papilloma Virus subtype 61 (6732-7146/415
bp)

SEQ ID NO 453

| | |
|---|---|
| tatttgttgg tttaatgaat tgtttgtaac cgttgtggat | 60 |
| accacccgca gtactaattt | |
| aaccatttgt actgctacat ccccccctgt atctgaatat | 120 |
| aaagccacaa gctttaggga | |
| atatttgcgc catacagagg agtttgattt gcaatttatt | 180 |
| tttcagttat gtaaaataca | |
| tttaacccct gaaattatgg cctacctaca taatatgaat | 240 |
| aaggccttgt tggatgactg | |
| gaactttggt gtggtaccac caccctctac cagtttagaa | 300 |
| gacacatata ggtttttgca | |
| gtccagagct attacatgtc agaagggtgc tgctgccccg | 360 |
| ccgcccaagg aggatcgcta | |
| tgccaagtta tccttttgga ctgttgattt acgagacaag | 415 |
| ttttccactg atttg | |

Human Papilloma Virus subtype 62 (21-429/409 bp)

SEQ ID NO 454

| | |
|---|---|
| tatttgttgg tttaatgaac tgtttgttac tgtggtggat | 60 |
| actaccagaa gtactaattt | |
| tactatttgt accgcctcca ctgctgcagc agaatacacg | 120 |
| gctaccaact ttagggaatt | |
| tttgcgacac acggaggaat ttgatttgca atttatattt | 180 |
| caattgtgca aaatacagtt | |
| aacccccgaa attatggcct acctgcataa tatgaacaag | 240 |
| gaccttttgg atgactggaa | |
| ctttggggtt ttacctcccc cttccactag tttagatgag | 300 |
| acatatcact atttcgagtc | |
| tcgggctatt acatgtcaaa gggggctgcc tacccgtccc | 360 |
| aaggtggacc cgtatgcgca | |
| aatgacattt tggactgtgg atcttaagga caagttgtct | 409 |
| actgatttg | |

Human Papilloma Virus subtype 66 (6609-7017/409
bp)

SEQ ID NO 455

| | |
|---|---|
| catatgctgg ggtaatcagg tatttgttac tgttgtggat | 60 |
| actaccagaa gcaccaacat | |
| gactattaat gcagctaaaa gcacattaac taaatatgat | 120 |
| gcccgtgaaa tcaatcaata | |
| ccttcgccat gtggaggaat atgaactaca gtttgtgttt | 180 |
| caactttgta aaataacctt | |
| aactgcagaa gttatggcat atttgcataa tatgaataat | 240 |
| actttattag acgattggaa | |
| tattggctta tccccaccag ttgcaactag cttagaggat | 300 |
| aaatataggt atattaaaag | |
| cacagctatt acatgtcaga gggaacagcc ccctgcagaa | 360 |
| aagcaggatc ccctggctaa | |
| atataagttt tgggaagtta atttacagga cagcttttct | 409 |
| gcagacctg | |

Human Papilloma Virus subtype 67 (6584-6992/409
bp)

SEQ ID NO 456

| | |
|---|---|
| tatatgctgg ggtaatcaaa tatttgttac tgttgtagac | 60 |
| actacacgta gtaccaacat | |
| gactttatgt tctgaggaaa aatcagaggc tacatacaaa | 120 |
| aatgaaaact ttaaggaata | |
| ccttagacat gtggaagaat atgatttgca gtttatattt | 180 |
| cagctgtgca aaatatccct | |
| tactgcaaat gttatgcaat acatacacac catgaatcca | 240 |
| gatatattag aggactggca | |
| atttggcctt acaccacctc cttcaggtaa tttacaggac | 300 |
| acatatagat ttgttacctc | |
| gcaggctatt acctgtcaaa aaacatcccc tccaacagca | 360 |
| aaggaagatc ctcttaaaaa | |
| gtacagtttt tgggaaatca atttaaagga aaaattttct | 409 |
| gcagattta | |

Human Papilloma Virus subtype 68 (2582-2996/415
bp)

SEQ ID NO 457

| | |
|---|---|
| tatttgttgg cataatcaat tatttcttac tgttgtggat | 60 |
| accactcgca gtaccaattt | |
| tactttgtct actactactg aatcagctgt accaaatatt | 120 |
| tatgatccta ataaatttaa | |
| ggaatatatt aggcatgttg aggaatatga tttgcaattt | 180 |
| atatttcagt tgtgtactat | |
| aacattgtcc actgatgtaa tgtcctatat acatactatg | 240 |
| aatcctgcta ttttggatga | |
| ttggaatttt ggtgttgccc ctccaccatc tgctagtctt | 300 |
| gtagatacat accgctatct | |
| gcaatcagca gcaattacat gtcaaaaaga cgcccctgca | 360 |
| cctactaaaa aggatccata | |
| tgatggctta aacttttgga atgtaaattt aaaggaaaag | 415 |
| tttagttctg aactg | |

Human Papilloma Virus subtype 69 (6509-6923/415
bp)

SEQ ID NO 458

| | |
|---|---|
| catttgttgg ggcaaccaat tgtttgttac ttgtgtagat | 60 |
| actacccgca gtaccaacct | |
| cactattagt actgtatctg cacaatctgc atctgccact | 120 |
| tttaaaccat cagattataa | |
| gcagtttata aggcatggtg aggaatatga attacagttt | 180 |
| atatttcaat tgtgtaaaat | |
| tactcttacc actgatgtaa tggcctatat ccatacaatg | 240 |
| aattctacta ttttggaaaa | |
| ttggaatttt ggccttacct tgcctcctac tgctagtttg | 300 |
| gaagatgcat ataggtttat | |
| taaaaattca gctactacat gtcaacgcga tgcccctgca | 360 |
| cagcccaagg aggatccatt | |
| tagtaaatta aaattttggg acgttgatct taaagaaaag | 415 |
| ttttctattg attta | |

Human Papilloma Virus subtype 70 (6549-6963/415
bp)

SEQ ID NO 459

| | |
|---|---|
| catttgttgg cataaccagt tgtttattac tgtggtggac | 60 |
| actacacgta gtactaattt | |
| tacattgtct gcctgcaccg aaacggccat acctgctgta | 120 |
| tatagcccta caaagtttaa | |
| ggaatatact aggcatgtgg aggaatatga tttacaattt | 180 |
| atatttcaat tgtgtactat | |
| cacattaact gctgacgtta tggcctacat ccatactatg | 240 |
| aatcctgcaa ttttggacaa | |
| ttggaatata ggagttaccc ctccaccatc tgcaagcttg | 300 |
| gtggacacgt ataggtattt | |
| acaatcagca gctatagcat gtcaaaagga tgctcctaca | 360 |
| cctgaaaaaa aggatcccta | |
| tgacgattta aaattttgga atgttgattt aaaggaaaag | 415 |
| tttagtacag aacta | |

Human Papilloma Virus subtype 72 (6758-7172/415
bp)

SEQ ID NO 460

| | |
|---|---|
| catctgttgg tttaatgagc tttttgtgac agttgtagat | 60 |
| actactcgca gtactaatgt | |
| aactatttgt actgccacag cgtcctctgt atcagaatat | 120 |
| acagcttcta attttcgtga | |
| gtatcttcgc cacactgagg aatttgattt gcagtttata | 180 |
| tttcaactgt gtaaaattca | |
| cttaactcct gaaattatgg cctacttgca caatatgaat | 240 |
| aaggccttat tggatgactg | |
| gaattttggt gtggtgcctc ctccttctac cagtttggat | 300 |
| gatacctata ggtttttgca | |
| gtctcgtgcc attacctgtc aaaagggggc tgccacccct | 360 |
| cctcctaaag aagatccata | |
| tgctaactta tccttttgga ctgtggattt aaaggacaaa | 415 |
| ttttccactg acttg | |

Human Papilloma Virus subtype 74 (1613-2027/415
bp)

SEQ ID NO 461

| | |
|---|---|
| tatttgttgg ggtaatcaat tatttgttac agttgtggat | 60 |
| accacacgca gtactaacat | |
| gactgtgtgt gctcctacct cacaatcgcc ttctgctaca | 120 |
| tataatagtt cagactacaa | |
| acaatacatg cgacatgtgg aggaatttga tttgcaattt | 180 |
| atttttcaat tatgtagtat | |
| taagttaact gctgaggtta tggcctatat tcatactatg | 240 |
| aatcctacag ttttagaaga | |
| gtggaacttt gggctaacgc ctccccccaa tggtacttta | 300 |
| gaagacacct acagatatgt | |
| gcagtcccag gctattacat gtcaaaaacc tacgcctgat | 360 |
| aaagcaaagc ccaatcccta | |
| tgcaaattta agtttttggg aagttaatct taaggaaaag | 415 |
| ttttctagtg aatta | |

Human Papilloma Virus subtype 82 (6536-6950/415
bp)

SEQ ID NO 462

| | |
|---|---|
| catttgctgg aataatcagc tttttattac ttgtgttgac | 60 |
| actactaaaa gtaccaattt | |
| aaccattagc actgctgtta ctccatctgt tgcacaaaca | 120 |
| tttactccag caaactttaa | |
| gcagtacatt aggcatgggg aagaatatga attgcaattt | 180 |
| atatttcaat tgtgtaaaat | |
| cactttaact actgaaatta tggcttacct gcacaccatg | 240 |
| gattctacaa ttttagaaca | |
| gtggaatttt ggattaacat tgcccccctc cgctagtttg | 300 |
| gaggatgcct atcgatttgt | |
| aaaaaatgca gcaacatcct gtcacaagga cagtcctcca | 360 |
| caggctaaag aagacccttt | |
| ggcaaaatat aaattttgga atgtagacct taaggaacgc | 415 |
| ttttctttgg atttg | |

Human Papilloma Virus subtype CP8061 (21-432/412
bp)

SEQ ID NO 463

| | |
|---|---|
| catttgttgg ggcaatcagc tttttgtaac agttgtggac | 60 |
| acatcacgta gtacaaatat | |
| gtccatctgt gctaccaaaa ctgttgagtc tacatataaa | 120 |
| gcctctagtt tcatggaata | |
| tttgagacat ggagaagaat ttgatttgca atttatattt | 180 |
| caactatgtg ttattaattt | |
| aacagctgaa attatggcct acttacatcg catggatgct | 240 |
| acattactgg aggactggaa | |
| tttttggttc ttaccacctc ctactgctag tcttggtgat | 300 |
| acctaccgct ttttacagtc | |
| tcaggccata acctgtcaga aaaacagtcc tcctcctgca | 360 |
| gaaaaaaagg acccctatgc | |
| agatcttaca ttttgggagg tggatttaaa ggagcggttt | 412 |
| tcactagaat tg | |

Human Papilloma Virus subtype CP8304 (21-432/412
bp)

SEQ ID NO 464

| | |
|---|---|
| tatttgttgg tttaatgaaa tgtttgttac agtggtggat | 60 |
| actaccagaa gcaccaattt | |
| tactatttgc acagctacat ctgctgctgc agaatacaag | 120 |
| gcctctaact ttaaggaatt | |
| tctgcgccat acagaggaat atgatttgca gtttattttc | 180 |
| caattatgta aaatacagtt | |
| aacaccagaa attatggcct acttacataa tatgaacaag | 240 |
| gcactgttgg atgattggaa | |
| ttttggtgtg ttgccacctc cttccaccag tttagatgac | 300 |
| acatatcgct ttttacagtc | |
| tcgggccatt acctgtcaaa agggtgctgc tgcccctgcg | 360 |
| cccaaagagg acccttatgc | |
| cgacatgtca ttttggacag ttgaccttaa ggacaagttg | 412 |
| tctactgatt tg | |

Human Papilloma Virus subtype L1AE5 (11-360/350
bp)

SEQ ID NO 465

| | |
|---|---|
| ggcacaacca attatttata actgtggtag acacaacacg | 60 |
| tagtaccaat cttaccttat | |
| ctactgcaac tactaatcca gttccatcta tatatgaacc | 120 |
| ttctaaattt aaggaataca | |
| cacgccatgt agaggaatat gatttacaat ttatatttca | 180 |
| attgtgtaaa attacactta | |
| ctactgatgt tatgtcttat atacataaca tggatcctac | 240 |
| tattttagat agttggaatt | |
| ttggtgttag tcctccccca tctgctagct tagtagatac | 300 |
| atataggttt ttacagtcat | |
| ctgccattac atgtcagaag gatgtggttg ttccacaaaa | 350 |
| aaaggatcca | |

Human Papilloma Virus subtype MM4 (21-435/415 bp)

SEQ ID NO 466

| | |
|---|---|
| catttgctgg aataatcagc tttttattac ttgtgttgac | 60 |
| actactagaa gtaccaattt | |
| aaccattagc actgctgtta ctcaatctgt tgcacaaaca | 120 |
| tttactccag caaactttaa | |
| gcaatacatt aggcatgggg aagaatatga attgcaattt | 180 |
| atatttcaat tgtgtaaaat | |
| cactttaact actgaaatta tggcttacct gcacaccatg | 240 |
| gattctacaa ttttagaaca | |
| gtggaatttt ggattaacct tgcccccctc agctagtttg | 300 |
| gaggatgcct atcgatttgt | |
| aaaaaatgca gcaacatcct gtcacaagga cagtcctcca | 360 |
| caggctaaac aagacccttt | |
| ggcaaaatat aaattttgga atgtagacct taaggaacgc | 415 |
| ttttctttgg atttg | |

Human Papilloma Virus subtype MM7 (21-432/412 bp)

SEQ ID NO 467

| | |
|---|---|
| catttgttgg tttaatgagt tatttgttac agttgtagat | 60 |
| actacccgca gtaccaatat | |
| tactatttca gctgctgcta cacaggctaa tgaatacaca | 120 |
| gcctctaact ttaaggaata | |
| cctccgccac accgaggaat atgacttaca ggttatattg | 180 |
| caactttgca aaatacatct | |
| tacccctgaa attatggcat acctacatag tatgaatgaa | 240 |
| catttattgg atgagtggaa | |
| ttttggcgtg ttaccacctc cttccaccag ccttgatgat | 300 |
| acctatcgct atctgcagtc | |
| ccgtgctatt acctgccaaa agggtccttc cgcccctgcc | 360 |
| cctaaaaagg atccttatga | |
| tggccttgta ttttgggagg ttgatttaaa ggacaaacta | 412 |
| tccacagatt tg | |

Human Papilloma Virus subtype MM8 (21-432/412 bp)

SEQ ID NO 468

| | |
|---|---|
| tatatgctgg tttaatcaat tgtttgtcac ggtggtggat | 60 |
| accacccgca gcaccaattt | |
| tactattagt gctgctacca acaccgaatc agaatataaa | 120 |
| cctaccaatt ttaaggaata | |
| cctaagacat gtggaggaat atgatttgca gtttatattc | 180 |
| cagttgtgta aggtccgtct | |
| gactccagag gtcatgtcct atttacatac tatgaatgac | 240 |
| tccttattag atgagtggaa | |
| ttttggtgtt gtgccccctc cctccacaag tttagatgat | 300 |
| acctataggt acttgcagtc | |
| tcgcgccatt acttgccaaa agggggccgc cgccgccaag | 360 |
| cctaaggaag atccttatgc | |
| tggcatgtcc ttttgggatg tagatttaaa ggacaagttt | 412 |
| tctactgatt tg | |

In order to find the specific probes for identifying or diagnosing HPV subtypes, some sequence analysis software are used for finding the variety sites among the above listed sequences of different HPV subtypes, e.g., DNASTAR. The above 450-bp sequences of 39 HPV subtypes are respectively divided into several fragments and analyzed by the software. Preferably, the genetic identify compared to other HPV subtypes must be lower than 30% for finding suitable probes with high specificity. After identifying the variety sites having low genetic identity in sequences of each HPV subtype, the probes for each HPV subtype are respectively designed to specifically hybridize with these variety sites. Then, the designed probes are tested for their specificities to the corresponding HPV subtypes respectively. Preferably, the probes are 15-30 base pairs in length. Ultimately, 9-12 probes with high specificity are found for each HPV subtype. The sequences of the probes for each- HPV subtype are listed below.

UZ,14/19 HPV 6

| SEQ ID | | |
|---|---|---|
| NO | 5′→3′ | Locus in HPV 6 |
| 1 | CATCCGTAACTACATCTTCC | 6814-6833 |
| 2 | ATCCGTAACTACATCTTCCA | 6815-6834 |
| 3 | CTACATCTTCCACATACACCAA | 6823-6844 |
| 4 | CATCTTCCACATACACCAAT | 6826-6845 |
| 5 | ATCTTCCACATACACCAATT | 6827-6846 |
| 6 | CCACATACACCAATTCTGAT | 6832-6851 |
| 7 | TAGCATTACATTGTCTGCTGAAG | 6911-6933 |
| 8 | TCCCTCTGTTTTGGAAGAC | 6959-6977 |
| 09 | GTTATCGCCTCCCCCAAATGGTACAT | 6989-7014 |
| 10 | CTATAGGTATGTGCAGTCACAG | 7025-7046 |
| 11 | GCCCACTCCTGAAAAGGAA | 7064-7082 |
| 12 | CTATAAGAACCTTAGT | 7094-7109 |

HPV 11

| SEQ ID | | |
|---|---|---|
| NO | 5′→3′ | Locus in HPV 11 |
| 13 | ATCTGTGTCTAAATC | 6799-6813 |
| 14 | TCTGTGTCTAAATCTGCTAC | 6800-6819 |
| 15 | ATCTGTGTCTAAATCTGCTACATACA | 6799-6824 |
| 16 | TGCATCTGTGTCTAAATCTG | 6796-6815 |
| 17 | AAATCTGCTACATACACTAA | 6809-6828 |
| 18 | CTAAATCTGCTACATACACTA | 6807-6827 |
| 19 | CTACATACACTAATTCAGAT | 6816-6835 |
| 20 | TAGCATTACATTATCTGCAGAAG | 6895-6917 |
| 21 | TCCTTCTGTTTTGGAGGAC | 6943-6961 |
| 22 | TTTATCGCCTCCACCAAATGGTACAC | 6973-6998 |
| 23 | TTATAGATATGTACAGTCACAGGCC | 7009-7033 |
| 24 | ACCCACACCTGAAAAAGAAAAAC | 7048-7070 |

HPV 16

| SEQ ID | | |
|---|---|---|
| NO | 5′→3′ | Locus in HPV 16 |
| 25 | TATGTCATTATGTGCTGCCA | 6659-6678 |
| 26 | GTGCTGCCATATCTACTTCA | 6670-6689 |
| 27 | TGCCATATCTACTTC | 6674-6688 |
| 28 | TATCTACTTCAGAAACTACA | 6679-6698 |
| 29 | CTACTTCAGAAACTACATATAA | 6682-6703 |
| 30 | ATAAAAATACTAACTTTAAG | 6700-6719 |
| 31 | CAAAATAACCTTAACTGCAGACG | 6773-6795 |
| 32 | TTCCACTATTTTGGAGGAC | 6821-6839 |
| 33 | TCTACAACCTCCCCCAGGAGGCACAC | 6851-6876 |
| 34 | TTATAGGTTTGTAACCCAG | 6887-6905 |
| 35 | ACATACACCTCCAGCACCT | 6923-6941 |
| 36 | CCTTAAAAAATACACT | 6956-6971 |

HPV 18

| SEQ ID | | |
|---|---|---|
| NO | 5′→3′ | Locus in HPV 18 |
| 37 | TTCTACACAGTCTCC | 6650-6664 |
| 38 | CAGTCTCCTGTACCTGGGCA | 6657-6676 |
| 39 | AGTCTCCTGTACCTGGGCAA | 6658-6677 |
| 40 | TCTCCTGTACCTGGGCAATATGA | 6660-6682 |
| 41 | CTGTACCTGGGCAATATGAT | 6664-6683 |
| 42 | ATGATGCTACCAAATTTAAG | 6679-6698 |
| 43 | TACTATTACTTTAACTGCAGATG | 6752-6774 |
| 44 | TAGCAGTATTTTAGAGGAT | 6800-6818 |
| 45 | TGTTCCCCCCCCCCCAACTACTAGTT | 6830-6855 |
| 46 | ATATCGTTTTGTACAATCTGTT | 6866-6887 |
| 47 | GGATGCTGCACCGGCTGAA | 6905-6923 |
| 48 | CTATGATAAGTTAAAG | 6935-6950 |

HPV 26

| SEQ ID | | |
|---|---|---|
| NO | 5′→3′ | Locus in HPV 26 |
| 49 | TAGTACATTATCTGCAGCAT | 6619-6638 |
| 50 | ATTATCTGCAGCATC | 6625-6639 |
| 51 | TGCAGCATCTGCATCCACTC | 6631-6650 |
| 52 | GCATCTGCATCCACTCCATTTAAA | 6635-6658 |
| 53 | CTCCATTTAAACCATCTGAT | 6648-6667 |
| 54 | TAAAATAACACTTACAACAGATG | 6727-6749 |
| 55 | TGCCTCCATATTGGAGGAT | 6775-6793 |
| 56 | ACTAACCTTACCTCCCACTGCTAGTT | 6805-6830 |
| 57 | CTATAGGTTTATTAAAAACTCT | 6841-6862 |
| 58 | TAACGCCCCTCCTGTGCCA | 6880-6898 |

HPV 31

| SEQ ID | | |
|---|---|---|
| NO | 5′→3′ | Locus in HPV 31 |
| 59 | TGCAATTGCAAACAG | 6592-6606 |
| 60 | GCAATTGCAAACAGTGATAC | 6593-6612 |
| 61 | CAATTGCAAACAGTGATACT | 6594-6613 |
| 62 | GCAAACAGTGATACTACATTTAA | 6599-6621 |
| 63 | CTACATTTAAAAGTAGTAAT | 6612-6631 |
| 64 | CAAAATAACATTATCTGCAGACA | 6691-6713 |
| 65 | TCCTGCTATTTTGGAAGAT | 6739-6757 |
| 66 | ATTGACCACACCTCCCTCAGGTTCTT | 6769-6794 |
| 67 | CTATAGGTTTGTCACCTCACAG | 6805-6826 |
| 68 | AACTGCCCCCCAAAAGCCC | 6844-6862 |

HPV 32

| SEQ ID | | |
|---|---|---|
| NO | 5′→3′ | Locus in HPV 32 |
| 69 | TGCTACTGTAACAACTGAAG | 6906-6925 |
| 70 | GCTACTGTAACAACTGAAGA | 6907-6926 |
| 71 | TACTGTAACAACTGA | 6909-6923 |
| 72 | ACTGTAACAACTGAAGACAC | 6910-6929 |
| 73 | CAACTGAAGACACATACAAGTC | 6917-6938 |
| 74 | CAAAATTACATTATCTGTAGAGG | 7005-7027 |
| 75 | TCCTGACATACTAGACGAT | 7053-7071 |
| 76 | TGTAGCTCCACCGCCCTCTGGTACTT | 7083-7108 |
| 77 | TTATAGATTTGTGCAGTCTCAG | 7119-7140 |
| 78 | TAAGGTAACAGCACCTGAA | 7158-7176 |
| 79 | TTTTTCTGACTATTCA | 7188-7203 |

HPV 33

| SEQ ID | | |
|---|---|---|
| NO | 5′→3′ | Locus in HPV 33 |
| 80 | TATGCACACAAGTAACTAGT | 6624-6643 |
| 81 | CACACAAGTAACTAG | 6628-6642 |
| 82 | ACAAGTAACTAGTGACAGTA | 6631-6650 |
| 83 | GTAACTAGTGACAGTACATATAA | 6635-6657 |
| 84 | GTACATATAAAAATGAAAAT | 6648-6667 |
| 85 | CAAAGTTACCTTAACTGCAGAAG | 6727-6749 |
| 86 | TCCAGATATTTTAGAAGAT | 6775-6793 |
| 87 | TTTAACACCTCCTCCATCTGCTAGTT | 6805-6830 |
| 88 | CTATAGGTTTGTTACCTCTCAG | 6841-6862 |
| 89 | AACAGTACCTCCAAAGGAA | 6880-6898 |
| 90 | CTTAGGTAAATATACA | 6910-6925 |

HPV 35

| SEQ ID | | |
|---|---|---|
| NO | 5′→3′ | Locus in HPV 35 |
| 91 | TCTGCTGTGTCTTCTAGTGA | 6612-6631 |
| 92 | TGCTGTGTCTTCTAG | 6614-6628 |
| 93 | GTGTCTTCTAGTGACAGTAC | 6618-6637 |
| 94 | CTTCTAGTGACAGTACATATAAA | 6622-6644 |
| 95 | GTACATATAAAAATGACAAT | 6634-6653 |
| 96 | TAAAATAACACTAACAGCAGATG | 6713-6735 |
| 97 | CCCGTCCATTTTAGAGGAT | 6761-6779 |
| 98 | CCTTACACCACCGCCTTCTGGTACCT | 6791-6816 |
| 99 | ATATCGCTATGTAACATCACAG | 6827-6848 |
| 100 | ACCCAGTGCACCAAAACCT | 6866-6884 |

HPV 37

| SEQ ID | | |
|---|---|---|
| NO | 5′→3′ | Locus in HPV 37 |
| 101 | TGTCTACTGACAATG | 6782-6796 |
| 102 | TGTCTACTGACAATGGCGAA | 6782-6801 |
| 103 | TGACAATGGCGAAGTTACAG | 6789-6808 |
| 104 | GACAATGGCGAAGTTACAGA | 6790-6809 |
| 105 | AATGGCGAAGTTACAGAATA | 6793-6812 |
| 106 | CAGAATATAATTCTCAAACA | 6806-6825 |
| 107 | TAAAGTTCCTTTAAAGGCTGAGG | 6885-6907 |
| 108 | TTCTGGTATATTGGAAGAG | 6933-6951 |
| 109 | ATTTGTACCTACTCCAGATAATTCAG | 6963-6988 |
| 110 | TTATAGGTACATTAATTCAAAG | 6999-7020 |
| 111 | TGCAGTTGTTGAAAAAGAA | 7038-7056 |
| 112 | CTTTGCAAAATATACA | 7068-7083 |

HPV 39

| SEQ ID | | |
|---|---|---|
| NO | 5′→3′ | Locus in HPV 39 |
| 113 | CTCTATAGAGTCTTC | 6677-6691 |
| 114 | TAGAGTCTTCCATACCTTCT | 6682-6701 |
| 115 | ATAGAGTCTTCCATACCTTC | 6681-6700 |
| 116 | GTCTTCCATACCTTCTACATATG | 6686-6708 |
| 117 | CTACATATGATCCTTCTAAG | 6700-6719 |
| 118 | TACTGTCACATTAACAACTGATG | 6779-6801 |
| 119 | TTCCTCTATATTGGACAA | 6827-6844 |
| 120 | TGTAGCTCCTCCACCATCTGCCAGTT | 6857-6882 |
| 121 | TTACAGATACCTACAGTCTGCA | 6893-6914 |
| 122 | GGATGCTCCAGCACCTGAA | 6932-6950 |
| 123 | ATATGACGGTCTAAAG | 6962-6977 |

HPV 42

| SEQ ID | | |
|---|---|---|
| NO | 5′→3′ | Locus in HPV 42 |
| 124 | TATATGTTGGGGAAATCAGCTA | 6802-6823 |
| 125 | CACTGCAACATCTGGTGATA | 6874-6893 |
| 126 | GCAACATCTGGTGATACATATACAG | 6878-6907 |
| | CTGCT | |
| 127 | CATTAACTGTTGAAGTTATGTCA | 6978-7000 |
| 128 | CCTAACATATTAGAGGAGTGGAATG | 7019-7044 |
| | T | |
| 129 | CACCACCACCTTCAGGAACT | 7053-7072 |
| 130 | GTTATAGGTATGTACAATCAGAAG | 7083-7106 |
| 131 | GCTAAGGTAACAACGCCAGAAAAAA | 7121-7150 |
| | AGGAT | |
| 132 | CAGACTTTTGGTTTTGGGAGGTAA | 7158-7181 |
| 133 | GAAAAGTTTTCTACTGATTTA | 7190-7210 |

HPV 43

| SEQ ID | | |
|---|---|---|
| NO | 5′→3′ | Locus in HPV 43 |
| 134 | CATTTGTTTTGGGAATCAGTTG | 21-42 |
| 135 | TGACCCTACTGTGCCCAGTA | 99-118 |
| 136 | ACTGTGCCCAGTACATATGACAATGC | 106-135 |
| | AAAG | |
| 137 | GTTTATATTTCAATTATGCATAA | 177-199 |
| 138 | CCAGAGGTTATGACATATATT | 211-231 |
| 139 | CCCACATTATTAGAGGACTGGAA | 244-266 |
| 140 | CCACCTGCCTCTGCTTCTTTG | 280-300 |
| 141 | CGCTTTTTGTCTAACAAGGCCATTG | 313-337 |
| 142 | CCAAAGGAACGGGAGGATCCCTA | 358-380 |
| 143 | CTTACAGAAAAGTTTTCTGCACAAC | 409-433 |

HPV 44

| SEQ ID | | |
|---|---|---|
| NO | 5′→3′ | Locus in HPV 40 |
| 144 | TGCCACTACACAGTC | 6719-6733 |
| 145 | CTACACAGTCCCCTCCGTCT | 6724-6743 |
| 146 | TGCCACTACACAGTCCCCTC | 6719-6738 |
| 147 | CAGTCCCCTCCGTCTACATATA | 6729-6750 |
| 148 | CTACATATACTAGTGAACAA | 6742-6761 |
| 149 | TAGTATTACCTTAACGGCGGAGG | 6821-6843 |
| 150 | TGCTGGTATTTTAGAACAG | 6869-6887 |
| 151 | GTTGTCGCCGCCCCCAAATGGTACC | 6899-6924 |
| | T | |
| 152 | ATACAGATATGTGCAGTCCCAG | 6935-6956 |
| 153 | GCCACCCCCTGAAAAGGCA | 6974-6992 |
| 154 | CTATGCAAAATTAAGT | 7004-7019 |

HPV 45

| SEQ ID | | |
|---|---|---|
| NO | 5′→3′ | Locus in HPV 45 |
| 155 | TGCCTCTACACAAAATCCTG | 6651-6670 |
| 156 | CTCTACACAAAATCC | 6654-6668 |
| 157 | ACAAAATCCTGTGCCAAGTA | 6660-6679 |
| 158 | CAAAATCCTGTGCCAAGTAC | 6661-6680 |
| 159 | AATCCTGTGCCAAGTACATATG | 6664-6685 |
| 160 | GTACATATGACCCTACTAAG | 6677-6696 |
| 161 | CACTATTACTTTAACTGCAGAGG | 6756-6778 |
| 162 | TAGTAGTATATTAGAAAAT | 6804-6822 |
| 163 | TGTCCCTCCACCACCTACTACAAGTT | 6834-6859 |
| 164 | ATATCGTTTTGTGCAATCAGTT | 6870-6891 |
| 165 | GGATACTACACCTCCAGAA | 6909-6927 |

HPV 51

| SEQ ID | | |
|---|---|---|
| NO | 5′→3′ | Locus in HPV 51 |
| 166 | CACTGCCACTGCTGCGGTTT | 6555-6574 |
| 167 | TGCCACTGCTGCGGT | 6558-6572 |
| 168 | CACTGCTGCGGTTTCCCCAA | 6561-6580 |
| 169 | CCACTGCTGCGGTTTCCCCA | 6560-6579 |
| 170 | CTGCGGTTTCCCCAACATTTAC | 6566-6587 |
| 171 | CAACATTTACTCCAAGTAAC | 6578-6597 |
| 172 | TAAAATTACTTTAACTACAGAGG | 6657-6679 |
| 173 | TCCTACCATTCTTGAACAG | 6705-6723 |
| 174 | ATTAACATTACCTCCGTCTGCTAGTT | 6735-6760 |
| 175 | ATATAGGTTTGTTAGAAATGCA | 6771-6792 |
| 176 | GGACACCCCTCCACAGGCT | 6810-6828 |
| 177 | TTTGGCCAAATATAAA | 6840-6855 |

HPV 52

| SEQ ID | | |
|---|---|---|
| NO | 5′→3′ | Locus in HPV 52 |
| 178 | TGAGGTTAAAAAGGA | 6695-6709 |
| 179 | TGAGGTTAAAAAGGAAAGCA | 6695-6714 |
| 180 | GAGGTTAAAAAGGAAAGCAC | 6696-6715 |
| 181 | TTAAAAAGGAAAGCACATAT | 6700-6719 |
| 182 | AAAGGAAAGCACATATAAAAAT | 6704-6725 |
| 183 | GCACATATAAAAATGAAAAT | 6712-6731 |
| 184 | CAAAATTACATTAACAGCTGATG | 6791-6813 |
| 185 | TGCCACTATTTTAGAGGAC | 6839-6857 |
| 186 | CCTTACCCCACCACCGTCTGCATCTT | 6869-6894 |
| 187 | ATACAGATTTGTCACTTCTACT | 6905-6926 |
| 188 | AAACACACCACCTAAAGGA | 6944-6962 |
| 189 | TTTAAAGGACTATATG | 6974-6989 |

HPV 53

| SEQ ID | | |
|---|---|---|
| NO | 5′→3′ | Locus in HPV 53 |
| 190 | TCCGCAACCACACAGTCTAT | 6681-6700 |
| 191 | CCGCAACCACACAGT | 6682-6696 |
| 192 | CCGCAACCACACAGTCTATG | 6682-6701 |
| 193 | CACAGTCTATGTCTACATATAA | 6691-6712 |
| 194 | CTACATATAATTCAAAGCAA | 6703-6722 |
| 195 | TAAAATATCCCTGTCTGCTGAGG | 6782-6804 |
| 196 | TTCTACCTTACTGGAAGAC | 6830-6848 |
| 197 | TTTGTCGCCTCCTGTTGCCACTAGCT | 6860-6885 |
| 198 | ATACAGATATGTGAAAAGTGCA | 6896-6917 |
| 199 | GGATCAGCCCCCTCCTGAA | 6935-6953 |

HPV 54

| SEQ ID | | |
|---|---|---|
| NO | 5′→3′ | Locus in HPV 54 |
| 200 | TACAGCATCCACGCA | 6633-6647 |
| 201 | CAGCATCCACGCAGGATAGC | 6635-6654 |
| 202 | ACGCAGGATAGCTTTAATAA | 6643-6662 |
| 203 | CACGCAGGATAGCTTTAATA | 6642-6661 |
| 204 | ATAGCTTTAATAATTCTGAC | 6650-6669 |
| 205 | TACCATAACCCTTACAGCAGATG | 6729-6751 |
| 206 | TCCCACTATTCTAGAGGAC | 6777-6795 |
| 207 | TATAACCCCCCCAGCTACAAGTAGT | 6807-6832 |
| | T | |
| 208 | ATATAGGTTTGTACAGTCACAG | 6843-6864 |
| 209 | GAATAATGCCCCTGCAAAGGAA | 6882-6903 |

HPV 55

| SEQ ID | | |
|---|---|---|
| NO | 5′→3′ | Locus in HPV 55 |
| 210 | TTTGTTACTGTTGTAGATACTAC | 6669-6691 |
| 211 | ATGACAATATGTGCTGCTAC | 6705-6724 |
| 212 | GACAATATGTGCTGCTACAA | 6707-6726 |
| 213 | TGCTACAACTCAGTCTCCAT | 6719-6738 |
| 214 | CTACAACTCAGTCTCCATCT | 6721-6740 |
| 215 | ACAACTCAGTCTCCATCTAC | 6723-6742 |
| 216 | ATGTTGAGGAGTTTGACTTA | 6781-6800 |
| 217 | TGTTGAGGAGTTTGACTTAC | 6782-6801 |
| 218 | TGAGGAGTTTGACTTACAGT | 6785-6804 |

HPV 56

| SEQ ID | | |
|---|---|---|
| NO | 5′→3′ | Locus in HPV 56 |
| 219 | CTGCTACAGAACAGT | 6630-6644 |
| 220 | GCTACAGAACAGTTAAGTAA | 6632-6651 |
| 221 | CAGAACAGTTAAGTAAATAT | 6636-6655 |
| 222 | GAACAGTTAAGTAAATATGATGC | 6638-6660 |
| 223 | GTAAATATGATGCACGAAAA | 6648-6667 |
| 224 | CAAAATTACTTTGTCTGCAGAGG | 6727-6749 |
| 225 | TGCTAACCTACTGGAGGAC | 6775-6793 |
| 226 | GTTATCCCCGCCAGTGGCCACCAGCC | 6805-5830 |
| 227 | ATATAGATATGTTAGAAGCACA | 6841-6862 |
| 228 | GGAACAGCCACCAACAGAA | 6880-6898 |

HPV 58

| SEQ ID | | |
|---|---|---|
| NO | 5′→3′ | Locus in HPV 58 |
| 229 | ATGCACTGAAGTAACTAAGG | 6674-6693 |
| 230 | CACTGAAGTAACTAAGGAAG | 6677-6696 |
| 231 | TGAAGTAACTAAGGA | 6680-6694 |
| 232 | GAAGTAACTAAGGAAGGTAC | 6681-6700 |
| 233 | CTAAGGAAGGTACATATAAAAA | 6688-6709 |
| 234 | ATAAAAATGATAATTTTAAG | 6703-6722 |
| 235 | CAAAATTACACTAACTGCAGAGA | 6776-6798 |
| 236 | TTCCAATATTTTGGAGGAC | 6824-6842 |
| 237 | TTTAACACCTCCTCCGTCTGCCAGTT | 6854-6879 |
| 238 | ATATAGATTTGTTACCTCCCAG | 6890-6911 |
| 239 | AACAGCACCCCCTAAAGAA | 6929-6947 |

HPV 59

| SEQ ID | | |
|---|---|---|
| NO | 5′→3′ | Locus in HPV 59 |
| 240 | TTCTACTACTTCTTC | 6643-6657 |
| 241 | ACTACTTCTTCTATTCCTAA | 6647-6666 |
| 242 | ACTTCTTCTATTCCTAATGT | 6650-6669 |
| 243 | TCTTCTATTCCTAATGTATACAC | 6653-6675 |
| 244 | ATGTATACACACCTACCAGT | 6666-6685 |
| 245 | TAAAATAACATTAACTACAGAGG | 6745-6767 |
| 246 | TACCACTATTTTGGAGGAT | 6793-6811 |
| 247 | TGTTACACCACCTCCTACTGCTAGTT | 6823-6848 |
| 248 | ATACCGTTTTGTTCAATCTGCT | 6859-6880 |
| 249 | GGACACCGCACCGCCAGTT | 6898-6916 |
| 250 | TTATGACAAACTAAAG | 6928-6943 |

HPV 61

| SEQ ID | | |
|---|---|---|
| NO | 5′→3′ | Locus in HPV 61 |
| 251 | CTGCTACATCCCCCC | 6803-6817 |
| 252 | ACATCCCCCCCTGTATCTGA | 6808-6827 |
| 253 | CATCCCCCCCTGTATCTGAA | 6809-6828 |
| 254 | CCCCTGTATCTGAATATAAAGC | 6815-6836 |
| 255 | CTGAATATAAAGCCACAAGC | 6824-6843 |
| 256 | TAAAATACATTTAACCCCTGAAA | 6903-6925 |
| 257 | TAAGGCCTTGTTGGATGAC | 6951-6969 |
| 258 | TGTGGTACCACCACCCTCTACCAGTT | 6981-7006 |
| 259 | ATATAGGTTTTTGCAGTCCAGA | 7017-7038 |
| 260 | GGGTGCTGCTGCCCCGCCGCCC | 7056-7077 |
| 261 | CTATGCCAAGTTATCC | 7089-7104 |

HPV 62

| SEQ ID | | |
|---|---|---|
| NO | 5′→3′ | Locus in HPV 62 |
| 262 | CCGCCTCCACTGCTG | 92-106 |
| 263 | GCCTCCACTGCTGCAGCAGA | 94-113 |
| 264 | CTGCTGCAGCAGAATACACG | 101-120 |
| 265 | GCAGAATACACGGCTACCAA | 109-128 |
| 266 | CAGAATACACGGCTACCAAC | 110-129 |
| 267 | CAAAATACAGTTAACCCCCGAAA | 189-211 |
| 268 | CAAGGACCTTTTGGATGAC | 237-255 |
| 269 | GGTTTTACCTCCCCCTTCCACTAGTT | 267-292 |
| 270 | ATATCACTATTTCGAGTCTCGG | 303-324 |
| 271 | GGGGCTGCCTACCCGTCCC | 342-360 |
| 272 | GTATGCGCAAATGACA | 372-387 |

HPV 66

| SEQ ID | | |
|---|---|---|
| NO | 5′→3′ | Locus in HPV 66 |
| 273 | CAGCTAAAAGCACAT | 6680-6694 |
| 274 | CAGCTAAAAGCACATTAACT | 6680-6699 |
| 275 | CTAAAAGCACATTAACTAAA | 6683-6702 |
| 276 | TTAACTAAATATGATGCCCG | 6694-6713 |
| 277 | CTAAATATGATGCCCGTGAA | 6698-6717 |
| 278 | TAAAATAACCTTAACTGCAGAAG | 6777-6799 |
| 279 | TAATACTTTATTAGACGAT | 6825-6843 |
| 280 | CTTATCCCCACCAGTTGCAACTAGCT | 6855-6880 |
| 281 | ATATAGGTATATTAAAAGCACA | 6891-6912 |
| 282 | GGAACAGCCCCCTGCAGAA | 6930-6948 |
| 283 | CCTGGCTAAATATAAG | 6960-6975 |

HPV 67

| SEQ ID | | |
|---|---|---|
| NO | 5′→3′ | Locus in HPV 67 |
| 284 | CTGAGGAAAAATCAG | 6655-6669 |
| 285 | GAGGAAAAATCAGAGGCTAC | 6657-6676 |
| 286 | ATCAGAGGCTACATACAAAAATG | 6665-6687 |
| 287 | AGGAAAAATCAGAGGCTACA | 6658-6677 |
| 288 | CTACATACAAAAATGAAAAC | 6673-6692 |
| 289 | CAAAATATCCCTTACTGCAAATG | 6752-6774 |
| 290 | TCCAGATATATTAGAGGAC | 6800-6818 |
| 291 | CCTTACACCACCTCCTTCAGGTAATT | 6830-6855 |
| 292 | ATATAGATTTGTTACCTCGCAG | 6866-6887 |
| 293 | AACATCCCCTCCAACAGCA | 6905-6923 |
| 294 | TCTTAAAAAGTACAGT | 6935-6950 |

HPV 68

| SEQ ID | | |
|---|---|---|
| NO | 5′→3′ | Locus in HPV 68 |
| 295 | CTACTACTGAATCAG | 2653-2667 |
| 296 | TGAATCAGCTGTACCAAATA | 2660-2679 |
| 297 | GAATCAGCTGTACCAAATAT | 2661-2680 |
| 298 | CAGCTGTACCAAATATTTATGA | 2665-2686 |
| 299 | ATATTTATGATCCTAATAAA | 2677-2696 |
| 300 | TCCTGCTATTTTGGATGAT | 2804-2822 |
| 301 | TACTATAACATTGTCCACTGATG | 2756-2778 |
| 302 | TGTTGCCCCTCCACCATCTGCTAGTC | 2834-2859 |
| 303 | ATACCGCTATCTGCAATCAGCA | 2870-2891 |
| 304 | AGACGCCCCTGCACCTACT | 2909-2927 |
| 305 | ATATGATGGCTTAAAC | 2939-2954 |

HPV 69

| SEQ ID | | |
|---|---|---|
| NO | 5′→3′ | Locus in HPV 69 |
| 306 | TATTAGTACTGTATCTGCAC | 6572-6591 |
| 307 | CTGTATCTGCACAAT | 6580-6594 |
| 308 | CTGTATCTGCACAATCTGCA | 6580-6599 |
| 309 | TGCACAATCTGCATCTGCCA | 6587-6606 |
| 310 | CAATCTGCATCTGCCACTTTTA | 6591-6612 |
| 311 | CCACTTTTAAACCATCAGAT | 6604-6623 |
| 312 | TAAAATTACTCTTACCACTGATG | 6683-6705 |
| 313 | TTCTACTATTTTGGAAAAT | 6731-6749 |
| 314 | CCTTACCTTGCCTCCTACTGCTAGT | 6761-6786 |
| | T | |
| 315 | ATATAGGTTTATTAAAAATTCA | 6797-6818 |
| 316 | CGATGCCCCTGCACAGCCC | 6836-6854 |

HPV 70

| SEQ ID | | |
|---|---|---|
| NO | 5′→3′ | Locus in HPV 70 |
| 317 | TGTCTGCCTGCACCGAAACG | 6614-6633 |
| 318 | CTGCACCGAAACGGC | 6621-6635 |
| 319 | GAAACGGCCATACCTGCTGT | 6628-6647 |
| 320 | CGAAACGGCCATACCTGCTG | 6627-6646 |
| 321 | CGGCCATACCTGCTGTATATAG | 6632-6653 |
| 322 | CTGTATATAGCCCTACAAAG | 6644-6663 |
| 323 | TACTATCACATTAACTGCTGACG | 6723-6745 |
| 324 | TCCTGCAATTTTGGACAAT | 6771-6789 |
| 325 | AGTTACCCCTCCACCATCTGCAAG | 6801-6826 |
| | CT | |
| 326 | GTATAGGTATTTACAATCAGCA | 6837-6858 |
| 327 | GGATGCTCCTACACCTGAA | 6876-6894 |
| 328 | CTATGACGATTTAAAA | 6906-6921 |

HPV 72

| SEQ ID | | |
|---|---|---|
| NO | 5′→3′ | Locus in HPV 72 |
| 329 | ATCTGTTGGTTTAATGAGCT | 6759-6778 |
| 330 | TTTGTGACAGTTGTAGATAC | 6780-6799 |
| 331 | CTGCCACAGCGTCCT | 6829-6843 |
| 332 | ACAGCGTCCTCTGTATCAGA | 6834-6853 |
| 333 | CCACAGCGTCCTCTGTATCA | 6832-6851 |
| 334 | AGCGTCCTCTGTATCAGAATAT | 6836-6857 |
| 335 | CAGAATATACAGCTTCTAAT | 6850-6869 |
| 336 | TAAAATTCACTTAACTCCTGAAA | 6929-6951 |
| 337 | TAAGGCCTTATTGGATGAC | 6977-6995 |
| 338 | TGTGGTGCCTCCTCCTTCTACCAGTT | 7007-7032 |
| 339 | CTATAGGTTTTTGCAGTCTCGT | 7043-7064 |
| 340 | GGGGGCTGCCACCCCTCCTCCT | 7082-7103 |
| 341 | ATATGCTAACTTATCC | 7115-7130 |

HPV 74

| SEQ ID | | |
|---|---|---|
| NO | 5′→3′ | Locus in HPV 74 |
| 342 | CCTACCTCACAATCG | 1686-1700 |
| 343 | CTCACAATCGCCTTCTGCTA | 1691-1710 |
| 344 | ACCTCACAATCGCCTTCTGC | 1689-1708 |
| 345 | CAATCGCCTTCTGCTACATATA | 1695-1716 |
| 346 | ACAATCGCCTTCTGCTACATAT | 1694-1715 |
| 347 | CTACATATAATAGTTCAGAC | 1708-1727 |
| 348 | TAGTATTAAGTTAACTGCTGAGG | 1787-1809 |
| 349 | TCCTACAGTTTTAGAAGAG | 1835-1853 |
| 350 | GCTAACGCCTCCCCCCAATGGTACTT | 1865-1890 |
| 351 | CTACAGATATGTGCAGTCCCAG | 1901-1922 |
| 352 | ACCTACGCCTGATAAAGCA | 1940-1958 |
| 353 | CTATGCAAATTTAAGT | 1970-1985 |

HPV 82

| SEQ ID | | |
|---|---|---|
| NO | 5′→3′ | Locus in HPV 82 |
| 354 | TGCTGTTACTCCATC | 6608-6622 |
| 355 | TGCTGTTACTCCATCTGTTG | 6608-6627 |
| 356 | ACTCCATCTGTTGCACAAAC | 6615-6634 |
| 357 | AAACATTTACTCCAGCAAAC | 6631-6650 |
| 358 | TAAAATCACTTTAACTACTGAAA | 6710-6732 |
| 359 | TTCTACAATTTTAGAACAG | 6758-6776 |
| 360 | ATTAACATTGCCCCCCTCCGCTAGTT | 6788-6813 |
| 361 | CTATCGATTTGTAAAAAATGCA | 6824-6845 |
| 362 | GGACAGTCCTCCACAGGCT | 6863-6881 |

HPV CP8061

| SEQ ID | | Locus in HPV |
|---|---|---|
| NO | 5′→3′ | CP8061 |
| 363 | TCTGTGCTACCAAAACTGTT | 86-105 |
| 364 | CTACCAAAACTGTTG | 92-106 |
| 365 | ACCAAAACTGTTGAGTCTAC | 94-113 |
| 366 | AACTGTTGAGTCTACATATAAA | 99-120 |
| 367 | GTTGAGTCTACATATAAAGC | 103-122 |
| 368 | CTACATATAAAGCCTCTAGT | 110-129 |
| 369 | TGTTATTAATTTAACAGCTGAAA | 189-211 |
| 370 | TGCTACATTACTGGAGGAC | 237-255 |
| 371 | GTTCTTACCACCTCCTACTG | 267-286 |
| 372 | CTACCGCTTTTTACAGTCTCAG | 303-324 |
| 373 | AAACAGTCCTCCTCCTGCAGAA | 342-363 |
| 374 | CTATGCAGATCTTACA | 375-390 |

HPV CP8034

| SEQ ID | | Locus in HPV |
|---|---|---|
| NO | 5′→3′ | CP8034 |
| 375 | CAGCTACATCTGCTG | 92-106 |
| 376 | GCTACATCTGCTGCTGCAGA | 94-113 |
| 377 | ACATCTGCTGCTGCAGAATACA | 97-118 |
| 378 | TGCTGCAGAATACAAGGCCT | 105-124 |
| 379 | GCTGCAGAATACAAGGCCTC | 106-125 |
| 380 | CAGAATACAAGGCCTCTAAC | 110-129 |
| 381 | TAAAATACAGTTAACACCAGAAA | 189-211 |
| 382 | CAAGGCACTGTTGGATGAT | 237-255 |
| 383 | TGTGTTGCCACCTCCTTCCACCAGTT | 267-292 |
| 384 | ATATCGCTTTTTACAGTCTCGG | 303-324 |
| 385 | GGGTGCTGCTGCCCCTGCGCCC | 342-363 |
| 386 | TTATGCCGACATGTCA | 375-390 |

HPV L1AE5

| SEQ ID | | Locus in HPV |
|---|---|---|
| NO | 5′→3′ | L1AE5 |
| 387 | ATCTACTGCAACTACTAATC | 69-88 |
| 388 | CTGCAACTACTAATC | 74-88 |
| 389 | CTGCAACTACTAATCCAGTT | 74-93 |
| 390 | ACTACTAATCCAGTTCCATCTA | 79-100 |
| 391 | CTAATCCAGTTCCATCTATA | 83-102 |
| 392 | CTATATATGAACCTTCTAAA | 98-117 |
| 393 | TAAAATTACACTTACTACTGATG | 177-199 |
| 394 | TCCTACTATTTTAGATAGT | 225-243 |
| 395 | TGTTAGTCCTCCCCCATCTGCTAGCT | 255-280 |
| 396 | ATATAGGTTTTTACAGTCATCT | 291-312 |
| 397 | GGATGTGGTTGTTCCACAA | 330-348 |

HPV MM4

| SEQ ID | | Locus in HPV |
|---|---|---|
| NO | 5′→3′ | MM4 |
| 398 | CTGCTGTTACTCAATCTGTT | 92-111 |
| 399 | TGCTGTTACTCAATC | 93-107 |
| 400 | GTTACTCAATCTGTTGCACA | 97-116 |
| 401 | TGCACAAACATTTACTCCAG | 111-130 |
| 402 | TTACTCAATCTGTTGCACAAAC | 98-119 |
| 403 | AAACATTTACTCCAGCAAAC | 116-135 |
| 404 | TAAAATCACTTTAACTACTGAAA | 195-217 |
| 405 | TTCTACAATTTTAGAACAG | 243-261 |
| 406 | ATTAACCTTGCCCCCCTCAGCTAGTT | 273-298 |
| 407 | CTATCGATTTGTAAAAAATGCA | 309-330 |
| 408 | GGACAGTCCTCCACAGGCT | 348-366 |

HPV MM7

| SEQ ID | | Locus in HPV |
|---|---|---|
| NO | 5′→3′ | MM7 |
| 409 | TGCTGCTACACAGGC | 93-107 |
| 410 | GCTGCTACACAGGCTAATGA | 94-113 |
| 411 | TGCTACACAGGCTAATGAAT | 96-115 |
| 412 | CTACACAGGCTAATGAATACAC | 98-119 |
| 413 | ATGAATACACAGCCTCTAAC | 110-129 |
| 414 | CAAAATACATCTTACCCCTGAAA | 189-211 |
| 415 | TGAACATTTATTGGATGAG | 237-255 |
| 416 | CGTGTTACCACCTCCTTCCACCAGCC | 267-292 |
| 417 | CTATCGCTATCTGCAGTCCCGT | 303-324 |
| 418 | GGGTCCTTCCGCCCCTGCCCCT | 342-363 |
| 419 | TTATGATGGCCTTGTA | 375-390 |

HPV MM8

| SEQ ID | | Locus in HPV |
|---|---|---|
| NO | 5′→3′ | MM8 |
| 420 | TGCTACCAACACCGA | 93-107 |
| 421 | CTACCAACACCGAATCAGAA | 95-114 |
| 422 | CCAACACCGAATCAGAATATAA | 98-119 |
| 423 | CAGAATATAAACCTACCAAT | 110-129 |
| 424 | TAAGGTCCGTCTGACTCCAGAGG | 189-211 |
| 425 | TGACTCCTTATTAGATGAG | 237-255 |
| 426 | TGTTGTGCCCCCTCCCTCCACAAGTT | 267-292 |
| 427 | CTATAGGTACTTGCAGTCTCGC | 303-324 |
| 428 | GGGGGCCGCCGCCGCCAAGCCT | 342-363 |
| 429 | TTATGCTGGCATGTCC | 375-390 |

The sequences of the probes listed above are either identical or complementary to the corresponding sequences of HPV subtypes so that the probes can hybridize with the sequences of HPV subtypes perfectly.

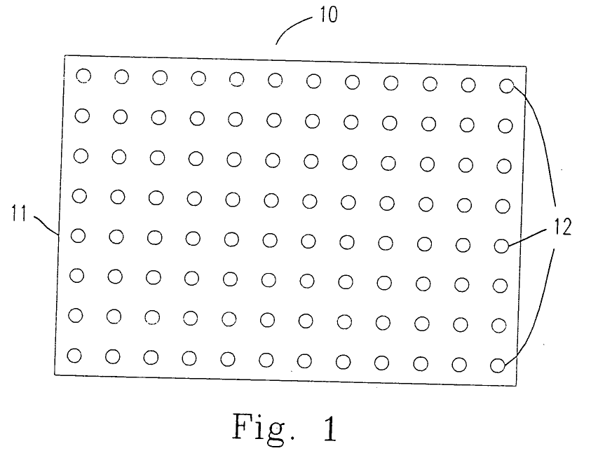

According to a preferred embodiment of the present invention, a detector for detecting and simultaneously diagnosing 39 subtypes of human papilloma viruses (HPV) contained in a biological sample is provided. Please refer to FIG. 1. The detector 10 is an oligonucleotide biochip. The detector 10 includes a carrier 11 and a plurality of micro-dots 12 immobilized on the carrier 11. The carrier 11 is a nylon membrane. Each micro-dot 12 is used for identifying one particular HPV subtype. There is at least one oligonucleotide sequence contained in each micro-dot 12 that is specific to one particular HPV subtype. The oligonucleotide sequences are the probes selected from the above list for each HPV subtype respectively. For example, the probe on the carrier 11 could contain at least one sequence, which is selected from SEQ ID NO 1 to SEQ ID NO 12 (shown above), for identifying the subtype 6 of human papilloma viruses (HPV 6).

As described in the above, the probes will hybridize specifically with the L1 gene sequence of the corresponding HPV subtype. Preferably, the probes have a length between 15-30 bases. The oligonucleotide sequences contained in each micro-dot 12 serve as a detection probe, which hybridizes specifically with the L1 gene sequence of the particular HPV subtype to form a hybridization complex as a detection indicator. Therefore, each micro-dot 12 identifies a specific HPV subtype via a corresponding oligonucleotide of the specific HPV subtype, and thereby detecting and simultaneously identifying subtypes of human papilloma viruses. The sequences of the oligonucleotides provided by the present invention are specific to the epidemics of human papilloma viruses. The detector 10 is able to simultaneously identify 39 different HPV subtype that are HPV 6, HPV 11, HPV 16, HPV 18, HPV 26, HPV 31, HPV 32, HPV 33, HPV 35, HPV 37, HPV 39, HPV 42, HPV 43, HPV 44, HPV 45, HPV 51, HPV 52, HPV 53, HPV 54, HPV 55, HPV 56, HPV 58, HPV 59, HPV 61, HPV 62, HPV 66, HPV 67, HPV 68, HPV 69, HPV 70, HPV 72, HPV 74, HPV 82, HPV CP8061, HPV CP8034, HPV L1AE5, HPV MM4, HPV MM7 and HPV MM8. Furthermore, the detector 10 includes the micro-dot 12 containing a Glutaldehyde-3-phosphodehydrogenase (GAPDH) gene, which is used as an internal control.

### EXAMPLE I

The method for immobilizing or mounting the above mentioned probes (oligonucleotides) on the carrier 11 (the nylon membrane) is described as follows.

1.-TTTTTTTTTTTTTTT (SEQ ID NO 469) is added to the 3′ end of the oligonucleotide provided by the present invention by terminal transferase according to the following steps 1.1 to 1.3.

1.1 Mixing the following components:

| | | |
|---|---|---|
| 10X NEBuffer 4 | 5 | μl |
| 2.5 mM CoCl₂ | 5 | μl |
| oligonucleotide | 5˜300 | pmol |
| 10˜300 mM dATP, dCTP, dTTP or dGTP | 1 | μl |
| Terminal Transferase (20 U/μl) | 0.5˜5 | μl |
| (NEW English BioLabs, M0252S) | | |
| Add M.Q. H₂O to final volume | 50 | μl |

1.2 The components are mixed at 37° C. for 15-60 minutes.

1.3 10 μl of 0.2 M EDTA (pH 8.0) is added to the mixture to stop the reaction.

2. The oligonucleotide having 3′ end labeling is mounted on the carrier 11 according to the following steps 2.1 to 2.3.

2.1 The oligonucleotide having 3′ end labeling is mounted on the carrier 11 by a needle having a 400 μm wide head. The distance between each dot is 1200 μm.

2.2 The carrier 11 having the dot array 12 thereon is exposed to UV light, and the detector 10 is formed.

2.3 The detector 10 is preserved in a drying box.

### EXAMPLE II

According to another preferred embodiment of the present invention, the carrier 11 could be a glass plate. The method for immobilizing or mounting the above mentioned probes (oligonucleotides) on the carrier 11 (glass plate) is described as follows.

1. The surface of the carrier 11 is treated according to the following steps 1.1 to 1.8.

1.1 The carrier 11 is cleaned in non-fluorescent and soft cleaner.

1.2 The clean carrier 11 is immersed in 10% NaOH.

1.3 The carrier 11 is oscillated in double-distilled water, 1% HCl solution and methanol in sequence for 2 minutes, and dried in an oven.

1.4 The carrier 11 is immersed in 1% 3-aminopropyltrimethoxysilane (APTMS) in 95% aqueous acetone at room temperature for about 2 minutes.

1.5 The carrier 11 is washed in acetone, and the carrier 11 is dried in the oven at 110° C. for 45 minutes.

1.6 The dried carrier 11 is immersed in 0.2% 1,4-phenylene diisothiocyanate, wherein the solvent is 10% pyridine in dimethyl formamide), at room temperature for 2 hours.

1.7 The carrier 11 is washed in methanol and acetone, and then the carrier 11 is dried.

1.8 The dried carrier 11 is preserved in a vacuum and dry box.

2. The oligonucleotides provided by the present invention are mounted on the carrier 11 (the glass plate) according to the following steps 2.1 to 2.3.

2.1 The oligonucleotide having 3′ end labeling is mounted on the carrier 11 by a needle having a 400 μm wide head. The distance between each dot is 1200 μm.

2.2 The carrier 11 is immersed in 1% NH₄OH solution for about 2 minutes, washed in double-distilled water, and then dried at room temperature. Thus, the detector 10 is formed.

2.3 The detector 10 is preserved in a dried box.

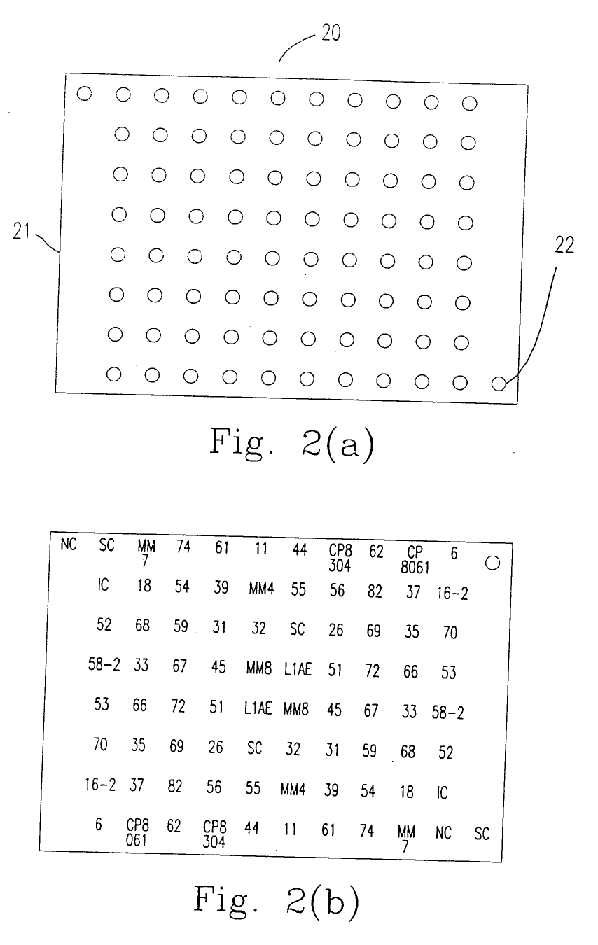

According to the above description, a biochip for specifically identifying the subtypes of human papilloma viruses contained in a biological sample is provided. Please refer to FIG. 2(a). The biochip 20 includes a carrier 21 and a plurality of micro-dots 22 immobilized on the carrier 21. The carrier 21 is a nylon membrane. The actual length of the nylon membrane is about 1.44 cm and the actual width of the nylon membrane is about 0.96 cm. The micro-dots 22 are mounted on the carrier 21 according to the foresaid method, wherein the distance between each dot is about 1.2 mm and the diameter of each dot is about 0.4 mm. Each micro-dot 22 contains at least one oligonucleotide (15˜30 mer), and each micro-dot 22 is used for specifically identifying a specific HPV subtype. The sequence of the oligonucleotide is selected from the foresaid list.

The subtype of human papilloma viruses identified by each dot of the micro-dots 22 is illustrated in FIG. 2(b). SC (system control) presents the PCR product amplified from any subtype of human papilloma viruses and biotin-contained primer. NC (negative control) presents the plants DNA fragment irrelevant to HPV. IN (internal control) presents the sequence 5′-gcccagactgtgggtggcag-3′ (SEQ ID NO 470) of the housekeeping gene, Glyceraldehyde-3-Phosphate Dehydrogenase (GAPDH). In sum, the biochip 20 provided in the present invention is able to detect and simultaneously identify 39 different HPV subtypes contained in the biological sample.

According to another preferred embodiment of the present invention, a method for detecting and simultaneously diagnosing 39 subtypes of human papilloma viruses (HPV) contained in a biological sample is provided. The steps are generally described as follows. First, the L1 gene fragment of human papilloma viruses (HPV) contained in the biological sample is amplified by polymerase chain reaction (PCR) using primers labeled with signaling substance. After the amplification product is obtained, it is hybridized with the detector 11 as describe above to form a hybridization complex. Then, the nonhybridized amplification product is removed from the detector 11. Next, the detector 11 is detected for the existence of the hybridization complex through detecting the signaling substance. The micro-dot 12 having the signaling substance shown thereon means a positive result that the biological sample contains the specific HPV subtypes recognized by the corresponding micro-dot 12. Ultimately, the HPV subtypes contained in the biological sample are thereby detected and simultaneously identified.

The method provided by the present invention for detecting and simultaneously identifying 39 subtypes of human papilloma viruses contained in a sample is described as follows.

### EXAMPLE III

1. The biological sample obtained from the patient is treated according to the following steps 1.1 to 1.3.

1.1 The cells are centrifuged at 1,500 rpm at 200□ for 5 minutes.

1.2 The cell pellet is washed in 10 mM Tris (pH 8.5) and dissolved in 8 mM NaOH. Then, the solution is transfer to 1.5 mL micro-tube.

1.3 A proper amount of TreTaq (1U/μl) solution is added to the micro-tube. The reaction is carried out at 95□ for 1 hour. The DNA contained in the sample is obtained after centrifugation at 13,500 rpm, 20□ for 5 minutes. The obtained DNA is preserved at −20□.

### EXAMPLE IV

2 The L1 gene fragment of human papilloma viruses (HPV) contained in the biological sample is then amplified by polymerase chain reaction (PCR). The polymerase chain reactions are performed according to the following steps.

2.1 Glutaldehyde-3-phosphodehydrogenase (GAPDH) gene is used as the internal control of the polymerase chain reactions so that it could help confirm whether the detecting protocols are precisely followed. The steps are described according to the following steps 2.1.1 to 2.1.3.

2.1.1 Mixing the following components:

| Reagent | Stock | | amount | Final concentration | |
|---|---|---|---|---|---|
| Sterile H₂O | | | 2.6 | | |
| 10X Taq Buffer | | | 0.5 | 1X Taq Buffer | |
| dNTP | 2.5 | mM | 0.4 | 200 | μM |
| Template | | | 1 | | |
| GAP241-5¹⁾ primer | 10 | pmol/μl | 0.2 | 0.4 | pmol/μl |
| GAP241-3²⁾ primer | 10 | pmol/μl | 0.2 | 0.4 | pmol/μl |
| ProTaq (PROTECH) | 5 | U/μl | 0.1 | 0.1 | U/μl |
| Total volume (μl) | | | 5 | | |

¹⁾Gap241-5 (SEQ ID NO 471): CCACCAACTGCTTAGCACCCC
²⁾Gap241-3 (SEQ ID NO 472): TGCAGCGTACTCCCCACATCA
3) The proper amount of mineral oil is added to prevent the evaporation.

2.1.2 The polymerase chain reaction is performed according to the following programs.

| | | |
|---|---|---|
| Program 1 | Program 2 | Program 3 |

94° C., 15 seconds

| | | |
|---|---|---|
| 94° C., | 57° C., | 72° C., |
| 3 minutes | 1 minute | 5 minutes |

72° C., 30 seconds
40 cycles

2.1.3 The product of the polymerase chain reaction is analyzed in 2.5% agarose/EtBr (0.5×TBE).

2.2 The DNA contained in the sample is amplified by the polymerase chain reaction according to the following steps.

2.2.1 Mixing the following components:

| Reagent | Stock | | Amount | Final concentration | |
|---|---|---|---|---|---|
| Sterile H₂O | | | 4.7-5.7 | | |
| 10X Taq Buffer | | | 1 | 1× Taq Buffer | |
| dNTP | 2.5 | mM | 0.8 | 200 | μM |
| Template | | | 1-2 | | |
| BSA | 10 | mg/ml | 0.1 | 0.1 | μg/μl |
| Primer^{1,2)} | 10 | pmol/μl | 0.6 | 0.6 | pmol/μl |
| Primer^{1,2)} | 10 | pmol/μl | 0.6 | 0.6 | pmol/μl |
| ProTaq (PRO_{TECH}) | 5 | U/μl | 0.2 | 0.1 | U/μl |
| Total volume (μl) | | | 10 | | |

¹⁾MY09/MY11: Weimin et al., 1997, J. Clin. Microbiol. 35(6): 1304-1310
²⁾MY11/GP6+: Weimin et al., 1997, J. Clin. Microbiol. 35(6): 1304-1310
3) The proper amount of mineral oil is added to prevent the evaporation.
4) The 5' end of the MY09 and GP6+ primers could be labeled with biotin or Cy5 fluorescent substances.

2.2.2 The polymerase chain reaction is performed according to the following programs.

| | | |
|---|---|---|
| Program 1 | Program 2 | Program 3 |

94° C., 45 seconds

| | | |
|---|---|---|
| 94° C., | 45° C., | 72° C., |
| 3 minutes | 1 minute | 5 minutes |

72° C., 1.5 minutes
45 cycles

2.2.3 The product of the polymerase chain reaction is analyzed in 2.5% agarose/EtBr (0.5×TBE).

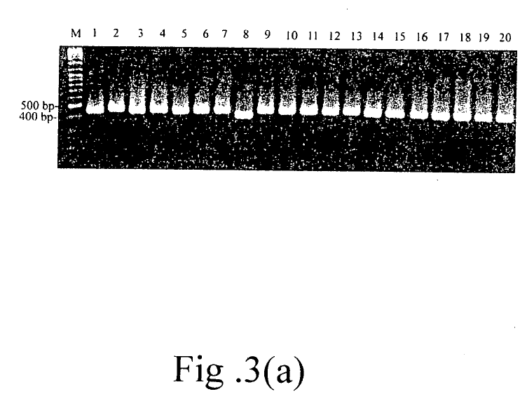

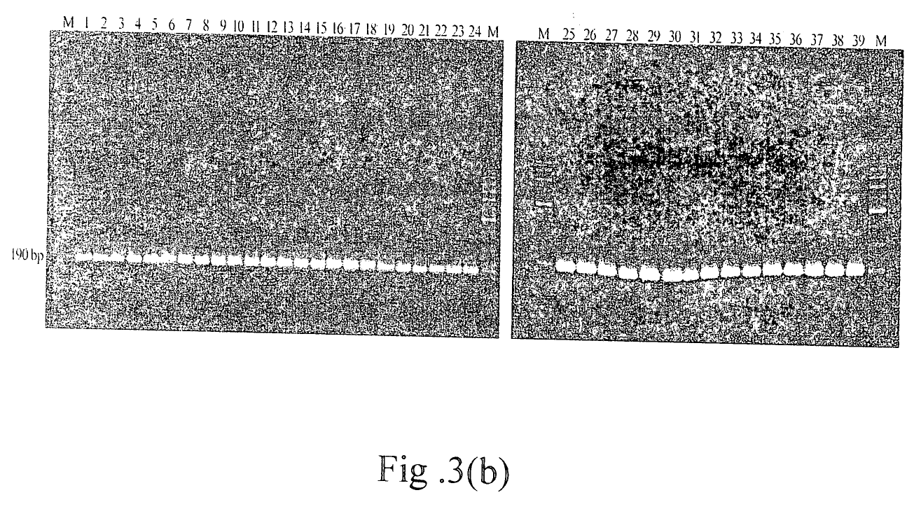

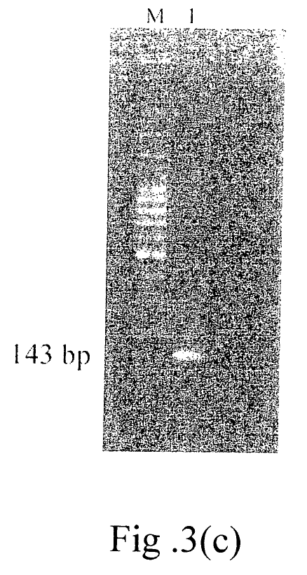

According to the above description, the biochip 20 is used for identifying different HPV subtypes. In one embodiment of the invention, the positive clones of human papilloma viruses are used and detected according to the foresaid method. As previously mentioned, the PCR amplification product could be obtained by different primer sets. One is primer set MY09/MY11, the other is primer set MY11/GP6+. Therefore, the positive clones are respectively amplified by PCR using MY11/MY09 primers and MY11/GP6+ primers. The products of the polymerase chain reaction are analyzed in 2.5% agarose/EtBr, and the electrophoresis results are shown in FIG. 3(a)-(c). FIG. 3(a) shows the electrophoresis result of the analyzed PCR products using primer set MY09/MY11. In FIG. 3(a), M presents DNA marker. Lane 1˜20 present HPV 6, HPV 11, HPV 16, HPV 18, HPV 26, HPV 31, HPV 33, HPV 35, HPV 44, HPV 45, HPV 52, HPV 53, HPV 54, HPV 56, HPV 59, HPV 61, HPV 66, HPV 70, HPV CP8061, and HPV L1AE5 in sequence. FIG. 3(b) shows the electrophoresis result of the analyzed PCR products using primer set MY11/GP6+. In FIG. 3(b), M presents DNA marker. Lane 1˜39 present HPV 6, 11, 16,18, 26, 31, 32, 33, 35, 37, 39, 42, 43, 44, 45, 51, 52, 53, 54, 56, 58, 59, 61, 62, 66, 67, 68, 69, 70, 72, 74, 82, CP8061, CP8304, L1AE5, MM4, MM7, and MM8 in sequence. FIG. 3(c) shows the electrophoresis result of the PCR products using GAPDH primer set. Clearly, the electrophoresis results show the PCR products with correct sizes. That is, PCR products using primer set MY09/MY11 is about 450 bp, the PCR products using primer set MY11/GP6+ is about 190 bp, and the PCR products using GAPDH primer set is about 190 bp.

### EXAMPLE V

3. When the carrier 11 is a nylon membrane, the detector 10 provided by the present invention is used for identifying the subtypes of human papilloma viruses according to the following hybridization steps.

3.1 The detector 10 is immersed in 2×SSC solution for 5 minutes.

3.2 The detector 10 is immersed in a buffer containing salmon sperm DNA (50 μg/μl), and the oligonucleotides mounted on the detector 10 are pre-hybridized with the salmon sperm DNA at 35□ for 30 minutes.

3.3 The PCR product having biotin labeled thereon is added into and mixed with a buffer containing salmon sperm DNA (50 μg/μl) at 95□ for about 5 minutes. The denatured DNA is placed on ice.

3.4 The denature DNA is added to the detector 10 and hybridized with the oligonucleotides at 35□ for 4 hours or overnight.

3.5 The detector 10 is washed in 2×SSC/1% SDS solution at 35□ for 15 minutes.

3.6 The detector 10 is washed in 0.2×SSC/0.1% SDS solution at 35□ for 15 minutes.

3.7 The detector 10 is treated in 0.5% isolation reagent for 1 hour.

3.8 The detector 10 is treated with avidin-alkalinephosphatase for about 1 hour.

3.9 The detector 10 is washed in 1×PBST solution.

3.10 The detector 10 is washed in Tris/NaCl solution.

3.11 The detector 10 is treated with NBT/BCIP at room temperature to show the reacting dot in blue.

3.12 The blue dot having the specific oligonucleotide sequence presents the specific subtype of human papilloma viruses contained in the sample.

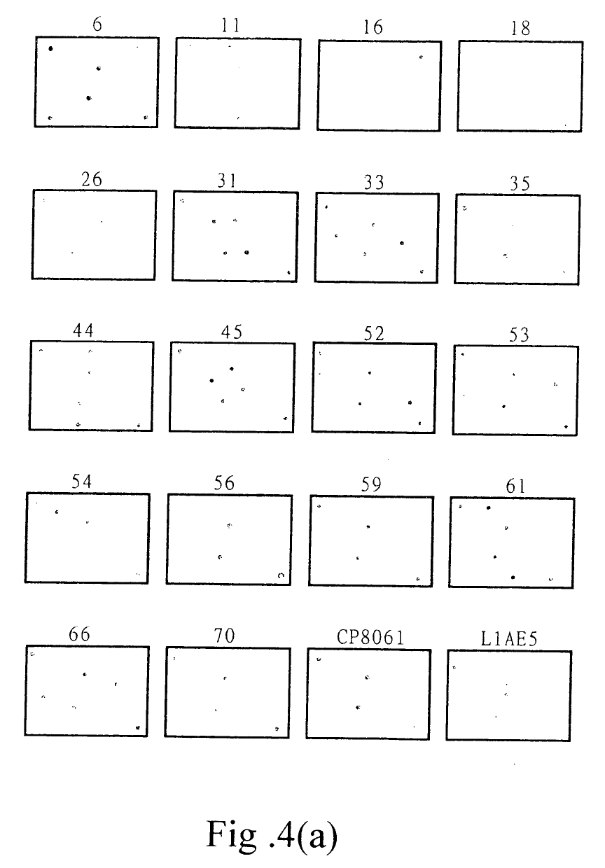

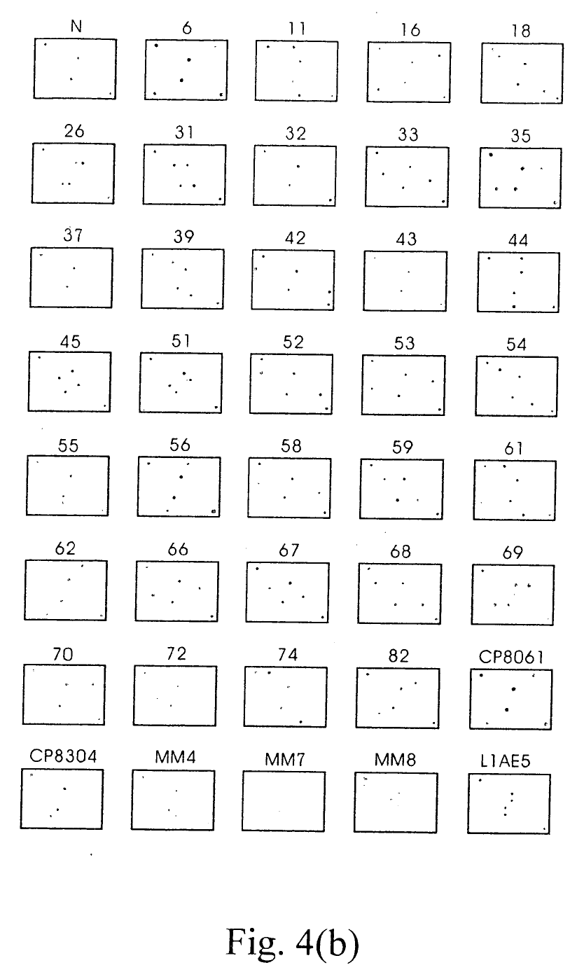

Preferably, the foresaid PCR amplified products shown in FIGS. 3(a)and 3(b) are then respectively detected by the biochip 20 according to the above steps and the results are shown in FIGS. 4(a) and 4(b). FIG. 4(a) shows the detecting result of detecting the PCR products using primer set MY09/MY11 of HPV positive clones. FIG. 4(b) shows the detecting result of detecting the PCR products using primer set MY11/GP6+ of HPV positive clones. When comparing the results shown in FIG. 4(a) and FIG. 3(b) based on the “SC” dot, it is very clear that the biochip 20 can precisely identify the subtype of human papilloma viruses. Take the result of HPV 6 as example. Since this biochip is hybridized with the PCR product amplified from HPV 6 positive clone, there should be 6 positive micro-dots shown on the biochip 20, including 2 SC micro-dots at the corners, 2 SC micro-dots in the central, and 2 micro-dots of HPV 6. The result clearly shows the exact 6 positive micro-dots without any other false positive micro-dot. Obviously, all the results of other biochips in FIGS. 4(a) and 4(b) show a clear and clean result as well. In other words, there is no cross reaction occurred in the detection, which proves that the biochip provided in the present invention has a very high specificity.

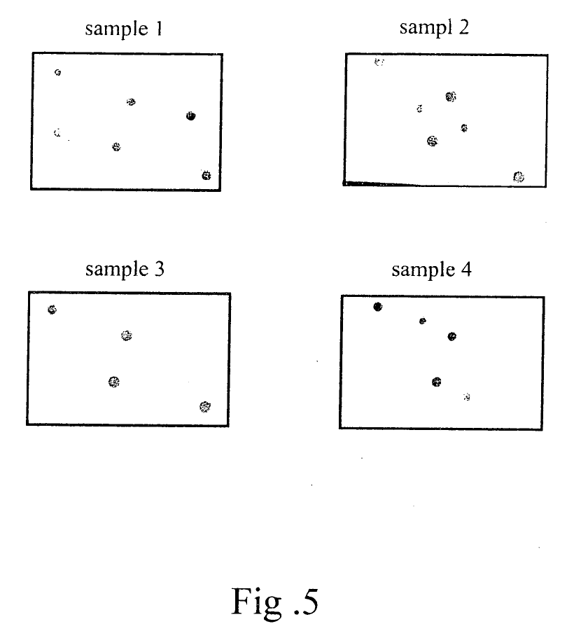

In addition, in another embodiment of the invention, the biological sample obtained from the patient is used and detected. The biochip 20 and the detection method described in the above are used for detecting and identifying the HPV subtypes contained in the sample according to the foresaid method. The results are shown in FIG. 5. When comparing the results shown in FIG. 5 and FIG. 3(b) based on the “SC” dot, the results show that HPV 53 is contained in the sample (1), HPV 45 is contained in the sample (2), HPV 52 is contained in the sample (3), and HPV 39 is contained in the sample (4). Therefore, when detecting the biological sample obtained from a patient, it is very clear that the biochip 20 can precisely identify the subtype of human papilloma viruses.

### EXAMPLE VI

According to another embodiment of the present invention, the carrier 11 could be a glass plate. When the carrier 11 is a glass plate, the detector 10 provided by the present invention is used for identifying the subtypes of human papilloma viruses according to the following hybridization steps.

4.1 The PCR product having CyS labeled thereon is purified by PCR Clean Up-M System (Viogene, USA), and the PCR product is precipitated in ethanol. Then, the PCR product is dried.

4.2 The precipitated DNA is dissolved in 12 μl of the buffer (2×SSC/0.1% SDS), and centrifugated for 1 minute, and then placed on boiled water for 2 minutes. Then, the mixture is placed on ice for 5 minutes.

4.3 The mixture is centrifugated for 30 seconds, and 10 μl of the mixture is added to the left side of the dot array 22. A cover slice is carefully covered on the dot array from the left side of the dot array to prevent the bubble formation. Then, the detector 10 is place in Humid Chamber (Sigma, USA), and the dot array is faces downward at 35□ for 4 hours or overnight.

4.4 The detector 10 is vertically placed in the solution A (2×SSC/1% SDS), and the detector is slightly oscillated apart from the cover slice. Then, the detector 20 is washed in a shaker at 160 rpm for 12 minutes.

4.5 The detector 10 is washed in the solution B (0.2×SSC/0.1% SDS) and oscillated at 35□ for 12 minutes. The detector 10 is washed in water. Then the detector 10 is dried.

4.6 The dried detector 10 is scanned by GenePix™4000 (Axon, USA), excited by the light having 635 nm of wavelength, and analyzed by GenePixPro 3.0 (Axon, USA).

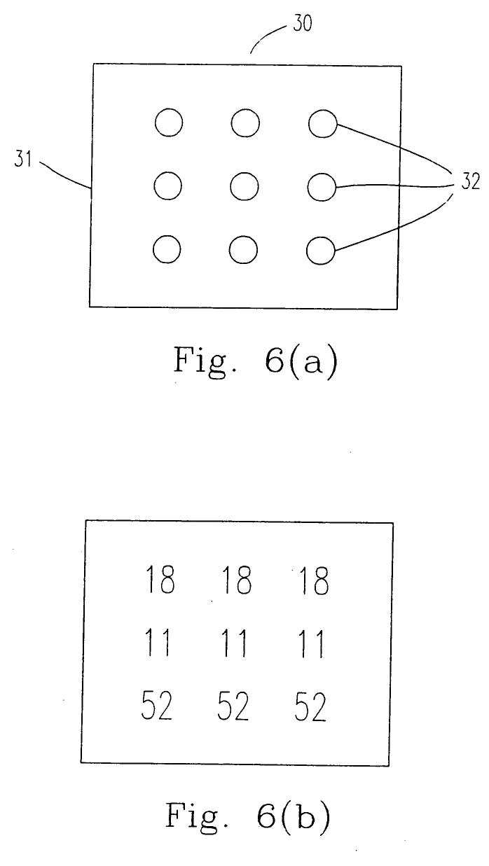

According to the above description, a biochip for specifically identifying the subtypes of human papilloma viruses contained in a biological sample is provided. Please refer to FIGS. 6(a) and (b). The biochip 30 includes a carrier 31 and a plurality of micro-dots 32 immobilized on the carrier 31. The carrier 31 is a glass plate. The micro-dots 32 are immobilized on the glass plate 31 according to the foresaid method. Each micro-dot 32 contains at least one oligonucleotide (15˜30mer), and each micro-dot 32 is used for specifically identifying a specific HPV subtype. The sequence of the oligonucleotide is selected from the foresaid list. The subtype of human papilloma viruses identified by each dot of the micro-dots 32 is illustrated in FIG. 6(b).

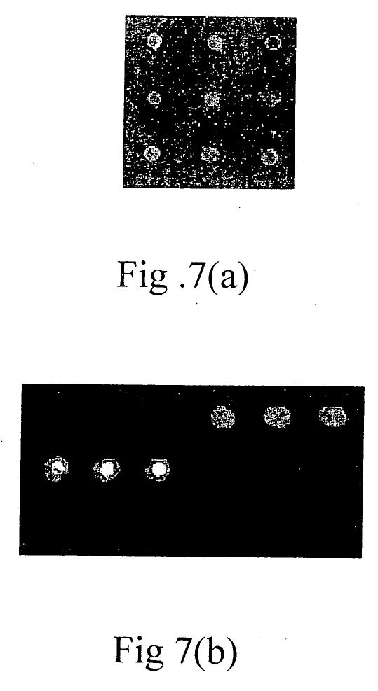

The biochip 30 is stained with SYBR Green II, scanned by GenePix™ 4000 (Axon, USA) and excited by the light having 635 nm of wavelength. The result is shown in FIG. 7(a). Preferably, the foresaid PCR amplified products are then detected by the biochip 30 according to the above steps and the results are shown in FIGS. 7(b). When comparing the results shown in FIG. 7(a) and FIG. 6(b), it is very clear that the biochip 30 can precisely identify the subtype of human papilloma viruses. The result clearly shows the exact positive micro-dots without any other false positive micro-dot. Besides, there is no cross reaction occurred in the detection, which proves that the biochip provided in the present invention has a very high specificity. Therefore, the biochip having different carriers (made of nylon membrane or glass plate) can obtain the same results and same specificities.

According to the above, the drawbacks in the conventional HPV detecting kit do not exist in the HPV detecting kit provided in the present invention. The HPV detecting kit of the present invention is able to diagnose multiple HPV subtypes (up to 39 different subtypes) at the same time, allowing the rapid and reliable detection and identification of HPV possibly present in a biological sample. Besides, an internal control is included in the detector to show whether the detecting process is well handled so that the detecting result is dependable. In addition, HPV detecting kit of the present invention has a high specificity and accuracy. Hence, the present invention not only has a novelty and a progressive nature, but also has an industry utility.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

1. (canceled)

2. (canceled)

3. (canceled)

4. (canceled)

5. (canceled)

6. (canceled)

7. (canceled)

8. A method for detecting and simultaneously diagnosing at least one subtype of human papilloma viruses (HPV) contained in a biological sample, by the use of a detector wherein said detector comprises:
a carrier,
a plurality of micro-dots immobilized on said carrier, wherein each micro-dot is for identifying one particular HPV subtype, and said HPV subtype is one selected from a group consisting of (HPV 6, HPV 11, HPV 16, HPV 18, HPV 26, HPV 31, HPV 32, HPV 33, HPV 35, HPV 37, HPV 39, HPV 42, HPV 43, HPV 44, HPV 45, HPV 51, HPV 52, HPV 53, HPV 54, HPV 55, HPV 56, HPV 58, HPV 59, HPV 61, HPV 62, HPV 66, HPV 67, HPV 68, HPV 69, HPV 70, HPV 72, HPV 74, HPV 82, HPV CP8061, HPV CP8034, HPV L1AES, HPV MM4, HPV MM7 and HPV MM8); and
at least one oligonucleotide sequence contained in each said micro-dot that is specific to said one particular HPV subtype.
wherein said at least one oligonucleotide sequence serves as a detection probe that hybridizes specifically with an L1 gene sequence of said one particular HPV subtype to form a hybridization complex as a detection indicator, so that each micro-dot identifies one particular HPV subtype via a corresponding oligonucleotide of said one particular HPV subtype, and thereby detecting and simultaneously identifying subtypes of human papilloma viruses,
wherein the method comprises the steps of:
amplifying an L1 gene fragment of human papilloma viruses (HPV) contained in said biological sample and obtaining an amplification product by polymerase chain reaction (PCR) using primers labeled with signaling substance;
hybridizing said amplification product with a said detector to form a hybridization complex;
removing said amplification product that is not hybridized; and
detecting said hybridization complex through detecting said signaling substance, thereby detecting and simultaneously identifying HPV subtypes contained in said biological sample.

9. The method according to claim 8, wherein said amplification product has a length of 450 base pairs by using MY09 as sense primer and MY11 as anti-sense primer in polymerase chain reaction (PCR).

10. The method according to claim 8, wherein said amplification product has a length of 190 base pairs by using MY11 as sense primer and GP6+ as anti-sense primer in polymerase chain reaction (PCR).

11. The method according to claim 8, wherein said signaling substance is biotin.

12. The method according to claim 11, wherein said biotin reacts with avidin-alkalinephosphatase to show said hybridization complex by presenting a particular color.

13. The method according to claim 8, wherein said signaling substance is a fluorescent substance.

14. The method according to claim 13, wherein said fluorescent substance is Cyanine 5.

15. (canceled)
